(19) United States
(12) Patent Application Publication
(10) Pub. No.: US 2012/0094855 A1
(43) Pub. Date: Apr. 19, 2012

(54) MEANS AND METHODS FOR DIAGNOSING PREDISPOSITION FOR TREATMENT EMERGENT SUICIDAL IDEATION (TESI)

(75) Inventors: Andreas Menke, Munich (DE); Elisabeth B. Binder, Munich (DE); Florian Holsboer, Munich (DE)

(73) Assignee: MAX PLANCK SOCIETY, Munich (DE)

(21) Appl. No.: 13/119,677

(22) PCT Filed: Sep. 7, 2009
(86) PCT No.: PCT/EP2009/061575

§ 371 Date: Sep. 30, 2011

(30) Foreign Application Priority Data
Sep. 18, 2008 (EP) 08016477.5
Mar. 12, 2009 (EP) 09003639.3

(51) Int. Cl. C40B 30/04; G01N 33/559; C12Q 1/68

(52) U.S. Cl. 506/9; 435/6.11; 204/456

(57) ABSTRACT

This invention relates to a method of diagnosing a predisposition for or the occurrence of treatment emergent suicidal ideation in an individual, the method comprising determining the presence or absence of one, more or all SNPs selected from the SNPs defined by SEQ ID NOs: 8, 18, 19, 47, 50, 52, 56, 64, 73, 86, 87, 92, 94, 95, 97 and 99.

This invention relates to method of diagnosing a predisposition for or the occurrence of treatment emergent suicidal ideation in an individual, the method comprising determining the presence or absence of one, more or all SNPs selected from the SNPs defined by SEQ ID NOs: 8, 18, 19, 47, 50, 52, 56, 64, 73, 86, 87, 92, 94, 95, 97 and 99.

In this specification, a number of documents including patent applications and manufacturer's manuals is cited. The disclosure of these documents, while not considered relevant for the patentability of this invention, is herewith incorporated by reference in its entirety. More specifically, all referenced documents are incorporated by reference to the extent as if each individual document was specifically and individually indicated to be incorporated by reference.

Major depression is a common psychiatric disease carrying a substantial loss of productivity and life quality and is associated with a significant morbidity and mortality with a suicide rate of about 15%^{1,2}. While antidepressants are the most effective treatment for depressive patients, there has been controversy if antidepressants, in particular serotonin reuptake inhibitors (SSRI) are implicated in the emergence or worsening of suicidal ideation³⁻⁵. Although treatment with antidepressants is associated with a significant reduction in suicides⁶⁻⁹ and was proven to have a suicide preventive effect¹⁰, there is some evidence that a subgroup of patients (about 5 to 15%) develop treatment emergent suicidal ideation (TESI) in the first weeks following treatment initiation and dose adjustments^{6, 11, 12}. In 2003, the Regulatory Agency of the British Department of Health warned physicians to avoid treatment with SSRI for depression in children and adolescents after studies showed an increase of agitation, hostility and suicidal behavior in this group^{13, 14}. This led to a public health advisory by the U.S. Food and Drug Administration (FDA) about the risk of suicidality in pediatric patients taking SSRIs for depression¹⁵. In 2005 the agency issued a black box warning and medication guide for a series of antidepressants (not only SSRIs) indicating that pediatric and adult patients may be at risk for this side effect. Recent studies showed a significant decrease in diagnosis and psychopharmacologic treatment of depressive episodes in children, adolescents but also adults following these warnings^{16, 17}. This was paralleled by an increase of suicide rates in the USA and the Netherlands between 2003 and 2005¹⁸ for the first time in a decade. Identification of a subgroup of patients at risk for treatment emergent suicidal behavior could therefore be critical to stop depriving patients of beneficial treatment options and to provide the risk group with closer monitoring.

While large family and twin studies estimate the heritability of suicidal behavior to be in the range of 30 to 55%^{19, 20}, no such formal evidence exists for TESI.

Markers for predicting the risk of developing TESI have previously been described; see for example US 2008/0102467. However, the markers described in this patent application deliver a significant number of false positives and false negatives when using them for predictive purposes and have not yet been replicated in an independent sample.

The technical problem underlying the present invention is the provision of alternative or improved means and methods for determining a predisposition for treatment emergent suicidal ideation in a patient.

Accordingly, this invention relates to a method of diagnosing a predisposition for or the occurrence of treatment emergent suicidal ideation in an individual, the method comprising determining the presence or absence of one, more or all SNPs selected from the SNPs defined by SEQ ID NOs: 8, 18, 19, 47, 50, 52, 56, 64, 73, 86, 87, 92, 94, 95, 97 and 99.

As discussed in more detail below, these 16 SNPs are the result from a double-filter procedure which has not been described in the art relating to the field of markers for treatment emergent suicidal ideation. More specifically, the 16 SNPs according to the main embodiment share the feature of being validated on a patient data set different from the training or discovery data set. To the inventors' best knowledge, such a set of validated SNPs for predicting or diagnosing TESI is not available in the art. The set of 16 SNPs is listed in Table 2 below.

The term “treatment emergent suicidal ideation (TESI)” is known in the art and designates the occurrence or enhanced occurrence of suicidal ideation in response to treatment, more specifically in response to treatment with anti-depressive drugs. To explain further, suicidal ideation may be a symptom of depressive disorders prior to beginning any therapy. A subgroup of patients (about 5 to 15%), however, develop and adverse response to treatment, the treatment being directed to an alleviation of the symptoms of depressive disorders, in that they develop or develop in enhanced form suicidal ideation upon the administration of anti-depressant drugs. While sufficient psychopharmacological treatment may eventually cure or at least alleviate this side effect, it is necessary to closely monitor those patients which have a predisposition to develop TESI. Moreover, the additional administration of anxiolytica and/or sedative medication is to be considered in such patients.

The term “predisposition” in relation to a disease has its established meaning in the art and is used accordingly here. To explain further, the term “predisposition” relates to the susceptibility to a disease that can be triggered under certain conditions. A person exhibiting a predisposition to a certain disease is not necessarily destined to develop the disease, but exhibits a risk to do so, wherein the risk exceeds the population average. As regards predisposition for TESI, the conditions which may serve as a trigger comprise, as explained above, the administration of anti-depressant drugs.

In addition to providing a method of diagnosing a predisposition for TESI, the main embodiment, in an alternative, provides a method of diagnosing the occurrence of TESI. While the former embodiment relates to patients which have not or have not yet developed TESI, the latter embodiment relates to patients which already suffer from TESI. The method of diagnosing the occurrence of TESI may be applied to the latter group of patients either as the only method of diagnosing TESI, or in conjunction with established methods of diagnosing TESI. When applying the method of diagnosing TESI according to the invention in conjunction with known methods of diagnosing TESI, the performance of the overall diagnostic method is improved. In particular, the number of falls positives and falls negatives is reduced.

The term “SNP” is well-known in the art and is shorthand for “single nucleotide polymorphism”. A SNP is a DNA sequence variation which is confined to a single position. Usually, polymorphisms are distinguished from mutations based on their prevalence. Sometimes a threshold of 1% prevalence in a population of individuals is considered for separating polymorphisms (more frequent) from mutations (less frequent). When present in an exon, and dependent on the occurrence of alternative splicing, the SNP may also be present in the mature mRNA. In the latter case, and depending on the degeneracy of the genetic code, a SNP may also be visible at the protein level. SNPs may furthermore be divided into SNPs occurring within a known locus, in the proximity of a known locus, and SNPs that are 5′ further away than 2 kb from the most 5′ feature of a gene and 3′ further away than 500 bases from most 3′ feature of a gene. Where applicable, loci where a SNP is located in and/or loci in the proximity of a SNP are indicated in the tables provided further below. Generally speaking, a SNP may or may not be associated with a certain phenotype, disease, or predisposition for a disease. The present inventors provide a plurality of SNPs, the association of which with TESI is disclosed herein for the first time.

SNPs are being annotated, collected and maintained in various databases including the SNP database of the National Centre for Biotechnology Information of the National Institutes of Health (NCBI), US. The SNPs according to the invention are referred to below by using “rs” identifiers as used in the SNP database of the NCBI, also known as “dbSNP”. More specifically, the rs identifiers refer to the dbSNP build 125, NCBI build 36.1/HG18. The SNP database of the NCBI is described in, for example, Sherry et al. (2001)²¹.

The SNPs according to the present invention are defined by the sequences comprised in the sequence listing. Generally speaking, a polymorphism may occur in at least two allelic forms, at least one of which may be referred to as the risk allele in those cases where an allelic form is associated with a disease or a risk to develop a disease. The sequences enclosed herewith and defining the SNPs of SEQ ID NOs: 1 to 100 throughout present the risk allele. In other words, presence of the respective sequence in a sample obtained from a patient is indicative of a risk to develop treatment emergent suicidal ideation. The respective allele which is not associated with a risk to develop TESI is indicated in the feature part of the respective entry of the sequence listing as “variation”. The relevant polymorphic position defining the SNP is the position indicated in the corresponding entries of Table 7. This position is located in the middle of the sequence of the respective entry of the sequence listing: This is position is position number 201; see also Table 7. In those cases where the sequence listing lists more than one variable position for a given entry, it is understood that the polymorphic position at position number 201 is intended for the purpose of the present invention.

While the respective entire sequence as presented in the sequence listing may be used to define the SNP according to the invention, it is also envisaged to use a subsequence of the sequence of the respective SEQ ID NO., said subsequence being, for example, 15, 17, 19, 21, 23 or 25 nucleotides in length and the variable position being located, for example, in the middle of the respective subsequence. Also subsequences consisting of an even number of nucleotides may be considered such as subsequences consisting of 16, 18, 20, 22 and 24 nucleotides, wherein the variable position may be, for example, one of the two central positions of the respective subsequence. The nucleobase characterizing the risk allele is furthermore presented in Tables 2 to 4. We note that the SNP as such is confined to the variable position itself. The length of the flanking sequence used for defining the SNP does not have any limiting effect, neither on the definition of the SNP nor on any specific method for determining presence or absence of the SNP, such methods being further detailed below. Preferably, the length of the flanking sequences is chosen such that a unique position in the genome is defined. This is why the SNP databases give preference to long flanking sequences. On the other hand it would equally be possible to define a SNP by (i) specifying the bases which may occur at the variable position and (ii) providing an “absolute” position on the given chromosome, for a given release of the sequence of the respective chromosome or genome.

The set of 100 SNPs defined by SEQ ID NOs: 1 to 100 has been obtained by determining SNPs associated with TESI in a first group of patients (also referred to as “discovery sample” herein). All 100 SNPs are characterized by statistically significant correlation with TESI and capability to predict TESI within this first group of patients. In a second step, a second group of patients has been considered. This second group represents an independent sample and is also referred to as “replication sample” herein. Each SNP of the set of 16 SNPs according to the main embodiment is characterized in that it correlates with TESI also in this second group in a statistically significant manner and is furthermore capable of predicting TESI in this second group of individuals.

To explain further, a discriminant analysis classifying patients using these 16 SNPs significantly associated with TESI in both a discovery and a replication sample revealed a 92% probability to classify TESI vs. non-TESI cases correctly in the discovery sample and a 85% probability in the replication sample. Specificity was 100% in discovery sample and 95% in replication sample, sensitivity was 72% in discovery sample and 50% in replication sample; see also Table 2 below. In Table 2, the performance of individual SNPs is also indicated.

The terms “discovery sample” and “replication sample” are commonly used in the art. The discovery sample is used for determining associations between polymorphisms and a phenotype such as TESI. The replication sample is an independent sample, i.e., a non-overlapping group of patients. It is used for validating the associations determined using the discovery sample. Examples of a discovery sample and a replication sample are disclosed in the Example.

In a preferred embodiment, the selected SNPs consist of or comprise the SNP defined by SEQ ID NO: 8. The SNP defined by SEQ ID NO: 8 is the SNP with the lowest p-value for the discovery group among the group of 16 SNPs according to the main embodiment.

In a further preferred embodiment, exactly 2, 3, 4, 5, 10 or 15 SNPs are used. When constructing subsets of 2, 3, 4, 5, 10 or 15 SNPs, preference is given to those SNPs which, when used alone, deliver the best prediction. Generally speaking, subsets of SNPs, i.e. in the present case subsets of less than 16 SNPs, exhibiting optimal predictive power on a given data set may be determined with tools known in the art when provided with the set of 16 SNPs according to the invention. Suitable tools are described in the Example enclosed herewith.

In another preferred embodiment, in addition to one, more or all SNPs selected from the SNPs defined by SEQ ID NOs: 8, 18, 19, 47, 50, 52, 56, 64, 73, 86, 87, 92, 94, 95, 97 and 99, one, more or all SNPs selected from the SNPs defined by SEQ ID NOs: 1 to 7, 9 to 17, 20 to 46, 48, 49, 51, 53 to 55, 57 to 63, 65 to 72, 74 to 85, 88 to 91, 93, 96, 98 and 100 are used. According to this embodiment, one, more or all validated SNPs may be used in combination with one, more or all SNPs as defined by the remaining 84 SNPs as comprised in the set of 100 SNPs as shown in Table 1.

In another preferred embodiment, instead of or in addition to a SNP as defined in any one of the embodiments above, a corresponding tagging SNP is used, wherein said tagging SNP is provided in Table 4. As discussed in more detail below, tagging SNPs are those SNPs which highly or fully correlate with a SNP as defined by any one of SEQ ID NOs: 1 to 100. The invention can be practiced by using one or more tagging SNPs instead of (or in addition to) one or more SNPs as defined herein above.

The present invention also provides a method of diagnosing a predisposition for or the occurrence of treatment emergent suicidal ideation in an individual, the method comprising determining the presence or absence of one or more SNPs selected from the SNPs defined by SEQ ID NOs: 1 to 100 in a sample obtained from a patient, wherein presence of the respective SNP(s) is/are indicative of said predisposition for or said occurrence of treatment emergent suicidal ideation, wherein said one SNP is or said more SNPs comprise the SNP defined by SEQ ID NO: 8. In a preferred embodiment of this method, said SNPs consist of or comprise the SNPs defined by SEQ ID NOs: 8, 18, 19, 47, 50, 52, 56, 64, 73, 86, 87, 92, 94, 95, 97 and 99.

Table 1 below presents the 100 SNPs according to the invention in the same order as they are presented in the enclosed sequence listing. More specifically, the SNPs are ordered according to the p-value. The p-value in this case is the likelihood that the observed association with TESI occurs by chance. The first column of Table 1 provides the database identifier of the respective SNP in dbSNP build 125. The corresponding sequences as presented in the enclosed sequence listing may also be retrieved from database NCBI build 36.1/HG18. The fifth column of the table presents the nucleobase present at the variable position in the risk allele. In addition to the sequence listing, also Table 7 presents the sequences of the SNPs of SEQ ID NOs:1 to 100.

TABLE 1
Table 1: Results from association analysis

| SNP | CHR | MAP | Gene | Risk allele | p-value |
|---|---|---|---|---|---|
| rs1630535 | 15 | 58297467 | | A | 1.3 × −7 |
| rs4724701 | 7 | 5495408 | #FBXL18 | A | 2 × 10−6 |
| rs1265235 | 6 | 4445680 | | A | 1.06 × −5 |
| rs2414660 | 15 | 58262633 | | T | 1.37 × −5 |
| rs12589623 | 14 | 47934734 | | A | 0.00002 |
| rs583338 | 18 | 39126230 | | G | 0.00003 |
| rs279542 | 3 | 9910772 | #IL17RE::2 | G | 0.00003 |
| rs279553 | 3 | 9909604 | #IL17RE::2 | G | 0.00003 |
| rs2774089 | 13 | 111885503 | | G | 0.00004 |
| rs1421780 | 5 | 174335276 | | A | 0.00005 |
| rs4907674 | 13 | 111900673 | | G | 0.00005 |
| rs7676106 | 4 | 190343908 | | C | 0.00006 |
| rs9949324 | 18 | 74181100 | | A | 0.00007 |
| rs4518023 | 20 | 39454182 | | A | 0.00008 |
| rs7704939 | 5 | 78109953 | #ARSB | A | 0.00008 |
| rs4544214 | 15 | 30719195 | #ARHGAP11A::2 | T | 0.00009 |
| rs6072343 | 20 | 39401601 | #LPIN3 | A | 0.00009 |
| rs965118 | 7 | 150809355 | #RHEB | A | 0.0001 |
| rs1109089 | 7 | 150800951 | #RHEB | G | 0.00011 |
| rs2980872 | 8 | 126486101 | | C | 0.00011 |
| rs2025949 | 13 | 92742277 | #GPC6 | G | 0.00012 |
| rs12142266 | 1 | 87070436 | | T | 0.00012 |
| rs9877859 | 3 | 112923378 | #PLCXD2 | C | 0.00012 |
| rs2070439 | 21 | 34933082 | | T | 0.00012 |
| rs12617566 | 2 | 167435454 | | A | 0.00013 |
| rs1958421 | 14 | 83249995 | | G | 0.00013 |
| rs9589698 | 13 | 92722965 | #GPC6 | C | 0.00013 |
| rs7645289 | 3 | 18047811 | | G | 0.00014 |
| rs2664537 | 20 | 39247142 | #ZHX3#PLCG1 | A | 0.00015 |
| rs2228246 | 20 | 39225477 | #PLCG1 | G | 0.00015 |
| rs2088138 | 17 | 44359721 | #SNF8#UBE2Z | T | 0.00015 |
| rs2839178 | 21 | 46503201 | #MCM3AP | A | 0.00016 |
| rs6500497 | 16 | 87338087 | #FLJ40448 | T | 0.00016 |
| rs11258903 | 10 | 14236785 | #FRMD4A | T | 0.00019 |
| rs2244057 | 14 | 61162625 | | C | 0.00019 |
| rs1459841 | 5 | 95936220 | | G | 0.0002 |
| rs7901463 | 10 | 59842383 | | C | 0.0002 |
| rs584762 | 11 | 31827333 | | C | 0.0002 |
| rs761453 | 11 | 31834575 | | A | 0.0002 |
| rs6035712 | 20 | 20773775 | | T | 0.0002 |
| rs554710 | 9 | 135171668 | | T | 0.00021 |
| rs6868846 | 5 | 59164510 | | A | 0.00021 |
| rs1013120 | 17 | 13485943 | | C | 0.00022 |
| rs649867 | 18 | 75501034 | | G | 0.00023 |
| rs6072317 | 20 | 39314930 | #ZHX3 | T | 0.00023 |
| rs4795069 | 17 | 30633359 | | A | 0.00026 |
| rs6948196 | 7 | 150844144 | #RHEB | T | 0.00029 |
| rs13403584 | 2 | 179550306 | | T | 0.00029 |
| rs7685314 | 4 | 190363247 | | A | 0.0003 |
| rs2662090 | 3 | 9066945 | #SRGAP3 | C | 0.0003 |
| rs6500498 | 16 | 87338145 | #FLJ40448 | G | 0.0003 |
| rs2074997 | 7 | 150805218 | #RHEB | G | 0.0003 |
| rs1204798 | 6 | 116650539 | #NT5DC1 | A | 0.00031 |
| rs12143647 | 1 | 228874868 | #COG2 | T | 0.00032 |
| rs3801033 | 7 | 6700627 | #ZNF12 | T | 0.00032 |
| rs10997044 | 10 | 67727448 | #CTNNA3 | A | 0.00035 |
| rs7350731 | 14 | 25179946 | | A | 0.00038 |
| rs9323737 | 14 | 83252414 | | G | 0.00038 |
| rs4881394 | 10 | 5103710 | | C | 0.0004 |
| rs4799159 | 18 | 74381450 | | A | 0.0004 |
| rs2270101 | 7 | 22902528 | | C | 0.00041 |
| rs356999 | 2 | 60665087 | | T | 0.00041 |
| rs618670 | 18 | 74187714 | | A | 0.00042 |
| rs7788668 | 7 | 22947162 | #DRCTNNB1A | A | 0.00045 |
| rs9332172 | 10 | 96721777 | #CYP2C9 | G | 0.00046 |
| rs902923 | 9 | 93373419 | | G | 0.00046 |
| rs4810622 | 20 | 45310632 | #PRKCBP1 | T | 0.00047 |
| rs6873640 | 5 | 123937398 | | C | 0.00047 |
| rs9302822 | 16 | 6482925 | #A2BP1 | A | 0.00047 |
| rs3753151 | 7 | 150815917 | #RHEB | C | 0.00051 |
| rs10490832 | 3 | 74427045 | #CNTN3 | G | 0.00052 |
| rs1545384 | 3 | 74434950 | #CNTN3 | A | 0.00052 |
| rs1884641 | 20 | 5987908 | #C20orf75 | G | 0.00052 |
| rs1958438 | 14 | 83230211 | | T | 0.00053 |
| rs4917639 | 10 | 96715524 | #CYP2C9 | C | 0.00054 |
| rs2279103 | 18 | 75574114 | #CTDP1 | T | 0.00057 |
| rs7307064 | 12 | 21525660 | #RECQL::2 | C | 0.00059 |
| rs6793017 | 3 | 2554126 | #CNTN4 | A | 0.00066 |
| rs523386 | 18 | 75628182 | | C | 0.00072 |
| rs1984151 | 6 | 94585819 | | T | 0.00076 |
| rs7719325 | 5 | 123500474 | | G | 0.00076 |
| rs1454333 | 4 | 92062152 | | A | 0.00078 |
| rs7146332 | 14 | 40847650 | | A | 0.00079 |
| rs11250017 | 8 | 10357428 | | C | 0.00079 |
| rs17175096 | 18 | 60871165 | | C | 0.00084 |
| rs2299965 | 7 | 150823594 | #RHEB | C | 0.00085 |
| rs2299967 | 7 | 150836143 | #RHEB | T | 0.00085 |
| rs720733 | 4 | 138216796 | | A | 0.00085 |
| rs635546 | 18 | 5927731 | | C | 0.00086 |
| rs7550277 | 1 | 239520957 | #RGS7 | C | 0.00086 |
| rs301191 | 3 | 177621532 | | A | 0.00089 |
| rs301193 | 3 | 177622218 | | C | 0.00089 |
| rs12583395 | 13 | 34117777 | | G | 0.00095 |
| rs1037448 | 11 | 60891107 | #CYBASC3 | T | 0.00096 |
| rs4939517 | 11 | 60883433 | #CYBASC3 | C | 0.00096 |
| rs4388301 | 6 | 138324067 | | C | 0.00096 |
| rs8095186 | 18 | 38167177 | | G | 0.00098 |
| rs1891877 | 10 | 23018034 | #PIP5K2A | C | 0.00098 |
| rs2348427 | 4 | 111633847 | #ENPEP | T | 0.00098 |
| rs9480684 | 6 | 107118917 | #RTN4IP1 | A | 0.00099 |

Best empiric associations calculated with Fisher Product Method over both allelic and genotypic tests with p < 0.001 and being significantly associated in both non-TESI comparison groups. The non-TESI comparison groups were 1) all individuals without increase in suicidality independent of baseline suicidality (no increase of the HAM-D item 3 over time; n = 329) and 2) a sub-group of 1), patients rating zero on item 3 throughout treatment (item 3 of the HAM-D = zero at allvisits; n = 79; see also example 1, Psychopathology and phenotype definition).
SNP: reference sequence identification number according to dbSNP;
CHR: chromosome number;
MAP: physical location of SNP on genome build HG18.

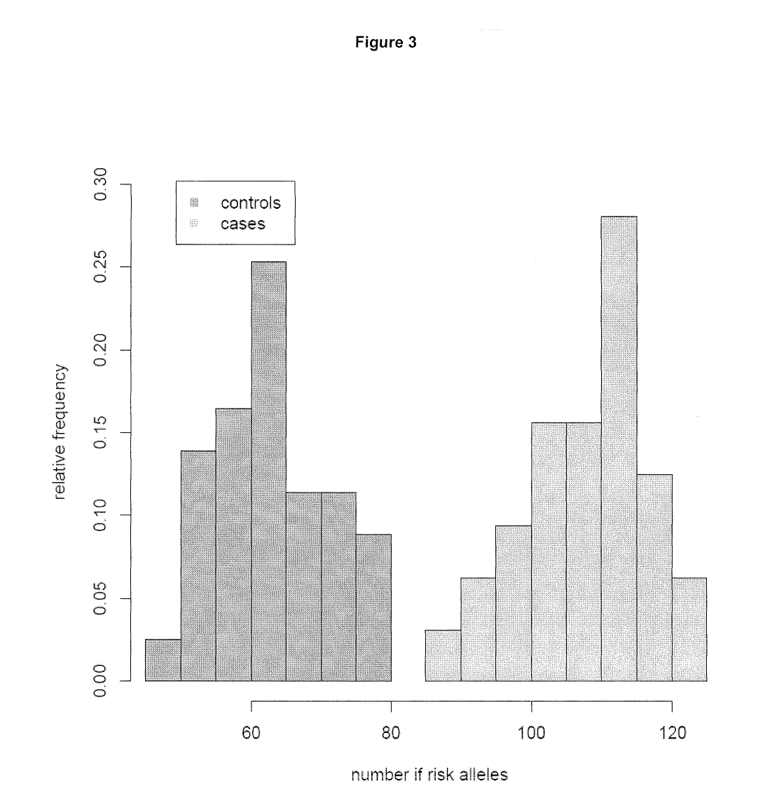

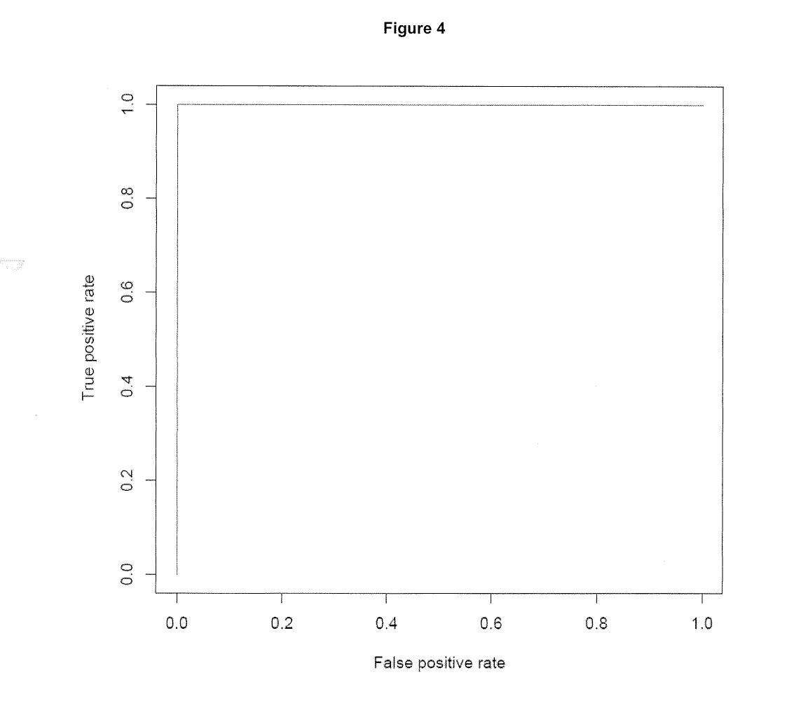

The SNPs according to the invention are characterized by a particularly high predictive value. As shown in the Example as well as in FIGS. 3 and 4 enclosed herewith, when using all 100 SNPs according to the invention, a perfect discrimination between risk patients and patients which do not exhibit a risk of developing TESI is achieved in the discovery sample.

The SNPs according to the invention are furthermore indicative of possible pathophysiological pathways relevant for TESI. Accordingly, they are indicative of candidate targets for therapeutic intervention. Genes encoding said candidate targets are indicated in Tables 1 to 4. It is of note that none of the genes harbouring the 16 SNPs have been associated with suicidality yet, however, regions around the genes RHEB, TMEM138 and CYBASC3 have been implicated with bipolar disorder in linkage studies. Of note is that 6 of 11 RHEB SNPs are associated with TESI, even if they are in high linkage disequilibrium. Most of the genes are involved in cell growth or organization (RHEB, ENPEP, TNNA3 and JAGN1); SRGAP3 is associated with neuronal signalling.

In a preferred embodiment, said SNPs consist of or comprise the SNPs defined by SEQ ID NOs: 8, 18, 19, 47, 50, 52, 56, 64, 73, 86, 87, 92, 94, 95, 97 and 99. This is the set of 16 validated SNPs as described above.

TABLE 2
Table 2: Best empiric associations calculated with allelic tests and being significantly
associated in both discovery and replication sample.

| | | | Gene | | | Discovery Sample | |
|---|---|---|---|---|---|---|---|
| | | | (Closest gene | | Risk | P value | P value |
| SNP | CHR | MAP | and distance) | Location | allele | FM Gr B | Group A |
| rs1037448 | 11 | 60891107 | TMEM138 | INTRONIC | T | 0.00096 | 1.48 × −5 |
| rs10997044 | 10 | 67727448 | CTNNA3 | INTRONIC | A | 0.00035 | 0.001 |
| rs1109089* | 7 | 150800951 | RHEB | INTRONIC | G | 0.00011 | 1.67 × −5 |
| rs1884641 | 20 | 5987908 | (C20orf755233) | INTERGENIC | G | 0.00052 | 0.01045 |
| rs2074997* | 7 | 150805218 | RHEB | INTRONIC | G | 0.0003 | 3.49 × −5 |
| rs2299965* | 7 | 150823594 | RHEB | INTRONIC | C | 0.00085 | 0.00013 |
| rs2299967* | 7 | 150836143 | RHEB | INTRONIC | T | 0.00085 | 0.00013 |
| rs2348427 | 4 | 111633847 | ENPEP | INTRONIC | T | 0.00098 | 0.00659 |
| rs2662090 | 3 | 9066945 | SRGAP3 | INTRONIC | C | 0.0003 | 0.00051 |
| rs279553 | 3 | 9909604 | JAGN1 | SYNONYMOUS_CODING | G | 0.00003 | 0.00077 |
| rs301193 | 3 | 177622218 | (N/A) | INTERGENIC | C | 0.00089 | 4.45 × −5 |
| rs4939517 | 11 | 60883433 | CYBASC3 | INTRONIC | C | 0.00096 | 1.96 × −5 |
| rs6948196* | 7 | 150844144 | RHEB | INTRONIC | T | 0.00029 | 3.13 × −5 |
| rs7788668 | 7 | 22947162 | (FAM126A- | INTERGENIC | A | 0.00045 | 0.00294 |
| | | | 2622) | | | | |
| rs8095186 | 18 | 38167177 | (PIK3C3 | INTERGENIC | G | 0.00098 | 0.00057 |
| | | | 251736) | | | | |
| rs965118* | 7 | 150809355 | RHEB | INTRONIC | A | 0.0001 | 7.1 × −5 |

| | Discovery Sample | | Replication Sample | | | |
|---|---|---|---|---|---|---|
| | P value | OR | P value | P value | P value | OR |
| SNP | Group B | Gr B | FM Gr B | Group A | Group B | Gr B |
| rs1037448 | 0.00093 | 3.3 | 0.035 | 0.012 | 0.035 | 1.8 |
| | | (1.6-6.8) | | | | (1.0-3.4) |
| rs10997044 | 0.00101 | 3.1 | 0.141 | 0.046 | 0.113 | 1.7 |
| | | (1.6-6.1) | | | | (1.0-3.3) |
| rs1109089* | 0.00011 | 3.5 | 0.041 | 0.004 | 0.03 | 1.7 |
| | | (1.8-6.6) | | | | (1.0-2.9) |
| rs1884641 | 0.00618 | 2.5 | 0.018 | 0.009 | 0.014 | 1.8 |
| | | (1.3-4.8) | | | | (1.0-3.0) |
| rs2074997* | 0.00024 | 3.2 | 0.041 | 0.003 | 0.032 | 1.6 |
| | | (1.7-6.0) | | | | (1.0-2.7) |
| rs2299965* | 0.00073 | 3.0 | 0.024 | 0.005 | 0.018 | 2.0 |
| | | (1.6-5.5) | | | | (1.1-3.5) |
| rs2299967* | 0.00073 | 3.0 | 0.019 | 0.003 | 0.018 | 2.2 |
| | | (1.6-5.5) | | | | (1.2-4.0) |
| rs2348427 | 0.00177 | 2.5 | 0.043 | 0.054 | 0.027 | 2.0 |
| | | (1.4-4.6) | | | | (1.2-3.3) |
| rs2662090 | 0.00026 | 3.2 | 0.041 | 0.105 | 0.037 | 1.8 |
| | | (1.7-6.1) | | | | (1.1-3.0) |
| rs279553 | 8 × −5 | 4.1 | 0.039 | 0.068 | 0.032 | 1.9 |
| | | (1.8-9.1) | | | | (1.0-3.4) |
| rs301193 | 0.00127 | 3.1 | 0.005 | 0.004 | 0.004 | 2.3 |
| | | (1.6-6.1) | | | | (1.3-4.3) |
| rs4939517 | 0.00093 | 3.3 | 0.079 | 0.01 | 0.056 | 1.7 |
| | | (1.6-6.8) | | | | (1.0-3.3) |
| rs6948196* | 0.00023 | 3.2 | 0.027 | 0.004 | 0.022 | 2.1 |
| | | (1.7-6.0) | | | | (1.1-3.7) |
| rs7788668 | 0.00096 | 2.8 | 0.052 | 0.04 | 0.042 | 1.7 |
| | | (1.5-5.1) | | | | (1.0-2.8) |
| rs8095186 | 0.00148 | 4.1 | 0.04 | 0.031 | 0.034 | 2.0 |
| | | (1.8-9.2) | | | | (1.0-3.9) |
| rs965118* | 8 × −5 | 3.6 | 0.055 | 0.005 | 0.044 | 1.7 |
| | | (1.9-6.7) | | | | (1.0-2.9) |

TESI positive: discovery n = 32; replication n = 42.
The non-TESI comparison groups were A) all individuals without increase in suicidality independent of baseline suicidality (no increase of the HAM-D item 3 over time; discovery n = 329; replication n = 434) and B) a sub-group of A), patients rating zero on item 3 throughout treatment (item 3 of the HAM-D = zero at all visits; discovery n = 79; replication n = 149; see also example 1, subsection entitled “Psychopathology and phenotype definition”).
SNP: reference sequence identification number according to dbSNP;
CHR: chromosome number;
MAP: physical location of SNP on genome build HG18. Allelic p-values are shown.
FM = Fisher Product Method over both allelic and genotypic tests.
*SNPs with LD > 0.8. OR = odds ratios based on comparison between cases and control group B.

The present invention furthermore provides a method of diagnosing a predisposition for or the occurrence of treatment emergent suicidal ideation in an individual, the method comprising determining the presence or absence of one or more SNPs selected from the SNPs defined by SEQ ID NOs: 1 to 100 in a sample obtained from a patient, wherein presence of the respective SNP(s) is/are indicative of said predisposition for or said occurrence of treatment emergent suicidal ideation.

In a preferred embodiment of the methods of the invention, exactly 2, 3, 4, 5, 10, 15, 16 or 18 SNPs are used.

In a further preferred embodiment, said one SNP is or said more SNPs comprise the SNP defined by SEQ ID NO: 1. This is the SNP being mentioned first in Table 1. When selecting subsets of SNPs, preference is given to subsets comprising SNPs with low p-values. P-values are indicated in Table 1.

In a further preferred embodiment, said SNPs consist of or comprise the SNPs defined by (a) SEQ ID NOs: 1, 3, 66 and 78; or (b) SEQ ID NOs: 1, 3, 6, 9, 21, 23, 29, 32, 38, 53, 55, 59, 61, 62, 66, 76, 78 and 94.

The set of four SNPs according to embodiment (a) has been shown to correctly predict a risk of developing TESI in 90% of the patients analyzed.

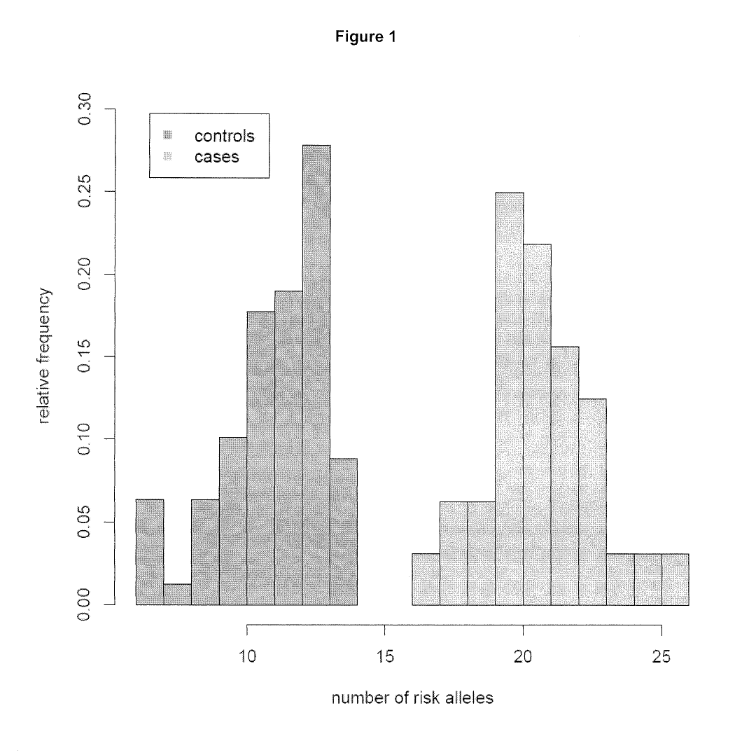

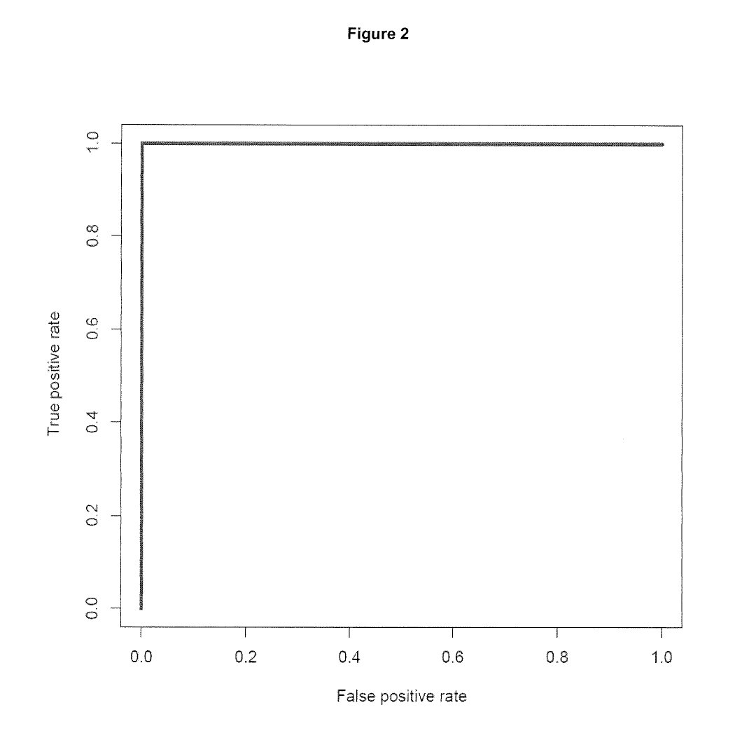

The set of 18 SNPs according to embodiment (b) provides correct predictions for 100% of the patients analyzed (see FIGS. 1 and 2 enclosed herewith). Table 3 provides a listing of the 18 SNPs characterized by SEQ ID NOs: 1, 3, 6, 9, 21, 23, 29, 32, 38, 53, 55, 59, 61, 62, 66, 76, 78 and 94. It is noteworthy that also this subset of 18 SNPs achieves the same discriminatory power as does the full set of 100 SNPs; see also FIGS. 1 and 2. When using the subset of 18 SNPs according to the invention or the respective subsequences thereof as defined herein above, individuals with 16 or more risk alleles are classified as TESI positive. When using 100 SNPs as defined by SEQ ID NOs. 1 to 100 or the respective subsequences thereof as defined herein above, individuals with 85 or more risk alleles are classified as TESI positive. Even single SNPs, in particular those with low P-values, allow excellent prediction.

TABLE 3
Table 3: Results from association analysis, subset with 18 SNPs

| | | | | Closest | Distance to | Risk | |
|---|---|---|---|---|---|---|---|
| SNP | CHR | MAP | Gene | gene | cl. gene | allele | P value |
| rs1630535 | 15 | 58297467 | | ANXA2P3 | −129157 | A | 1.3 × −7 |
| rs1265235 | 6 | 4445680 | | Q5G014 | −110164 | A | 1.06 × −5 |
| rs583338 | 18 | 39126230 | | SYT4 | 14801 | G | 0.00003 |
| rs2774089 | 13 | 111885503 | | C13orf28 | −193130 | G | 0.00004 |
| rs2025949 | 13 | 92742277 | GPC6 | | | G | 0.00012 |
| rs9877859 | 3 | 112923378 | PLCXD2 | | | C | 0.00012 |
| rs2664537 | 20 | 39247142 | ZHX3 | | | A | 0.00015 |
| rs2839178 | 21 | 46503201 | MCM3AP | | | A | 0.00016 |
| rs584762 | 11 | 31827333 | | PAX6 | 31249 | C | 0.0002 |
| rs1204798 | 6 | 116650539 | NT5DC1 | | | A | 0.00031 |
| rs3801033 | 7 | 6700627 | ZNF12 | | | T | 0.00032 |
| rs4881394 | 10 | 5103710 | | AKR1C3 | −22872 | C | 0.0004 |
| rs2270101 | 7 | 22902528 | | FAM126A | −47256 | C | 0.00041 |
| rs356999 | 2 | 60665087 | | BCL11A | 30951 | T | 0.00041 |
| rs902923 | 9 | 93373419 | ROR2 | | | G | 0.00046 |
| rs2279103 | 18 | 75574114 | CTDP1 | | | T | 0.00057 |
| rs6793017 | 3 | 2554126 | CNTN4 | | | A | 0.00066 |
| rs1037448 | 11 | 60891107 | TMEM138 | | | T | 0.00096 |

Best empiric associations calculated with Fisher Product Method over both allelic and genotypic tests with p < 0.001 and being significantly associated in both non-TESI comparison groups.
SNP: reference sequence identification number according to dbSNP;
CHR: chromosome number;
MAP: physical location of SNP on genome build HG18.

Subsets of SNPs, i.e. in the present case subsets of less than 100 SNPs, exhibiting optimal predictive power on a given data set may be determined with tools known in the art when provided with the set of 100 SNPs according to the invention. Suitable tools are described in the examples enclosed herewith.

As regards the means and methods for determining presence or absence of a given SNP, numerous suitable methods are known in the art. Any of these methods, either alone or in combination, may be used for determining presence or absence of one or more SNPs according to the invention in a sample. In a preferred embodiment, said determining is effected by allele specific hybridization, allele specific oligonucleotide ligation, primer extension, minisequencing, mass spectroscopy, heteroduplex analysis, single strand conformational polymorphism, denaturing gradient gel electrophoresis, microarray analysis, temperature gradient gel electrophoresis or combinations thereof.

To explain further, in allele specific hybridization a probe is used which is preferably strictly complementary to a region of the target nucleic acid comprising the SNP in question. Hybridization conditions are chosen which allow to distinguish between full complementarity and a single mismatch.

Allele specific oligonucleotide ligation is another method for detecting alleles that differ by a single base. A pair of oligonucleotide probes that hybridize to adjacent segments of the target nucleic acid are used. The oligomer on the 5′ side of the pair is an allele specific oligonucleotide in that it is strictly complementary to one specific allele of the target nucleic acid sequence, in the present case to the risk allele. The last base at the 3′ end of this oligonucleotide corresponds to the SNP. The oligomer on the 3′ side of the pair is the same for both different alleles. In case the risk allele is present, both oligonucleotides hybridize completely and are amenable to ligation with DNA ligase. In case the risk allele is not present, hybridization is not complete and no ligation is possible. Detecting the ligation product corresponds to detecting the risk allele. A variety of suitable detection schemes for the ligation product are known in the art.

In a primer extension assay according to the invention, nucleic acid comprised in the patient sample is hybridized to a primer complementary to the region adjacent to the SNP site. Dideoxyribonucleotides (ddNTPS) and DNA polymerase are added to the mixture and the primer is extended by a single nucleotide. The single nucleotide added is dependent on the allele of the amplified DNA. Primer extension biochemistry can be coupled with a variety of detection schemes, comprising fluorescence, fluorescence polarization (FP), luminescence and mass spectrometry (MS). Primer extension is sometimes also referred to as minisequencing.

Preferably, the hybridization of said primer to said nucleic acid is specific. Means of ensuring specificity of hybridization according to the present invention are known in the art and include stringent hybridization conditions. The term “stringent hybridization conditions”, as used in the description of the present invention, is well known to the skilled artisan. Appropriate stringent hybridization conditions for each sequence may be established by a person skilled in the art on well-known parameters such as temperature, composition of the nucleic acid molecules, salt conditions etc.; see, for example, Sambrook et al., “Molecular Cloning, A Laboratory Manual”; CSH Press, Cold Spring Harbor, 1989 or Higgins and Hames (eds.), “Nucleic acid hybridization, a practical approach”, IRL Press, Oxford 1985, see in particular the chapter “Hybridization Strategy” by Britten & Davidson, 3 to 15. Stringent hybridization conditions are, for example, conditions comprising overnight incubation at 42° C. in a solution comprising: 50% formamide, 5×SSC (750 mM NaCl, 75 mM trisodium citrate), 50 mM sodium phosphate (pH 7.6), 5×Denhardt's solution, 10% dextran sulfate, and 20 micrograms/ml denatured, sheared salmon sperm DNA, followed by washing the filters in 0.1×SSC at about 65°. Other stringent hybridization conditions are for example 0.2×SSC (0.03 M NaCI, 0.003M Natriumcitrat, pH 7) at 65° C.

Microarrays inter alia provide a miniaturized implementation of a plurality of allele specific hybridization assays in arrayed format. Microarrays for SNP detection are known in the art and available from a variety of manufacturers including Affymetrix. These microarrays include oligonucleotide microarrays.

In a further preferred embodiment, said treatment emergent suicidal ideation occurs or may occur in a patient being administered selective serotonin re-uptake inhibitors, selective noradrenalin re-uptake inhibitors, dual serotonin and noradrenalin re-uptake inhibitors and/or tricyclic antidepressants, and optionally being administered neuroleptics, mood stabilizers and/or benzodiazepines. As explained in the background section herein above, antidepressant drugs in general, and in particular selective serotonin reuptake inhibitors may be causative of TESI.

In a further preferred embodiment, said individual is a child or adolescent. As stated in the background section herein above, children and adolescents are considered to be particularly effected by drug induced TESI.

In a further preferred embodiment, said determining comprises isolating a nucleic acid from said sample.

Means and methods for isolating nucleic acids from a sample taken from an individual are well-known in the art²². Preferred samples according to the invention are samples comprising or consisting of body fluids. Generally speaking, body fluids are liquid components of living organisms. Preferred body fluids include blood, serum, saliva, semen, vaginal secretions as well amniotic, cerebrospinal, synovial, pleural, peritoneal and pericardial fluids. The term “nucleic acid” as used herein includes DNA such as cDNA or genomic DNA, and RNA. It is understood that the term “RNA” as used herein comprises all forms of RNA including mRNA, non-coding RNA, tRNA and rRNA. The term “non-coding RNA” in turn includes siRNA (small interfering RNA), miRNA (micro RNA), rasiRNA (repeat associated RNA), snoRNA (small nucleolar RNA) and snRNA (small nuclear RNA). A preferred RNA is mRNA.

The present invention also relates to a kit comprising (a) one or more agents suitable for determining presence or absence of two or more SNPs, said SNPs being defined by SEQ ID NOs: 1 to 100; and (b) optionally a manual with instructions for performing the method of the invention. Agents suitable for determining presence or absence of SNPs are well-known in the art^{23, 24}.

In a preferred embodiment of the kit according to the invention, said agents are selected from primers and probes for determining presence or absence of one or more SNPs of SEQ ID NOs: 1 to 100. Probes and primers may be used in methods such as allele specific hybridization, allele specific oligonucleotide ligation and primer extension as mentioned herein above.

In a further preferred embodiment, said more SNPs are at least 2, 3, 4, 5, 10, 15, 16, 18, 20, 30, 40, 50, 60, 70, 80, 90 or 100 SNPs.

Specific preferred sets of SNPs are those defined herein above. For example, a preferred set of SNPs is the set of SNPs defined by SEQ ID NOs: 8, 18, 19, 47, 50, 52, 56, 64, 73, 86, 87, 92, 94, 95, 97 and 99.

In preferred embodiments of the method or the kit of the invention, respectively, instead of or in addition to a SNP as defined in these claims, a corresponding tagging SNP is used, wherein said tagging SNP is provided in Table 4. Sequences of the tagging SNPs are presented in the sequence listing (SEQ ID NOs: 101 to 813).

TABLE 4
Table 4: Subset of 100 associated SNPs with tagged SNPs (r² > 0.8).

| | | Closest | distance to | |
|---|---|---|---|---|
| SNP | type | gene | gene | Tagged_SNP |
| rs1630535 | INTERGENIC | ANXA2P3 | −129157 | rs1872133 |
| | | | | rs1872132 |
| | | | | rs2054680 |
| | | | | rs336536 |
| | | | | rs163135 |
| | | | | rs12148406 |
| | | | | rs336528 |
| | | | | rs17270048 |
| | | | | rs17270055 |
| | | | | rs338446 |
| | | | | rs7182941 |
| | | | | rs7183074 |
| | | | | rs10851673 |
| | | | | rs12912083 |
| | | | | rs1680195 |
| | | | | rs16942291 |
| | | | | rs12148854 |
| | | | | rs8028748 |
| | | | | rs1680222 |
| | | | | rs1680221 |
| | | | | rs12899914 |
| rs4724701 | SYNONYMOUS_CODING | FBXL18 | 0 | rs7808325 |
| | | | | rs3801058 |
| | | | | rs7806101 |
| | | | | rs10216189 |
| | | | | rs4724699 |
| | | | | rs7799970 |
| | | | | rs4077245 |
| | | | | rs4560713 |
| rs1265235 | INTERGENIC | Q5G014_HUMAN | −110164 | rs1265237 |
| | | | | rs1265234 |
| | | | | rs7774098 |
| rs2414660 | INTERGENIC | ANXA2P3 | −163991 | rs338424 |
| | | | | rs2136559 |
| | | | | rs7165903 |
| | | | | rs7173200 |
| | | | | rs7173250 |
| | | | | rs900634 |
| | | | | rs7166014 |
| | | | | rs4775243 |
| | | | | rs2136557 |
| rs12589623 | INTERGENIC | N/A | −9 | rs1621025 |
| | | | | rs1769522 |
| | | | | rs11157633 |
| | | | | rs7158518 |
| rs279553 | SYNONYMOUS_CODING | JAGN1 | 0 | rs279558 |
| | | | | rs279542 |
| | | | | rs33970214 |
| rs279542 | 3PRIME_UTR | JAGN1 | 0 | rs279558 |
| | | | | rs279553 |
| | | | | rs33970214 |
| rs583338 | INTERGENIC | SYT4 | 14801 | rs816750 |
| | | | | rs310095 |
| | | | | rs310091 |
| | | | | rs310086 |
| | | | | rs310087 |
| | | | | rs310089 |
| | | | | rs618486 |
| rs2774089 | INTERGENIC | SOX1 | 111483 | rs2576497 |
| | | | | rs1946770 |
| | | | | rs100224 |
| | | | | rs188122 |
| rs1421780 | INTRONIC | Q6ZNE8_HUMAN | 0 | |
| rs4907674 | INTERGENIC | SOX1 | 126653 | |
| rs7676106 | INTERGENIC | LOC728856 | −48091 | rs9312392 |
| | | | | rs9312393 |
| | | | | rs7689338 |
| | | | | rs4241859 |
| | | | | rs4241860 |
| | | | | rs4241862 |
| | | | | rs4273547 |
| | | | | rs4282263 |
| | | | | rs6553287 |
| | | | | rs6820247 |
| | | | | rs10866319 |
| rs9949324 | INTERGENIC | N/A | −9 | rs9949519 |
| | | | | rs611509 |
| | | | | rs599612 |
| | | | | rs603942 |
| | | | | rs677233 |
| rs7704939 | 3PRIME_UTR | ARSB | 0 | |
| rs4518023 | INTERGENIC | CHD6 | −10401 | rs6065347 |
| | | | | rs4810317 |
| rs4544214 | UPSTREAM | ARHGAP11A | 46 | rs11632524 |
| | | | | rs8037818 |
| | | | | rs4643284 |
| rs6072343 | UPSTREAM | LPIN3 | −1372 | rs6065333 |
| | | | | rs16985655 |
| | | | | rs16985677 |
| rs965118 | INTRONIC | RHEB | 0 | rs17713386 |
| | | | | rs4726029 |
| | | | | rs875588 |
| | | | | rs1109089 |
| | | | | rs17713697 |
| | | | | rs2074998 |
| | | | | rs2074997 |
| | | | | rs17713890 |
| | | | | rs2299965 |
| | | | | rs2284264 |
| | | | | rs11772458 |
| | | | | rs2299967 |
| | | | | rs3789817 |
| | | | | rs758666 |
| | | | | rs736645 |
| | | | | rs4298422 |
| | | | | rs2374261 |
| | | | | rs12112989 |
| | | | | rs6980020 |
| | | | | rs12112134 |
| | | | | rs6948196 |
| | | | | rs6943752 |
| | | | | rs7794922 |
| rs1109089 | INTRONIC | RHEB | 0 | rs17713386 |
| | | | | rs4726029 |
| | | | | rs875588 |
| | | | | rs7805967 |
| | | | | rs17713697 |
| | | | | rs2074998 |
| | | | | rs2074997 |
| | | | | rs17713890 |
| | | | | rs2299961 |
| | | | | rs965118 |
| | | | | rs3753151 |
| | | | | rs2299965 |
| | | | | rs2284264 |
| | | | | rs11772458 |
| | | | | rs2299967 |
| | | | | rs3789817 |
| | | | | rs758666 |
| | | | | rs736645 |
| | | | | rs4298422 |
| | | | | rs2374261 |
| | | | | rs12112989 |
| | | | | rs6980020 |
| | | | | rs12112134 |
| | | | | rs6948196 |
| | | | | rs6943752 |
| | | | | rs7794922 |
| rs2980872 | INTERGENIC | TRIB1 | −25643 | rs2954006 |
| rs12142266 | INTERGENIC | SEP15_HUMAN | −30281 | |
| rs9877859 | INTRONIC | PHLDB2 | 0 | rs4682298 |
| rs2025949 | INTRONIC | GPC6 | 0 | rs7338831 |
| | | | | rs9561295 |
| | | | | rs9589698 |
| | | | | rs9523990 |
| | | | | rs9523991 |
| | | | | rs4142598 |
| | | | | rs9523998 |
| | | | | rs9561304 |
| | | | | rs956036 |
| | | | | rs1475449 |
| rs2070439 | INTERGENIC | RCAN1 | 23780 | |
| rs12617566 | INTERGENIC | XIRP2 | −32784 | |
| rs9589698 | INTRONIC | GPC6 | 0 | rs7338831 |
| | | | | rs9561295 |
| | | | | rs9523990 |
| | | | | rs9523991 |
| | | | | rs4142598 |
| | | | | rs9523998 |
| | | | | rs2025949 |
| | | | | rs9561304 |
| | | | | rs956036 |
| | | | | rs1475449 |
| rs1958421 | INTERGENIC | N/A | −9 | rs1958438 |
| | | | | rs10139321 |
| | | | | rs10132490 |
| | | | | rs10143458 |
| | | | | rs9671273 |
| | | | | rs1952534 |
| | | | | rs1958436 |
| | | | | rs11159605 |
| | | | | rs7359138 |
| | | | | rs1958435 |
| | | | | rs1958434 |
| | | | | rs1958433 |
| | | | | rs2149684 |
| | | | | rs2183203 |
| | | | | rs1958432 |
| | | | | rs2149683 |
| | | | | rs8010141 |
| | | | | rs1958430 |
| | | | | rs1958429 |
| | | | | rs1958428 |
| | | | | rs1958426 |
| | | | | rs1958425 |
| | | | | rs1958424 |
| | | | | rs1958423 |
| | | | | rs1958422 |
| | | | | rs10132081 |
| | | | | rs10132130 |
| | | | | rs10147226 |
| | | | | rs1958420 |
| | | | | rs10873350 |
| | | | | rs10140080 |
| | | | | rs9323737 |
| | | | | rs4545730 |
| | | | | rs1952530 |
| | | | | rs1952529 |
| | | | | rs12431659 |
| | | | | rs12434622 |
| | | | | rs10141403 |
| | | | | rs9323738 |
| | | | | rs9323739 |
| | | | | rs1952528 |
| | | | | rs1958418 |
| | | | | rs877800 |
| | | | | rs2372550 |
| | | | | rs1952545 |
| | | | | rs1952544 |
| | | | | rs10747302 |
| | | | | rs4899838 |
| | | | | rs11159610 |
| | | | | rs11159611 |
| | | | | rs7494095 |
| | | | | rs10132137 |
| | | | | rs4899839 |
| | | | | rs10135095 |
| | | | | rs2888357 |
| | | | | rs4143911 |
| | | | | rs1958458 |
| | | | | rs2372553 |
| | | | | rs10147836 |
| | | | | rs10139812 |
| | | | | rs1958456 |
| | | | | rs1958455 |
| | | | | rs2372523 |
| | | | | rs2372524 |
| | | | | rs10143348 |
| | | | | rs9323740 |
| | | | | rs10130591 |
| | | | | rs1958449 |
| rs7645289 | INTERGENIC | TBC1D5 | 290409 | rs4132218 |
| | | | | rs7627583 |
| | | | | rs9859212 |
| | | | | rs6790525 |
| | | | | rs4688898 |
| | | | | rs9861146 |
| | | | | rs2203242 |
| rs2088138 | 3PRIME_UTR | UBE2Z | 0 | |
| rs2228246 | N/A | N/A | −9 | |
| rs2664537 | 3PRIME_UTR | ZHX3 | 0 | rs2228246 |
| | | | | rs6072299 |
| | | | | rs6072302 |
| | | | | rs7261917 |
| | | | | rs6065325 |
| | | | | rs6072317 |
| | | | | rs6065326 |
| | | | | rs6072320 |
| | | | | rs6072321 |
| | | | | rs6072322 |
| rs6500497 | 3PRIME_UTR | Q8N7R2_HUMAN | 0 | rs7185630 |
| | | | | rs2242166 |
| | | | | rs2242164 |
| | | | | rs6500498 |
| rs2839178 | INTRONIC | MCM3AP | 0 | |
| rs11258903 | INTRONIC | FRMD4A | 0 | rs11258902 |
| | | | | rs10159816 |
| rs2244057 | INTERGENIC | HIF1A | −69366 | rs2253980 |
| | | | | rs2246928 |
| | | | | rs1006529 |
| | | | | rs698028 |
| rs1459841 | INTERGENIC | CAST | −87312 | |
| rs7901463 | INTERGENIC | TFAM | 12223 | rs10763541 |
| | | | | rs2081692 |
| | | | | rs7903523 |
| | | | | rs2101237 |
| | | | | rs2086784 |
| | | | | rs2393422 |
| | | | | rs1427219 |
| | | | | rs919166 |
| | | | | rs919167 |
| | | | | rs10826187 |
| | | | | rs10826188 |
| | | | | rs6481390 |
| | | | | rs7071862 |
| | | | | rs7896213 |
| | | | | rs7900673 |
| | | | | rs7916849 |
| | | | | rs10763545 |
| | | | | rs4948298 |
| | | | | rs4948299 |
| | | | | rs4948513 |
| | | | | rs2893777 |
| | | | | rs7084955 |
| | | | | rs7085387 |
| | | | | rs7073985 |
| | | | | rs7077653 |
| | | | | rs16912268 |
| | | | | rs10826193 |
| | | | | rs2114562 |
| | | | | rs2114563 |
| | | | | rs1013473 |
| | | | | rs2128909 |
| | | | | rs1030432 |
| | | | | rs10509091 |
| | | | | rs7077537 |
| | | | | rs4948301 |
| | | | | rs1125139 |
| | | | | rs1896248 |
| | | | | rs2114561 |
| rs584762 | INTERGENIC | PAX6 | 31249 | rs585972 |
| | | | | rs604518 |
| | | | | rs685428 |
| | | | | rs624732 |
| | | | | rs761453 |
| | | | | rs2440250 |
| | | | | rs594462 |
| | | | | rs687947 |
| | | | | rs2473858 |
| | | | | rs677874 |
| rs761453 | INTERGENIC | PAX6 | 38491 | rs585972 |
| | | | | rs604518 |
| | | | | rs685428 |
| | | | | rs624732 |
| | | | | rs584762 |
| | | | | rs2440250 |
| | | | | rs594462 |
| | | | | rs687947 |
| | | | | rs2473858 |
| | | | | rs677874 |
| rs6035712 | INTERGENIC | C20orf74 | 132505 | rs6047077 |
| | | | | rs6035711 |
| rs6868846 | INTERGENIC | hsa-mir-582 | 129225 | rs256353 |
| | | | | rs173945 |
| | | | | rs256348 |
| | | | | rs159608 |
| rs554710 | UPSTREAM | SURF6 | −15721 | |
| rs1013120 | INTERGENIC | HS3ST3A1 | 39975 | rs968026 |
| | | | | rs11078172 |
| | | | | rs12952261 |
| rs649867 | INTRONIC | Q8N7E7_HUMAN | 0 | |
| rs6072317 | INTRONIC | ZHX3 | 0 | rs6072268 |
| | | | | rs17264110 |
| | | | | rs6072269 |
| | | | | rs6072275 |
| | | | | rs6072286 |
| | | | | rs2228246 |
| | | | | rs6072299 |
| | | | | rs6072300 |
| | | | | rs6072302 |
| | | | | rs2664537 |
| | | | | rs7261917 |
| | | | | rs6065325 |
| | | | | rs6065326 |
| | | | | rs6072320 |
| | | | | rs6072321 |
| | | | | rs6072322 |
| | | | | rs8121001 |
| | | | | rs17181845 |
| | | | | rs6072338 |
| rs4795069 | INTERGENIC | SLFN5 | 14839 | |
| rs13403584 | INTERGENIC | CCDC141 | 92384 | rs10171173 |
| | | | | rs2200826 |
| | | | | rs10497528 |
| | | | | rs7574599 |
| | | | | rs6747725 |
| | | | | rs924800 |
| | | | | rs10930847 |
| | | | | rs1847420 |
| | | | | rs4894072 |
| rs6948196 | INTRONIC | RHEB | 0 | rs17713386 |
| | | | | rs4726029 |
| | | | | rs875588 |
| | | | | rs1109089 |
| | | | | rs7805967 |
| | | | | rs17713697 |
| | | | | rs2074998 |
| | | | | rs2074997 |
| | | | | rs17713890 |
| | | | | rs2299961 |
| | | | | rs965118 |
| | | | | rs3753151 |
| | | | | rs2299965 |
| | | | | rs2284264 |
| | | | | rs11772458 |
| | | | | rs2299967 |
| | | | | rs3789817 |
| | | | | rs758666 |
| | | | | rs736645 |
| | | | | rs4298422 |
| | | | | rs2374261 |
| | | | | rs12112989 |
| | | | | rs6980020 |
| | | | | rs12112134 |
| | | | | rs6943752 |
| | | | | rs7794922 |
| rs2662090 | INTRONIC | SRGAP3 | 0 | rs2670000 |
| rs7685314 | INTERGENIC | LOC728856 | −28752 | rs6820247 |
| | | | | rs7437007 |
| | | | | rs10866319 |
| rs2074997 | INTRONIC | RHEB | 0 | rs17713386 |
| | | | | rs4726029 |
| | | | | rs875588 |
| | | | | rs1109089 |
| | | | | rs7805967 |
| | | | | rs17713697 |
| | | | | rs2074998 |
| | | | | rs17713890 |
| | | | | rs2299961 |
| | | | | rs965118 |
| | | | | rs3753151 |
| | | | | rs2299965 |
| | | | | rs2284264 |
| | | | | rs11772458 |
| | | | | rs2299967 |
| | | | | rs3789817 |
| | | | | rs758666 |
| | | | | rs736645 |
| | | | | rs4298422 |
| | | | | rs2374261 |
| | | | | rs12112989 |
| | | | | rs6980020 |
| | | | | rs12112134 |
| | | | | rs6948196 |
| | | | | rs6943752 |
| | | | | rs7794922 |
| rs6500498 | 3PRIME_UTR | Q8N7R2_HUMAN | 0 | rs7185630 |
| | | | | rs2242166 |
| | | | | rs2242164 |
| | | | | rs6500497 |
| rs1204798 | INTRONIC | NT5DC1 | 0 | rs1204842 |
| | | | | rs1204843 |
| | | | | rs742930 |
| | | | | rs7770203 |
| | | | | rs1211388 |
| | | | | rs1204851 |
| | | | | rs1209221 |
| | | | | rs1204782 |
| | | | | rs1204783 |
| | | | | rs1204784 |
| | | | | rs1204785 |
| | | | | rs1204786 |
| | | | | rs1204788 |
| | | | | rs1204789 |
| | | | | rs1204794 |
| | | | | rs1204797 |
| | | | | rs1204799 |
| | | | | rs1204800 |
| | | | | rs1204801 |
| | | | | rs1204802 |
| | | | | rs1209223 |
| | | | | rs1204807 |
| | | | | rs1204814 |
| | | | | rs926829 |
| | | | | rs1204817 |
| | | | | rs1204818 |
| rs12143647 | INTRONIC | COG2 | 0 | rs4846999 |
| | | | | rs12564388 |
| | | | | rs1887492 |
| | | | | rs11122569 |
| | | | | rs11122570 |
| | | | | rs3736983 |
| | | | | rs12041242 |
| | | | | rs12045973 |
| rs3801033 | INTRONIC | ZNF12 | 0 | rs3801034 |
| | | | | rs7798471 |
| rs10997044 | INTRONIC | CTNNA3 | 0 | rs1876334 |
| | | | | rs10822782 |
| | | | | rs10822783 |
| | | | | rs1911479 |
| | | | | rs10997021 |
| | | | | rs10822784 |
| | | | | rs4609495 |
| | | | | rs4745897 |
| | | | | rs10762045 |
| | | | | rs10997032 |
| | | | | rs12763572 |
| | | | | rs10822791 |
| | | | | rs10762046 |
| | | | | rs10822792 |
| | | | | rs9804181 |
| | | | | rs10997035 |
| | | | | rs10997037 |
| | | | | rs10822794 |
| | | | | rs10762051 |
| | | | | rs12357109 |
| | | | | rs1911485 |
| | | | | rs1911486 |
| | | | | rs10997048 |
| | | | | rs10822801 |
| | | | | rs4523585 |
| rs7350731 | INTERGENIC | N/A | −9 | |
| rs9323737 | INTERGENIC | N/A | −9 | rs1958438 |
| | | | | rs10139321 |
| | | | | rs10132490 |
| | | | | rs10143458 |
| | | | | rs9671273 |
| | | | | rs1952534 |
| | | | | rs1958436 |
| | | | | rs11159605 |
| | | | | rs7359138 |
| | | | | rs1958435 |
| | | | | rs1958434 |
| | | | | rs1958433 |
| | | | | rs2149684 |
| | | | | rs2183203 |
| | | | | rs1958432 |
| | | | | rs2149683 |
| | | | | rs8010141 |
| | | | | rs1958430 |
| | | | | rs1958429 |
| | | | | rs1958428 |
| | | | | rs1958426 |
| | | | | rs1958425 |
| | | | | rs1958424 |
| | | | | rs1958423 |
| | | | | rs1958422 |
| | | | | rs10132081 |
| | | | | rs10132130 |
| | | | | rs10147226 |
| | | | | rs1958421 |
| | | | | rs1958420 |
| | | | | rs10873350 |
| | | | | rs10140080 |
| | | | | rs4545730 |
| | | | | rs1952530 |
| | | | | rs1952529 |
| | | | | rs12431659 |
| | | | | rs12434622 |
| | | | | rs10141403 |
| | | | | rs9323738 |
| | | | | rs9323739 |
| | | | | rs1952528 |
| | | | | rs1958418 |
| | | | | rs877800 |
| | | | | rs2372550 |
| | | | | rs1952545 |
| | | | | rs1952544 |
| | | | | rs10747302 |
| | | | | rs4899838 |
| | | | | rs11159610 |
| | | | | rs11159611 |
| | | | | rs7494095 |
| | | | | rs10132137 |
| | | | | rs4899839 |
| | | | | rs10135095 |
| | | | | rs2888357 |
| | | | | rs4143911 |
| | | | | rs1958458 |
| | | | | rs2372553 |
| | | | | rs10147836 |
| | | | | rs10139812 |
| | | | | rs1958456 |
| | | | | rs1958455 |
| | | | | rs2372523 |
| | | | | rs2372524 |
| | | | | rs10143348 |
| | | | | rs9323740 |
| | | | | rs10130591 |
| | | | | rs1958449 |
| rs4881394 | INTERGENIC | AKR1C3 | −22872 | rs4525119 |
| | | | | rs4881388 |
| | | | | rs10795234 |
| | | | | rs7074522 |
| | | | | rs10795241 |
| | | | | rs2096422 |
| rs4799159 | INTERGENIC | SALL3 | −459812 | rs185750 |
| | | | | rs2578210 |
| | | | | rs12456117 |
| | | | | rs7506508 |
| | | | | rs9952440 |
| rs356999 | INTERGENIC | BCL11A | 30951 | rs357003 |
| | | | | rs357002 |
| | | | | rs356998 |
| rs2270101 | INTERGENIC | FAM126A | −47256 | rs1001027 |
| | | | | rs1001026 |
| | | | | rs13227316 |
| | | | | rs11765548 |
| | | | | rs12700402 |
| rs618670 | INTERGENIC | N/A | −9 | |
| rs7788668 | DOWNSTREAM | FAM126A | −2622 | rs2286497 |
| | | | | rs2033670 |
| | | | | rs13227654 |
| | | | | rs11561822 |
| | | | | rs11764386 |
| | | | | rs10488277 |
| | | | | rs2286493 |
| | | | | rs2286491 |
| | | | | rs2286490 |
| | | | | rs11772725 |
| | | | | rs11772749 |
| | | | | rs11764613 |
| | | | | rs11768060 |
| | | | | rs17147527 |
| | | | | rs17147529 |
| | | | | rs11762669 |
| | | | | rs13225964 |
| | | | | rs11771543 |
| | | | | rs17370052 |
| | | | | rs9647996 |
| | | | | rs12674362 |
| | | | | rs13230424 |
| | | | | rs10950935 |
| rs902923 | INTRONIC | ROR2 | 0 | rs10991949 |
| | | | | rs768056 |
| | | | | rs1412466 |
| | | | | rs16907585 |
| | | | | rs7855644 |
| | | | | rs1492679 |
| | | | | rs2131301 |
| | | | | rs9409427 |
| rs9332172 | INTRONIC | CYP2C9 | 0 | rs17521564 |
| | | | | rs7893293 |
| | | | | rs2860905 |
| | | | | rs4086116 |
| | | | | rs4917639 |
| | | | | rs1934963 |
| | | | | rs4918797 |
| rs6873640 | INTERGENIC | ZNF608 | −56628 | |
| | | | | rs7725329 |
| rs9302822 | INTERGENIC | Q8N9J9_HUMAN | 114376 | rs17539244 |
| | | | | rs1019191 |
| | | | | rs17442566 |
| | | | | rs17442866 |
| | | | | rs8050684 |
| | | | | rs17540392 |
| rs4810622 | INTRONIC | ZMYND8 | 0 | rs4809630 |
| | | | | rs13038759 |
| | | | | rs761021 |
| | | | | rs6124987 |
| | | | | rs3803941 |
| | | | | rs6124990 |
| rs3753151 | INTRONIC | RHEB | 0 | rs17713386 |
| | | | | rs4726029 |
| | | | | rs875588 |
| | | | | rs1109089 |
| | | | | rs7805967 |
| | | | | rs2074998 |
| | | | | rs2074997 |
| | | | | rs17713890 |
| | | | | rs2299961 |
| | | | | rs2299962 |
| | | | | rs2299965 |
| | | | | rs2284264 |
| | | | | rs2299967 |
| | | | | rs3789817 |
| | | | | rs758666 |
| | | | | rs736645 |
| | | | | rs4298422 |
| | | | | rs2374261 |
| | | | | rs12112989 |
| | | | | rs6980020 |
| | | | | rs12112134 |
| | | | | rs6948196 |
| rs10490832 | SYNONYMOUS_CODING | CNTN3 | 0 | |
| rs1545384 | INTRONIC | CNTN3 | 0 | rs1809043 |
| | | | | rs2197742 |
| rs1884641 | INTERGENIC | C20orf75 | 5233 | rs6117052 |
| | | | | rs6076925 |
| rs1958438 | INTERGENIC | N/A | −9 | rs10139321 |
| | | | | rs10132490 |
| | | | | rs10143458 |
| | | | | rs9671273 |
| | | | | rs1952534 |
| | | | | rs1958436 |
| | | | | rs11159605 |
| | | | | rs7359138 |
| | | | | rs1958435 |
| | | | | rs1958434 |
| | | | | rs1958433 |
| | | | | rs2149684 |
| | | | | rs2183203 |
| | | | | rs1958432 |
| | | | | rs2149683 |
| | | | | rs8010141 |
| | | | | rs1958430 |
| | | | | rs1958429 |
| | | | | rs1958428 |
| | | | | rs1958426 |
| | | | | rs1958425 |
| | | | | rs1958424 |
| | | | | rs1958423 |
| | | | | rs1958422 |
| | | | | rs10132081 |
| | | | | rs10132130 |
| | | | | rs10147226 |
| | | | | rs1958421 |
| | | | | rs1958420 |
| | | | | rs10873350 |
| | | | | rs10140080 |
| | | | | rs9323737 |
| | | | | rs4545730 |
| | | | | rs1952530 |
| | | | | rs1952529 |
| | | | | rs12431659 |
| | | | | rs12434622 |
| | | | | rs10141403 |
| | | | | rs9323738 |
| | | | | rs9323739 |
| | | | | rs1952528 |
| | | | | rs1958418 |
| | | | | rs877800 |
| | | | | rs2372550 |
| | | | | rs1952545 |
| | | | | rs1952544 |
| | | | | rs10747302 |
| | | | | rs4899838 |
| | | | | rs11159610 |
| | | | | rs11159611 |
| | | | | rs7494095 |
| | | | | rs10132137 |
| | | | | rs4899839 |
| | | | | rs10135095 |
| | | | | rs2888357 |
| | | | | rs4143911 |
| | | | | rs1958458 |
| | | | | rs2372553 |
| | | | | rs10147836 |
| | | | | rs10139812 |
| | | | | rs1958456 |
| | | | | rs1958455 |
| | | | | rs2372523 |
| | | | | rs2372524 |
| | | | | rs10143348 |
| | | | | rs9323740 |
| | | | | rs10130591 |
| | | | | rs12894765 |
| | | | | rs7157077 |
| | | | | rs4904138 |
| | | | | rs1952541 |
| | | | | rs1958451 |
| | | | | rs12882544 |
| | | | | rs1958449 |
| | | | | rs1952538 |
| rs4917639 | INTRONIC | CYP2C9 | 0 | rs17521564 |
| | | | | rs7893293 |
| | | | | rs2860905 |
| | | | | rs4086116 |
| | | | | rs9332172 |
| | | | | rs1934963 |
| | | | | rs4918797 |
| rs2279103 | NON_SYNONYMOUS_CODING | CTDP1 | 0 | rs3809939 |
| | | | | rs8098133 |
| | | | | rs3859315 |
| | | | | rs3859316 |
| | | | | rs898619 |
| | | | | rs3809936 |
| | | | | rs3786235 |
| | | | | rs8087647 |
| | | | | rs8084175 |
| | | | | rs9953991 |
| | | | | rs9946977 |
| | | | | rs12605690 |
| | | | | rs554659 |
| | | | | rs551017 |
| | | | | rs665138 |
| | | | | rs523386 |
| | | | | rs652717 |
| | | | | rs496036 |
| | | | | rs576937 |
| | | | | rs621490 |
| rs7307064 | INTRONIC | RECQL | 0 | rs7307519 |
| | | | | rs7976409 |
| | | | | rs7976415 |
| | | | | rs7956315 |
| | | | | rs17627102 |
| | | | | rs7310464 |
| | | | | rs1061626 |
| rs6793017 | INTRONIC | CNTN4 | 0 | rs6763008 |
| | | | | rs6774320 |
| rs523386 | INTERGENIC | Q6ZVY3_HUMAN | 10572 | rs3809939 |
| | | | | rs8098133 |
| | | | | rs3859315 |
| | | | | rs3859316 |
| | | | | rs898619 |
| | | | | rs3809936 |
| | | | | rs3786235 |
| | | | | rs8087647 |
| | | | | rs2279103 |
| | | | | rs8084175 |
| | | | | rs9953991 |
| | | | | rs9946977 |
| | | | | rs12605690 |
| | | | | rs554659 |
| | | | | rs551017 |
| | | | | rs665138 |
| | | | | rs652717 |
| | | | | rs496036 |
| | | | | rs576937 |
| | | | | rs621490 |
| rs7719325 | INTERGENIC | ZNF608 | −493552 | rs17151230 |
| | | | | rs17151248 |
| rs1984151 | INTERGENIC | EPHA7 | 399827 | rs2325528 |
| | | | | rs9452447 |
| | | | | rs9294577 |
| | | | | rs2152543 |
| | | | | rs4707810 |
| | | | | rs6904831 |
| | | | | rs2000361 |
| | | | | rs7765999 |
| | | | | rs6925802 |
| | | | | rs9294582 |
| | | | | rs7752145 |
| | | | | rs1238901 |
| | | | | rs1219036 |
| | | | | rs1219037 |
| | | | | rs2248260 |
| | | | | rs2801552 |
| | | | | rs2801556 |
| | | | | rs2633619 |
| | | | | rs2801559 |
| | | | | rs1601856 |
| rs1454333 | INTERGENIC | TMSL3 | 82867 | rs17017774 |
| | | | | rs1377917 |
| rs11250017 | INTERGENIC | Q6ZVI4_HUMAN | −12575 | rs10095339 |
| | | | | rs10103466 |
| rs7146332 | INTERGENIC | LRFN5 | −298872 | rs8005889 |
| | | | | rs4128092 |
| | | | | rs7161171 |
| | | | | rs6572078 |
| | | | | rs3866730 |
| | | | | rs6572079 |
| | | | | rs8019332 |
| | | | | rs4128087 |
| | | | | rs11628088 |
| | | | | rs12892392 |
| | | | | rs1431032 |
| rs17175096 | INTERGENIC | N/A | −9 | rs12960630 |
| | | | | rs17260406 |
| | | | | rs17074095 |
| rs720733 | INTERGENIC | PCDH18 | −443488 | rs6825957 |
| | | | | rs1914603 |
| | | | | rs10857200 |
| | | | | rs10857201 |
| | | | | rs6825255 |
| | | | | rs6853959 |
| | | | | rs4864386 |
| rs2299965 | INTRONIC | RHEB | 0 | rs17713386 |
| | | | | rs4726029 |
| | | | | rs875588 |
| | | | | rs1109089 |
| | | | | rs7805967 |
| | | | | rs17713697 |
| | | | | rs2074998 |
| | | | | rs2074997 |
| | | | | rs17713890 |
| | | | | rs2299961 |
| | | | | rs965118 |
| | | | | rs3753151 |
| | | | | rs2284264 |
| | | | | rs11772458 |
| | | | | rs2299967 |
| | | | | rs3789817 |
| | | | | rs758666 |
| | | | | rs736645 |
| | | | | rs4298422 |
| | | | | rs2374261 |
| | | | | rs12112989 |
| | | | | rs6980020 |
| | | | | rs12112134 |
| | | | | rs6948196 |
| | | | | rs6943752 |
| | | | | rs7794922 |
| rs2299967 | INTRONIC | RHEB | 0 | rs17713386 |
| | | | | rs4726029 |
| | | | | rs875588 |
| | | | | rs1109089 |
| | | | | rs7805967 |
| | | | | rs17713697 |
| | | | | rs2074998 |
| | | | | rs2074997 |
| | | | | rs17713890 |
| | | | | rs2299961 |
| | | | | rs965118 |
| | | | | rs3753151 |
| | | | | rs2299965 |
| | | | | rs2284264 |
| | | | | rs11772458 |
| | | | | rs3789817 |
| | | | | rs758666 |
| | | | | rs736645 |
| | | | | rs4298422 |
| | | | | rs2374261 |
| | | | | rs12112989 |
| | | | | rs6980020 |
| | | | | rs12112134 |
| | | | | rs6948196 |
| | | | | rs6943752 |
| | | | | rs7794922 |
| rs7550277 | INTRONIC | RGS7 | 0 | rs11589937 |
| | | | | rs1110128 |
| | | | | rs6702310 |
| | | | | rs11583779 |
| | | | | rs11584248 |
| | | | | rs1996805 |
| | | | | rs11587158 |
| | | | | rs11584771 |
| | | | | rs10926454 |
| | | | | rs7539742 |
| | | | | rs6429256 |
| | | | | rs6684760 |
| rs635546 | INTERGENIC | L3MBTL4 | −16980 | |
| rs301191 | INTERGENIC | N/A | −9 | rs177295 |
| | | | | rs301193 |
| | | | | rs2062246 |
| | | | | rs4857681 |
| | | | | rs4857686 |
| | | | | rs11710431 |
| | | | | rs4857698 |
| | | | | rs13091297 |
| | | | | rs9798968 |
| | | | | rs11922088 |
| rs301193 | INTERGENIC | N/A | −9 | rs177295 |
| | | | | rs301191 |
| | | | | rs2062246 |
| | | | | rs4857681 |
| | | | | rs4857686 |
| | | | | rs11710431 |
| | | | | rs4857698 |
| | | | | rs13091297 |
| | | | | rs9798968 |
| | | | | rs11922088 |
| rs12583395 | INTERGENIC | NBEA | −296678 | |
| rs4388301 | INTERGENIC | TNFAIP3 | 77933 | |
| rs4939517 | INTRONIC | CYBASC3 | 0 | rs7111608 |
| | | | | rs11230659 |
| | | | | rs6591651 |
| | | | | rs1037448 |
| | | | | rs10750955 |
| | | | | rs3741265 |
| | | | | rs10897158 |
| | | | | rs921635 |
| | | | | rs3018727 |
| | | | | rs3019198 |
| | | | | rs2860519 |
| | | | | rs3809083 |
| | | | | rs2943807 |
| | | | | rs2943806 |
| | | | | rs896831 |
| | | | | rs879647 |
| | | | | rs3017602 |
| | | | | rs2943805 |
| | | | | rs17702 |
| | | | | rs1377456 |
| | | | | rs720888 |
| | | | | rs720891 |
| | | | | rs6591654 |
| | | | | rs6591655 |
| | | | | rs729404 |
| | | | | rs729347 |
| | | | | rs3018729 |
| | | | | rs2924436 |
| | | | | rs11230701 |
| | | | | rs3019186 |
| | | | | rs896829 |
| | | | | rs748902 |
| | | | | rs730338 |
| | | | | rs2943800 |
| | | | | rs2957860 |
| | | | | rs2924441 |
| | | | | rs2924446 |
| | | | | rs3017605 |
| | | | | rs2100388 |
| rs1037448 | INTRONIC | TMEM138 | 0 | rs7111608 |
| | | | | rs11230659 |
| | | | | rs6591651 |
| | | | | rs4939517 |
| | | | | rs10750955 |
| | | | | rs3741265 |
| | | | | rs10897158 |
| | | | | rs921635 |
| | | | | rs3018727 |
| | | | | rs3019198 |
| | | | | rs2860519 |
| | | | | rs3809083 |
| | | | | rs2943807 |
| | | | | rs2943806 |
| | | | | rs896831 |
| | | | | rs879647 |
| | | | | rs3017602 |
| | | | | rs2943805 |
| | | | | rs17702 |
| | | | | rs1377456 |
| | | | | rs720888 |
| | | | | rs720891 |
| | | | | rs6591654 |
| | | | | rs6591655 |
| | | | | rs729404 |
| | | | | rs729347 |
| | | | | rs3018729 |
| | | | | rs2924436 |
| | | | | rs11230701 |
| | | | | rs3019186 |
| | | | | rs896829 |
| | | | | rs748902 |
| | | | | rs730338 |
| | | | | rs2943800 |
| | | | | rs2957860 |
| | | | | rs2924441 |
| | | | | rs2924446 |
| | | | | rs3017605 |
| | | | | rs2100388 |
| rs2348427 | INTRONIC | ENPEP | 0 | rs1126483 |
| | | | | rs12503640 |
| | | | | rs2881913 |
| | | | | rs2348429 |
| | | | | rs17551888 |
| | | | | rs6842486 |
| | | | | rs10015807 |
| | | | | rs12506732 |
| | | | | rs2348431 |
| | | | | rs6813802 |
| | | | | rs3796889 |
| | | | | rs3796888 |
| | | | | rs2348433 |
| | | | | rs1448808 |
| rs1891877 | INTRONIC | PIP4K2A | 0 | |
| rs8095186 | INTERGENIC | PIK3C3 | 251736 | rs346457 |
| rs9480684 | INTRONIC | AIM1 | 0 | rs1770728 |
| | | | | rs1770731 |
| | | | | rs1770732 |
| | | | | rs1676016 |
| | | | | rs2297971 |
| | | | | rs2066202 |
| | | | | rs2054366 |
| | | | | rs2615206 |
| | | | | rs2615207 |
| | | | | rs2642469 |
| | | | | rs1037955 |
| | | | | rs965347 |
| | | | | rs4946764 |
| | | | | rs9486398 |
| | | | | rs9486399 |
| | | | | rs9480685 |
| | | | | rs9486403 |
| | | | | rs9480686 |
| | | | | rs9486404 |
| | | | | rs3747790 |
| | | | | rs13319 |
| | | | | rs9320182 |
| | | | | rs9320183 |
| | | | | rs9486410 |
| | | | | rs9486411 |
| | | | | rs9486414 |
| | | | | rs9486415 |
| | | | | rs6926307 |
| | | | | rs6926670 |
| | | | | rs6926833 |
| | | | | rs6907164 |
| | | | | rs17067368 |
| | | | | rs9480689 |
| | | | | rs4523123 |
| | | | | rs7741101 |
| | | | | rs7776287 |
| | | | | rs7776294 |
| | | | | rs12523774 |
| | | | | rs6903754 |
| | | | | rs4945759 |
| | | | | rs9689006 |
| | | | | rs7770930 |

| | Tagged_SNP | |
|---|---|---|
| SNP | type | Tagged_SNP_r2 |
| rs1630535 | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 0.87 |
| rs4724701 | INTRONIC | 0.87 |
| | INTRONIC | 0.87 |
| | INTRONIC | 0.9 |
| | INTRONIC | 1 |
| | INTRONIC | 0.96 |
| | INTRONIC | 0.96 |
| | INTRONIC | 0.96 |
| | INTRONIC | 1 |
| rs1265235 | INTERGENIC | 1 |
| | INTERGENIC | 1 |
| | INTERGENIC | 0.84 |
| rs2414660 | INTERGENIC | 1 |
| | INTERGENIC | 0.82 |
| | INTERGENIC | 0.92 |
| | INTERGENIC | 0.91 |
| | INTERGENIC | 0.85 |
| | INTERGENIC | 0.9 |
| | INTERGENIC | 0.88 |
| | INTERGENIC | 0.92 |
| | INTERGENIC | 0.84 |
| rs12589623 | INTERGENIC | 0.85 |
| | INTERGENIC | 0.85 |
| | INTERGENIC | 1 |
| | INTERGENIC | 0.95 |
| rs279553 | 5PRIME_UTR | 0.89 |
| | 3PRIME_UTR | 1 |
| | INTRONIC | 1 |
| rs279542 | 5PRIME_UTR | 0.89 |
| | SYNONYMOUS_CODING | 1 |
| | INTRONIC | 1 |
| rs583338 | INTERGENIC | 0.9 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| rs2774089 | INTERGENIC | 0.97 |
| | INTERGENIC | 0.97 |
| | INTERGENIC | 0.97 |
| | INTERGENIC | 0.97 |
| rs1421780 | | |
| rs4907674 | | |
| rs7676106 | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 0.83 |
| | INTERGENIC | 0.83 |
| rs9949324 | INTERGENIC | 1 |
| | INTERGENIC | 1 |
| | INTERGENIC | 0.92 |
| | INTERGENIC | 1 |
| | INTERGENIC | 1 |
| rs7704939 | | |
| rs4518023 | INTERGENIC | 1 |
| | INTERGENIC | 1 |
| rs4544214 | INTRONIC | 0.81 |
| | INTRONIC | 1 |
| | UPSTREAM | 1 |
| rs6072343 | INTERGENIC | 0.92 |
| | INTERGENIC | 0.92 |
| | INTRONIC | 0.81 |
| rs965118 | DOWNSTREAM | 0.97 |
| | INTRONIC | 0.97 |
| | INTRONIC | 0.97 |
| | INTRONIC | 0.96 |
| | INTRONIC | 0.87 |
| | INTRONIC | 0.97 |
| | INTRONIC | 0.97 |
| | INTRONIC | 0.97 |
| | INTRONIC | 0.97 |
| | INTRONIC | 0.97 |
| | INTRONIC | 1 |
| | INTRONIC | 0.97 |
| | INTRONIC | 0.97 |
| | INTRONIC | 0.97 |
| | INTRONIC | 0.97 |
| | INTRONIC | 0.96 |
| | INTRONIC | 0.97 |
| | INTRONIC | 0.97 |
| | INTRONIC | 0.97 |
| | INTRONIC | 0.97 |
| | INTRONIC | 0.97 |
| | INTRONIC | 0.87 |
| | UPSTREAM | 0.81 |
| rs1109089 | DOWNSTREAM | 1 |
| | INTRONIC | 1 |
| | INTRONIC | 1 |
| | INTRONIC | 0.83 |
| | INTRONIC | 0.9 |
| | INTRONIC | 1 |
| | INTRONIC | 1 |
| | INTRONIC | 1 |
| | INTRONIC | 0.83 |
| | INTRONIC | 0.96 |
| | INTRONIC | 0.83 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 0.89 |
| | UPSTREAM | 0.86 |
| rs2980872 | INTERGENIC | 0.93 |
| rs12142266 | | |
| rs9877859 | INTRONIC | 0.84 |
| rs2025949 | INTRONIC | 0.91 |
| | INTRONIC | 0.94 |
| | INTRONIC | 0.9 |
| | INTRONIC | 0.9 |
| | INTRONIC | 0.94 |
| | INTRONIC | 0.95 |
| | INTRONIC | 1 |
| | INTRONIC | 1 |
| | INTRONIC | 0.95 |
| | INTRONIC | 0.95 |
| rs2070439 | | |
| rs12617566 | | |
| rs9589698 | INTRONIC | 1 |
| | INTRONIC | 1 |
| | INTRONIC | 1 |
| | INTRONIC | 1 |
| | INTRONIC | 1 |
| | INTRONIC | 0.91 |
| | INTRONIC | 0.9 |
| | INTRONIC | 0.91 |
| | INTRONIC | 0.95 |
| | INTRONIC | 0.89 |
| rs1958421 | INTERGENIC | 0.92 |
| | INTERGENIC | 0.92 |
| | INTERGENIC | 0.89 |
| | INTERGENIC | 0.91 |
| | INTERGENIC | 0.92 |
| | INTERGENIC | 0.92 |
| | INTERGENIC | 0.91 |
| | INTERGENIC | 0.95 |
| | INTERGENIC | 0.8 |
| | INTERGENIC | 0.92 |
| | INTERGENIC | 0.91 |
| | INTERGENIC | 0.92 |
| | INTERGENIC | 0.87 |
| | INTERGENIC | 0.91 |
| | INTERGENIC | 0.91 |
| | INTERGENIC | 0.92 |
| | INTERGENIC | 0.92 |
| | INTERGENIC | 0.92 |
| | INTERGENIC | 0.92 |
| | INTERGENIC | 0.92 |
| | INTERGENIC | 1 |
| | INTERGENIC | 0.92 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 0.92 |
| | INTERGENIC | 1 |
| | INTERGENIC | 1 |
| | INTERGENIC | 0.91 |
| | INTERGENIC | 1 |
| | INTERGENIC | 1 |
| | INTERGENIC | 1 |
| | INTERGENIC | 0.92 |
| | INTERGENIC | 0.9 |
| | INTERGENIC | 0.92 |
| | INTERGENIC | 0.92 |
| | INTERGENIC | 0.92 |
| | INTERGENIC | 0.92 |
| | INTERGENIC | 0.92 |
| | INTERGENIC | 0.92 |
| | INTERGENIC | 0.92 |
| | INTERGENIC | 0.91 |
| | INTERGENIC | 0.92 |
| | INTERGENIC | 0.87 |
| | INTERGENIC | 0.91 |
| | INTERGENIC | 0.92 |
| | INTERGENIC | 0.92 |
| | INTERGENIC | 0.87 |
| | INTERGENIC | 0.92 |
| | INTERGENIC | 0.92 |
| | INTERGENIC | 0.82 |
| | INTERGENIC | 0.87 |
| | INTERGENIC | 0.87 |
| rs7645289 | INTERGENIC | 1 |
| | INTERGENIC | 1 |
| | INTERGENIC | 0.93 |
| | INTERGENIC | 0.94 |
| | INTERGENIC | 0.94 |
| | INTERGENIC | 0.84 |
| | INTERGENIC | 0.84 |
| rs2088138 | | |
| rs2228246 | | |
| rs2664537 | NON_SYNONYMOUS_CODING | 0.82 |
| | DOWNSTREAM | 0.93 |
| | 3PRIME_UTR | 0.82 |
| | INTRONIC | 0.82 |
| | INTRONIC | 0.82 |
| | INTRONIC | 0.82 |
| | INTRONIC | 0.94 |
| | INTRONIC | 0.94 |
| | INTRONIC | 0.82 |
| | INTRONIC | 0.82 |
| rs6500497 | 3PRIME_UTR | 0.87 |
| | 3PRIME_UTR | 0.87 |
| | UPSTREAM | 0.87 |
| | 3PRIME_UTR | 1 |
| rs2839178 | | |
| rs11258903 | INTRONIC | 1 |
| | INTRONIC | 1 |
| rs2244057 | INTERGENIC | 0.94 |
| | INTERGENIC | 0.95 |
| | INTERGENIC | 1 |
| | INTERGENIC | 0.84 |
| rs1459841 | | |
| rs7901463 | INTERGENIC | 1 |
| | INTERGENIC | 0.87 |
| | INTERGENIC | 0.87 |
| | INTERGENIC | 0.93 |
| | INTERGENIC | 0.87 |
| | INTERGENIC | 1 |
| | INTERGENIC | 0.87 |
| | INTERGENIC | 1 |
| | INTERGENIC | 1 |
| | INTERGENIC | 0.89 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 0.87 |
| | INTERGENIC | 1 |
| | INTERGENIC | 0.81 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 0.87 |
| | INTERGENIC | 0.92 |
| | INTERGENIC | 0.85 |
| | INTERGENIC | 0.9 |
| | INTERGENIC | 1 |
| | INTERGENIC | 1 |
| | INTERGENIC | 1 |
| | INTERGENIC | 1 |
| | INTERGENIC | 0.87 |
| | INTERGENIC | 0.86 |
| | INTERGENIC | 1 |
| rs584762 | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 0.95 |
| | INTERGENIC | 0.95 |
| | INTERGENIC | 0.9 |
| | INTERGENIC | 0.9 |
| rs761453 | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 0.95 |
| | INTERGENIC | 0.95 |
| | INTERGENIC | 0.9 |
| | INTERGENIC | 0.9 |
| rs6035712 | INTERGENIC | 0.91 |
| | INTERGENIC | 0.96 |
| rs6868846 | | |
| | INTERGENIC | 1 |
| | INTERGENIC | 1 |
| | INTERGENIC | 0.96 |
| | INTERGENIC | 0.96 |
| rs554710 | | |
| rs1013120 | INTERGENIC | 0.95 |
| | INTERGENIC | 1 |
| | INTERGENIC | 0.9 |
| rs649867 | | |
| rs6072317 | INTRONIC | 0.82 |
| | INTRONIC | 0.82 |
| | INTRONIC | 0.82 |
| | INTRONIC | 0.82 |
| | INTRONIC | 1 |
| | NON_SYNONYMOUS_CODING | 1 |
| | DOWNSTREAM | 1 |
| | 3PRIME_UTR | 0.94 |
| | 3PRIME_UTR | 1 |
| | 3PRIME_UTR | 0.82 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 0.88 |
| | UPSTREAM | 0.94 |
| | UPSTREAM | 0.94 |
| rs4795069 | | |
| rs13403584 | INTERGENIC | 0.86 |
| | INTERGENIC | 0.9 |
| | INTERGENIC | 0.81 |
| | INTERGENIC | 0.9 |
| | INTERGENIC | 0.9 |
| | INTERGENIC | 0.96 |
| | INTERGENIC | 0.96 |
| | INTERGENIC | 0.96 |
| | INTERGENIC | 0.96 |
| rs6948196 | DOWNSTREAM | 1 |
| | INTRONIC | 1 |
| | INTRONIC | 1 |
| | INTRONIC | 1 |
| | INTRONIC | 0.82 |
| | INTRONIC | 0.9 |
| | INTRONIC | 1 |
| | INTRONIC | 1 |
| | INTRONIC | 1 |
| | INTRONIC | 0.82 |
| | INTRONIC | 0.97 |
| | INTRONIC | 0.82 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 0.9 |
| | UPSTREAM | 0.84 |
| rs2662090 | INTRONIC | 0.97 |
| rs7685314 | INTERGENIC | 0.83 |
| | INTERGENIC | 1 |
| | INTERGENIC | 0.83 |
| rs2074997 | DOWNSTREAM | 1 |
| | INTRONIC | 1 |
| | INTRONIC | 1 |
| | INTRONIC | 1 |
| | INTRONIC | 0.81 |
| | INTRONIC | 0.9 |
| | INTRONIC | 1 |
| | INTRONIC | 1 |
| | INTRONIC | 0.81 |
| | INTRONIC | 0.97 |
| | INTRONIC | 0.81 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 0.9 |
| | UPSTREAM | 0.84 |
| rs6500498 | 3PRIME_UTR | 0.87 |
| | 3PRIME_UTR | 0.87 |
| | UPSTREAM | 0.87 |
| | 3PRIME_UTR | 1 |
| rs1204798 | INTRONIC | 1 |
| | INTRONIC | 1 |
| | INTRONIC | 1 |
| | INTRONIC | 1 |
| | INTRONIC | 0.83 |
| | INTRONIC | 1 |
| | INTRONIC | 1 |
| | INTRONIC | 1 |
| | INTRONIC | 1 |
| | INTRONIC | 0.88 |
| | INTRONIC | 0.83 |
| | INTRONIC | 0.94 |
| | INTRONIC | 1 |
| | INTRONIC | 0.88 |
| | INTRONIC | 0.83 |
| | INTRONIC | 0.88 |
| | INTRONIC | 0.83 |
| | INTRONIC | 1 |
| | INTRONIC | 1 |
| | INTRONIC | 0.82 |
| | INTRONIC | 1 |
| | INTRONIC | 1 |
| | INTRONIC | 1 |
| | INTRONIC | 1 |
| | INTRONIC | 0.83 |
| | INTRONIC | 0.82 |
| rs12143647 | INTRONIC | 0.81 |
| | INTRONIC | 0.81 |
| | INTRONIC | 0.82 |
| | INTRONIC | 0.86 |
| | INTRONIC | 0.82 |
| | INTRONIC | 0.82 |
| | INTRONIC | 0.84 |
| | INTRONIC | 0.84 |
| rs3801033 | INTRONIC | 0.85 |
| | INTRONIC | 0.8 |
| rs10997044 | INTRONIC | 0.84 |
| | INTRONIC | 0.95 |
| | INTRONIC | 0.95 |
| | INTRONIC | 0.95 |
| | INTRONIC | 0.95 |
| | INTRONIC | 0.95 |
| | INTRONIC | 0.95 |
| | INTRONIC | 0.95 |
| | INTRONIC | 0.95 |
| | INTRONIC | 0.95 |
| | INTRONIC | 0.95 |
| | INTRONIC | 0.91 |
| | INTRONIC | 0.91 |
| | INTRONIC | 0.95 |
| | INTRONIC | 0.95 |
| | INTRONIC | 0.95 |
| | INTRONIC | 0.95 |
| | INTRONIC | 0.95 |
| | INTRONIC | 0.91 |
| | INTRONIC | 0.95 |
| | INTRONIC | 0.81 |
| | INTRONIC | 1 |
| | INTRONIC | 1 |
| | INTRONIC | 0.95 |
| | INTRONIC | 0.95 |
| rs7350731 | | |
| rs9323737 | INTERGENIC | 0.92 |
| | INTERGENIC | 0.92 |
| | INTERGENIC | 0.89 |
| | INTERGENIC | 0.91 |
| | INTERGENIC | 0.92 |
| | INTERGENIC | 0.92 |
| | INTERGENIC | 0.91 |
| | INTERGENIC | 0.95 |
| | INTERGENIC | 0.8 |
| | INTERGENIC | 0.92 |
| | INTERGENIC | 0.91 |
| | INTERGENIC | 0.92 |
| | INTERGENIC | 0.87 |
| | INTERGENIC | 0.91 |
| | INTERGENIC | 0.91 |
| | INTERGENIC | 0.92 |
| | INTERGENIC | 0.92 |
| | INTERGENIC | 0.92 |
| | INTERGENIC | 0.92 |
| | INTERGENIC | 0.92 |
| | INTERGENIC | 1 |
| | INTERGENIC | 0.92 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 0.92 |
| | INTERGENIC | 1 |
| | INTERGENIC | 1 |
| | INTERGENIC | 0.91 |
| | INTERGENIC | 1 |
| | INTERGENIC | 1 |
| | INTERGENIC | 1 |
| | INTERGENIC | 0.92 |
| | INTERGENIC | 0.9 |
| | INTERGENIC | 0.92 |
| | INTERGENIC | 0.92 |
| | INTERGENIC | 0.92 |
| | INTERGENIC | 0.92 |
| | INTERGENIC | 0.92 |
| | INTERGENIC | 0.92 |
| | INTERGENIC | 0.92 |
| | INTERGENIC | 0.91 |
| | INTERGENIC | 0.92 |
| | INTERGENIC | 0.87 |
| | INTERGENIC | 0.91 |
| | INTERGENIC | 0.92 |
| | INTERGENIC | 0.92 |
| | INTERGENIC | 0.87 |
| | INTERGENIC | 0.92 |
| | INTERGENIC | 0.92 |
| | INTERGENIC | 0.82 |
| | INTERGENIC | 0.87 |
| | INTERGENIC | 0.87 |
| rs4881394 | INTERGENIC | 0.8 |
| | INTERGENIC | 0.8 |
| | INTERGENIC | 0.96 |
| | INTERGENIC | 0.96 |
| | INTERGENIC | 0.8 |
| | INTERGENIC | 0.85 |
| rs4799159 | INTERGENIC | 0.85 |
| | INTERGENIC | 0.85 |
| | INTERGENIC | 0.93 |
| | INTERGENIC | 0.93 |
| | INTERGENIC | 1 |
| rs356999 | INTERGENIC | 1 |
| | INTERGENIC | 1 |
| | INTERGENIC | 1 |
| rs2270101 | INTERGENIC | 0.96 |
| | INTERGENIC | 0.96 |
| | INTERGENIC | 0.96 |
| | INTERGENIC | 0.96 |
| | INTERGENIC | 0.96 |
| rs618670 | | |
| rs7788668 | INTERGENIC | 0.92 |
| | INTERGENIC | 0.92 |
| | INTERGENIC | 0.83 |
| | INTERGENIC | 0.96 |
| | DOWNSTREAM | 1 |
| | 3PRIME_UTR | 0.96 |
| | 3PRIME_UTR | 0.96 |
| | INTRONIC | 1 |
| | INTRONIC | 1 |
| | INTRONIC | 0.95 |
| | INTRONIC | 1 |
| | INTRONIC | 0.83 |
| | INTRONIC | 0.82 |
| | INTRONIC | 1 |
| | INTRONIC | 0.85 |
| | INTRONIC | 0.88 |
| | INTRONIC | 0.83 |
| | INTERGENIC | 0.96 |
| | INTERGENIC | 0.96 |
| | INTERGENIC | 0.96 |
| | INTERGENIC | 0.81 |
| | INTERGENIC | 0.84 |
| | INTERGENIC | 0.88 |
| rs902923 | INTERGENIC | 0.88 |
| | INTERGENIC | 0.94 |
| | INTERGENIC | 0.88 |
| | INTERGENIC | 0.89 |
| | INTERGENIC | 0.89 |
| | INTERGENIC | 1 |
| | DOWNSTREAM | 1 |
| | INTRONIC | 1 |
| rs9332172 | INTERGENIC | 1 |
| | INTERGENIC | 0.9 |
| | INTRONIC | 0.9 |
| | INTRONIC | 1 |
| | INTRONIC | 1 |
| | INTRONIC | 1 |
| | DOWNSTREAM | 1 |
| rs6873640 | INTERGENIC | 0.97 |
| rs9302822 | INTERGENIC | 0.84 |
| | INTERGENIC | 0.84 |
| | INTERGENIC | 0.84 |
| | INTERGENIC | 0.81 |
| | INTERGENIC | 0.81 |
| | INTERGENIC | 0.81 |
| rs4810622 | INTRONIC | 0.93 |
| | INTRONIC | 0.82 |
| | INTRONIC | 0.82 |
| | INTRONIC | 1 |
| | INTRONIC | 1 |
| | INTRONIC | 0.92 |
| rs3753151 | DOWNSTREAM | 0.84 |
| | INTRONIC | 0.82 |
| | INTRONIC | 0.82 |
| | INTRONIC | 0.83 |
| | INTRONIC | 1 |
| | INTRONIC | 0.84 |
| | INTRONIC | 0.81 |
| | INTRONIC | 0.82 |
| | INTRONIC | 1 |
| | INTRONIC | 0.9 |
| | INTRONIC | 0.82 |
| | INTRONIC | 0.82 |
| | INTRONIC | 0.84 |
| | INTRONIC | 0.82 |
| | INTRONIC | 0.82 |
| | INTRONIC | 0.82 |
| | INTRONIC | 0.84 |
| | INTRONIC | 0.82 |
| | INTRONIC | 0.82 |
| | INTRONIC | 0.82 |
| | INTRONIC | 0.81 |
| | INTRONIC | 0.82 |
| rs10490832 | | |
| rs1545384 | INTRONIC | 0.9 |
| | INTRONIC | 0.95 |
| rs1884641 | UPSTREAM | 1 |
| | INTERGENIC | 0.81 |
| rs1958438 | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 0.9 |
| | INTERGENIC | 1 |
| | INTERGENIC | 1 |
| | INTERGENIC | 1 |
| | INTERGENIC | 0.96 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 0.92 |
| | INTERGENIC | 1 |
| | INTERGENIC | 0.92 |
| | INTERGENIC | 0.92 |
| | INTERGENIC | 0.92 |
| | INTERGENIC | 0.92 |
| | INTERGENIC | 0.91 |
| | INTERGENIC | 0.91 |
| | INTERGENIC | 0.92 |
| | INTERGENIC | 0.92 |
| | INTERGENIC | 0.92 |
| | INTERGENIC | 0.92 |
| | INTERGENIC | 0.92 |
| | INTERGENIC | 0.92 |
| | INTERGENIC | 0.92 |
| | INTERGENIC | 0.92 |
| | INTERGENIC | 0.91 |
| | INTERGENIC | 0.95 |
| | INTERGENIC | 0.91 |
| | INTERGENIC | 0.92 |
| | INTERGENIC | 0.92 |
| | INTERGENIC | 0.84 |
| | INTERGENIC | 0.92 |
| | INTERGENIC | 0.91 |
| | INTERGENIC | 0.82 |
| | INTERGENIC | 0.92 |
| | INTERGENIC | 0.92 |
| | INTERGENIC | 0.95 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 0.95 |
| | INTERGENIC | 1 |
| | INTERGENIC | 1 |
| | INTERGENIC | 1 |
| | INTERGENIC | 0.95 |
| | INTERGENIC | 1 |
| | INTERGENIC | 1 |
| | INTERGENIC | 0.91 |
| | INTERGENIC | 0.95 |
| | INTERGENIC | 0.87 |
| | INTERGENIC | 0.87 |
| | INTERGENIC | 0.87 |
| | INTERGENIC | 0.87 |
| | INTERGENIC | 0.87 |
| | INTERGENIC | 0.87 |
| | INTERGENIC | 0.95 |
| | INTERGENIC | 0.87 |
| rs4917639 | INTERGENIC | 1 |
| | INTERGENIC | 0.91 |
| | INTRONIC | 0.91 |
| | INTRONIC | 1 |
| | INTRONIC | 1 |
| | INTRONIC | 1 |
| | DOWNSTREAM | 1 |
| rs2279103 | INTRONIC | 0.86 |
| | INTRONIC | 0.95 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 0.82 |
| | 3PRIME_UTR | 1 |
| | DOWNSTREAM | 0.82 |
| | INTERGENIC | 0.82 |
| | INTERGENIC | 0.82 |
| | INTERGENIC | 0.82 |
| | INTERGENIC | 1 |
| | INTERGENIC | 0.82 |
| | INTERGENIC | 1 |
| | INTERGENIC | 0.81 |
| | INTERGENIC | 0.95 |
| rs7307064 | INTRONIC | 1 |
| | INTRONIC | 1 |
| | INTRONIC | 1 |
| | INTRONIC | 1 |
| | INTRONIC | 0.85 |
| | INTRONIC | 0.91 |
| | 5PRIME_UTR | 0.86 |
| rs6793017 | INTRONIC | 0.88 |
| | INTRONIC | 0.95 |
| rs523386 | INTRONIC | 0.86 |
| | INTRONIC | 0.95 |
| | INTRONIC | 1 |
| | INTRONIC | 1 |
| | INTRONIC | 1 |
| | INTRONIC | 1 |
| | INTRONIC | 1 |
| | INTRONIC | 1 |
| | NON_SYNONYMOUS_CODING | 1 |
| | INTRONIC | 1 |
| | INTRONIC | 0.82 |
| | 3PRIME_UTR | 1 |
| | DOWNSTREAM | 0.82 |
| | INTERGENIC | 0.82 |
| | INTERGENIC | 0.82 |
| | INTERGENIC | 0.82 |
| | INTERGENIC | 0.82 |
| | INTERGENIC | 1 |
| | INTERGENIC | 0.81 |
| | INTERGENIC | 0.95 |
| rs7719325 | INTERGENIC | 0.95 |
| | INTERGENIC | 1 |
| rs1984151 | INTERGENIC | 1 |
| | INTERGENIC | 1 |
| | INTERGENIC | 1 |
| | INTERGENIC | 1 |
| | INTERGENIC | 1 |
| | INTERGENIC | 0.96 |
| | INTERGENIC | 0.95 |
| | INTERGENIC | 0.85 |
| | INTERGENIC | 0.83 |
| | INTERGENIC | 0.85 |
| | INTERGENIC | 0.88 |
| | INTERGENIC | 0.85 |
| | INTERGENIC | 0.82 |
| | INTERGENIC | 0.81 |
| | INTERGENIC | 0.85 |
| | INTERGENIC | 0.82 |
| | INTERGENIC | 0.82 |
| | INTERGENIC | 0.82 |
| | INTERGENIC | 0.82 |
| | INTERGENIC | 0.82 |
| rs1454333 | INTERGENIC | 0.93 |
| | INTERGENIC | 0.93 |
| rs11250017 | DOWNSTREAM | 0.83 |
| | DOWNSTREAM | 0.96 |
| rs7146332 | INTERGENIC | 0.97 |
| | INTERGENIC | 0.96 |
| | INTERGENIC | 0.97 |
| | INTERGENIC | 0.88 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| rs17175096 | INTERGENIC | 0.89 |
| | INTERGENIC | 0.94 |
| | INTERGENIC | 1 |
| rs720733 | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| rs2299965 | DOWNSTREAM | 1 |
| | INTRONIC | 1 |
| | INTRONIC | 1 |
| | INTRONIC | 1 |
| | INTRONIC | 0.82 |
| | INTRONIC | 0.9 |
| | INTRONIC | 1 |
| | INTRONIC | 1 |
| | INTRONIC | 1 |
| | INTRONIC | 0.82 |
| | INTRONIC | 0.97 |
| | INTRONIC | 0.82 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 0.9 |
| | UPSTREAM | 0.84 |
| rs2299967 | DOWNSTREAM | 1 |
| | INTRONIC | 1 |
| | INTRONIC | 1 |
| | INTRONIC | 1 |
| | INTRONIC | 0.84 |
| | INTRONIC | 0.9 |
| | INTRONIC | 1 |
| | INTRONIC | 1 |
| | INTRONIC | 1 |
| | INTRONIC | 0.84 |
| | INTRONIC | 0.97 |
| | INTRONIC | 0.84 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 0.9 |
| | UPSTREAM | 0.84 |
| rs7550277 | INTRONIC | 0.86 |
| | INTRONIC | 0.86 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| rs635546 | | |
| rs301191 | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 0.86 |
| | INTERGENIC | 1 |
| rs301193 | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 1 |
| | INTERGENIC | 0.86 |
| | INTERGENIC | 1 |
| rs12583395 | | |
| rs4388301 | | |
| rs4939517 | INTRONIC | 1 |
| | INTRONIC | 1 |
| | INTRONIC | 1 |
| | INTRONIC | 1 |
| | UPSTREAM | 1 |
| | SYNONYMOUS_CODING | 0.94 |
| | INTRONIC | 0.94 |
| | INTRONIC | 0.94 |
| | INTRONIC | 0.94 |
| | SYNONYMOUS_CODING | 0.93 |
| | INTRONIC | 0.93 |
| | INTRONIC | 0.94 |
| | INTRONIC | 0.94 |
| | INTRONIC | 0.87 |
| | INTRONIC | 0.87 |
| | INTRONIC | 0.87 |
| | INTRONIC | 0.87 |
| | INTRONIC | 0.87 |
| | 3PRIME_UTR | 0.87 |
| | DOWNSTREAM | 0.87 |
| | DOWNSTREAM | 0.84 |
| | DOWNSTREAM | 0.87 |
| | DOWNSTREAM | 0.87 |
| | INTERGENIC | 0.87 |
| | INTERGENIC | 0.87 |
| | INTERGENIC | 0.87 |
| | INTERGENIC | 0.87 |
| | INTERGENIC | 0.87 |
| | INTERGENIC | 0.87 |
| | INTERGENIC | 0.87 |
| | INTERGENIC | 0.87 |
| | INTERGENIC | 0.94 |
| | INTERGENIC | 0.85 |
| | UPSTREAM | 0.94 |
| | UPSTREAM | 0.93 |
| | UPSTREAM | 0.94 |
| | DOWNSTREAM | 0.87 |
| | DOWNSTREAM | 0.81 |
| | INTERGENIC | 0.87 |
| rs1037448 | INTRONIC | 1 |
| | INTRONIC | 1 |
| | INTRONIC | 1 |
| | INTRONIC | 1 |
| | UPSTREAM | 1 |
| | SYNONYMOUS_CODING | 0.94 |
| | INTRONIC | 0.94 |
| | INTRONIC | 0.94 |
| | INTRONIC | 0.94 |
| | SYNONYMOUS_CODING | 0.94 |
| | INTRONIC | 0.94 |
| | INTRONIC | 0.94 |
| | INTRONIC | 0.94 |
| | INTRONIC | 0.87 |
| | INTRONIC | 0.88 |
| | INTRONIC | 0.87 |
| | INTRONIC | 0.88 |
| | INTRONIC | 0.88 |
| | 3PRIME_UTR | 0.87 |
| | DOWNSTREAM | 0.88 |
| | DOWNSTREAM | 0.84 |
| | DOWNSTREAM | 0.88 |
| | DOWNSTREAM | 0.88 |
| | INTERGENIC | 0.88 |
| | INTERGENIC | 0.88 |
| | INTERGENIC | 0.87 |
| | INTERGENIC | 0.88 |
| | INTERGENIC | 0.88 |
| | INTERGENIC | 0.88 |
| | INTERGENIC | 0.88 |
| | INTERGENIC | 0.88 |
| | INTERGENIC | 0.94 |
| | INTERGENIC | 0.86 |
| | UPSTREAM | 0.94 |
| | UPSTREAM | 0.94 |
| | UPSTREAM | 0.94 |
| | DOWNSTREAM | 0.88 |
| | DOWNSTREAM | 0.82 |
| | INTERGENIC | 0.88 |
| rs2348427 | NON_SYNONYMOUS_CODING | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 0.96 |
| | INTRONIC | 0.96 |
| | INTRONIC | 0.96 |
| | INTRONIC | 0.96 |
| | INTRONIC | 1 |
| | INTRONIC | 1 |
| | INTRONIC | 0.8 |
| rs1891877 | | |
| rs8095186 | INTERGENIC | 0.92 |
| rs9480684 | INTRONIC | 0.93 |
| | INTRONIC | 0.91 |
| | INTRONIC | 0.93 |
| | INTRONIC | 0.93 |
| | INTRONIC | 0.93 |
| | INTRONIC | 0.86 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | 3PRIME_UTR | 0.93 |
| | 3PRIME_UTR | 1 |
| | 3PRIME_UTR | 1 |
| | 3PRIME_UTR | 1 |
| | SYNONYMOUS_CODING | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 1 |
| | INTRONIC | 0.92 |
| | INTRONIC | 1 |
| | INTRONIC | 1 |
| | INTRONIC | 1 |
| | INTRONIC | 1 |

SNP: reference sequence identification number according to dbSNP; type: location/function of SNP;
CHR: chromosome number;
MAP: physical location of SNP on genome build HG18.
tagged SNP r²: Linkage disequilibrium (LD) in the observed regions was assessed by calculating r² according to Hill et al. 1968.²⁵

It is known in the art that two or more alleles, polymorphisms or SNPs may be associated in a non-random manner. Such SNPs are also referred to as being in linkage disequilibrium and are inherited together. A genomic region comprising a group of SNPs being in linkage disequilibrium may also be referred to as haplotype block. SNPs being located within the same haplotype block may also be referred to as “tagged SNPs”. Each of the SNPs provided in Table 1 may be replaced with a corresponding tagged SNP from Table 4 for the purpose of the present invention. Corresponding tagged SNPs for a given SNP provided in column 1 of Table 4 are those tagged SNPs which are listed in column 5 of Table 4 subsequent to the given SNP. For example, SNPs rs1872133, rs1872132, until SNP rs12899914 are tagged SNPs corresponding to SNP rs1630535. Preferred tagged SNPs are those with a correlation coefficient (r2 value) of 1. The risk alleles of the tagged SNPs are those alleles which co-occur with the risk allele of the respective SNP as listed in Table 1. “Co-occurrence” in this context refers to those alleles of a group of SNPs within a haplotype block which occur together. Determining co-occurrence is within the skills of the skilled person.

The figures show:

FIG. 1 Distribution of risk alleles in cases and controls applying the subset of 18 SNPs according to the invention in the discovery sample (see Table 3).

FIG. 2 Receiver Operating Characteristics (ROC) curve of the number of risk alleles as a predictor applying the subset of 18 SNPs according to the invention in the discovery sample. The area under the curve is 1.0 hence yielding a perfect classification.

FIG. 3 Distribution of risk alleles in cases and controls applying SNPs as defined by SEQ ID NOs: 1 to 100 in the discovery sample.

FIG. 4 Receiver Operating Characteristics (ROC) curve of the number of risk alleles as a predictor applying SNPs as defined by SEQ ID NOs: 1 to 100 in the discovery sample. The area under the curve is 1.0 hence yielding a perfect classification.

The following examples illustrate the invention but should not be construed as being limiting.

### EXAMPLE 1

### Methods

Discovery Sample—Patient Recruitment

We recruited 397 patients aged 18 to 75 years who were admitted to the hospital of the Max Planck Institute of Psychiatry (MPI), Munich, Germany, for treatment of a depressive disorder presenting with a unipolar depressive episode (85.7%), bipolar disorder (12.4%, 6.1% bipolar 1 and 6.3% bipolar II), or other primary diagnosis with current depression (0.5% dysthymia, 0.7% adjustment disorder). Patients were diagnosed by psychiatrists according to the Diagnostic and Statistical Manual of Mental Disorders (DSM) IV criteria and were included in the study within the first three days after in-patient admission. Patients with depressive disorder due to a general medical or neurological condition were excluded, as were patients with a lifetime diagnosis of intravenous drug abuse and depressive symptoms secondary to alcohol or substance abuse or dependency. We recorded ethnicity using a self-report sheet for nationality, first language and ethnicity of the patient and of all four grandparents. All individuals included were Caucasian, 85.1% were of German origin. The study was approved by the local ethics committee, and written informed consent was obtained from all subjects.

The study is designed as a naturalistic pharmacogenetic study (Munich Antidepressant Response Signature (MARS) project) to reveal genetic biomarkers specific for depressive disorder, clinical outcome and severe side effects. All patients are treated with antidepressants according to doctor's choice. Concomitant psychotropic medication with mood stabilizers, neuroleptics, benzodiazepines and hypnotics is allowed. For all patients plasma concentrations of antidepressants were monitored to assure clinical effective drug levels.

### Replication Sample

The German replication sample consisted of 501 Caucasian inpatients from the psychiatric hospital of the University of Munster and from a second patient recruitment in the Max Planck Institute of Psychiatry (MPI), Munich. Gender distribution (p>0.2) and age (p>0.9) did not differ between samples. Overall, 85% of these patients suffered from major depression, while 15% were in a depressive episode of a bipolar disorder. TESI positive were N=42/8.4%, patients completely lacking suicidal ideation N=149/29.7% and N=434/86.6% without increase in suicidal ideation. Psychiatrists ascertained DSM IV diagnosis. Patients were rated weekly from admission to discharge (Munich) or until week 6 (Munster) using the 21-item HAM-D rating scale. Ethnicity was recorded using the same self-report questionnaire as in the MARS study. All patients were Caucasian and 90.7% were of German origin; the remaining patients were of European descent (Central Europe: 3.9%; Eastern Europe: 5.3%; Mediterranean: 0.1%). Same inclusion/exclusion criteria applied as in the MARS sample, and outcome under antidepressant treatment was evaluated accordingly.

Psychopathology and Phenotype Definition

Severity of depressive symptoms was assessed at admission by trained raters using the 21-item Hamilton Depression Rating Scale (HAM-D)^{26.27}. Patients fulfilling the criteria for at least a moderate to severe depressive episode (HAM-D>=14) were eligible. Ratings were performed within five days of admission and then weekly until discharge. The severity of suicidal ideation was rated by item 3 “suicide” of the HAM-D, with 0 “absent”; 1 “subject feels life is not worth living”; 2 “wishes he/she were dead or any thoughts of possible death to self”; 3 “suicidal ideas or gestures” and 4 “attempts at suicide”. TESI was defined as an increase of suicidal thoughts in patients without suicidal ideation (item 3 of the HAM-D=zero) at hospital admission (n=32). The non-TESI comparison groups were 1) all individuals without increase in suicidality independent of baseline suicidality (no increase of the HAM-D item 3 over time; n=329) and 2) a sub-group of 1), patients rating zero on item 3 throughout treatment (item 3 of the HAM-D=zero at all visits; n=79). In the STAR*D trial the suicide item (item number 12) of the Quick Inventory of Depression Symptomatology-Self Report (QIDS-SR) was used to define TESI. Both scales should identify similar patients as the QIDS-SR has been shown to correlate well with the HAM-D²⁸⁻³¹. The observation period for TESI was the first 12 weeks following in-patient admission, congruent with the observation period used in Laje et al., 2007

DNA Preparation

After enrolment in the study 40 ml of EDTA blood were drawn from each patient. DNA was extracted from fresh blood using the Puregene® whole blood DNA-extraction kit (Gentra Systems Inc; MN).

Statistical Analysis of Genetic Associations

Exact tests on Hardy-Weinberg equilibrium (HWE) were performed for all SNPs SNPs with a minor allele frequency below 2.5%, with a call rate of less than 98%, or displaying HWE deviation at an error level of below 10⁻⁵ were excluded from the analysis. Final analysis was done in 371.335 SNPs. Both allelic and genotypic association tests were applied. To avoid false positive associations due to small cell sizes we used permutation-based p-value estimates (100,000 permutations, in 4 best SNPs 100,000,000 permutations) in addition to asymptotic p-values. If not otherwise specified, permutation-based p-values are reported. Genetic association was tested using the WG-permer, a C++ based statistical program for rapid permutation (http://www.wg-permer.org) Phenotypic analyses were performed using SPSS version 16.0.

### Results

The comparisons of disease-related and sociodemographic variables between TESI positive and negative patients using both the broader (comparison group 1) and more restricted definition (comparison group 2) are listed in Table 5. Except for age at onset there were no significant differences among these groups. 59.3% of patients with TESI developed this side effect within the first 2 weeks after hospital admission. Current medication at the onset of suicidal ideation were SSRIs in 37.5%, tricyclic antidepressants in 34.3%, venlafaxine or duloxetine as dual serotonin and noradrenalin reuptake inhibitors in 25%, mirtazapine in 28% and reboxetine as a selective noradrenalin reuptake inhibitor in 12.5%. Antidepressant monotherapy was administered to 62.5% of the patients, while 37.5% received combination therapy. There were no significant differences in psychotropic medication among the three groups (Table 6).

TABLE 5
Table 5: Sociodemographic and clinical features of patients in the
discovery sample developing TESI compared with patients without
worsening of suicidal thoughts under psychopharmacological treatment
(1) and with patients completely lacking suicidal ideation (2).

| | TESI | Non-TESI (1) | Non-TESI (2) | |
|---|---|---|---|---|
| | n = 32 | n = 329 | n = 79 | χ² or |
| Characteristics of discovery sample | 8.1% | 82.6% | 19.9% | t-test |
| Male | 43.8% | 44.8% | 49.5% | NS |
| Female | 56.3% | 55.2% | 50.5% | NS |
| Age (mean +/− SD) | 47.1 (16.1) | 49.3 (14.1) | 50.8 (14.3) | NS |
| Employment status | | | | |
| employed | 62.9% | 63.4% | 61.1% | NS |
| retired | 25.7% | 24.1% | 23.4% | NS |
| unemployed | 11.4% | 12.5% | 15.5% | NS |
| Living with partner/children/ | 54.3% | 60.9% | 64.5% | NS |
| personnel | | | | |
| Single | 45.7% | 39.1% | 35.5% | NS |
| Age at onset (mean. +/− SD) | 32.6 (16.0) | 37.3 (15.4) | 40.6 (15.6) | * |
| HAM-D at inclusion (mean. +/− SD) | 24.5 (7.1) | 26.6 (6.6) | 22.8 (6.5) | NS |
| Previous episodes (mean. +/− SD) | 3.2 (4.0) | 2.8 (5.3) | 2.7 (5.5) | NS |
| Recurrent depression | 56.3% | 58.2% | 60.4% | NS |
| Psychotic features | 18.8% | 11.9% | 13.9% | NS |
| Illness duration in years | 13.9 (15.4) | 10.6 (10.5) | 10.3 (10.1) | NS |
| (mean. +/− SD) | | | | |
| Duration of current episode in weeks | 38.0 (58.4) | 39.1 (69.3) | 28.4 (21.7) | NS |
| (mean. +/− SD) | | | | |
| Family history of depression | 46.9% | 46.6% | 40.5% | NS |
| History of attempted suicide | 18.8% | 28.7% | 16.2% | NS |

* p = 0.012 TESI vs. Non-TESI (2).
NS = not significant.

TABLE 6
Table 6: Psychotropic medication at onset of TESI compared
with medication of control group in the discovery sample

| | TESI | Non-TESI (1) | Non-TESI (2) | |
|---|---|---|---|---|
| Medication | n = 32 | n = 79 | n = 329 | t-test |
| SSRI | 37.5% | 37.8% | 35.7% | NS |
| Tricyclic | 34.4% | 20.4% | 22.9% | NS |
| antidepressants | | | | |
| SNRI | 25% | 19.4% | 19.2% | NS |
| Mirtazapine | 28% | 35.7% | 34% | NS |
| NRI | 12.5% | 2% | 4% | NS |
| Neuroleptics | 21.9% | 17.3% | 22% | NS |
| Mood stabilizers | 46.9% | 35.7% | 29.9% | NS |
| Benzodiazepines | 50% | 20.4% | 30% | *# |

Medication was according to doctor's choice. A combination of two or more antidepressants was also allowed (TESI = 37.5%; Non-TESI (1) = 23.5%; Non-TESI (2) = 24.4%; no significant difference).
*TESI vs Non-TESI (1) p < 0.05;
#TESI vs Non-TESI (2) p < 0.05.
NS = not significant

Provided are 100 SNPs, which were significantly associated with TESI with an empiric p-value <0.001 comparing to both non-TESI groups (see Table 1). SNP rs1630535 showed a p value of 1.3×10⁻⁷, OR 10.535, CI 4.2-26.4 comparing to narrow non-TESI group, and p value of 2.45×10⁻⁷, OR 4.16, CI 2.3-7.4 comparing to broader non-TESI group. Fisher Product Method over all 100 SNPs revealed a significant association comparing TESI vs non-TESI(2) with allelic p=1×10⁻⁵, genotypic p=1×10⁻⁵ and combination of allelic and genotypic p=1×10⁻⁵. A discriminant analysis patients using this subset of SNPs revealed a 100% probability to classify TESI vs non-TESI cases correctly, no matter if narrow or broader non-TESI definition was applied (narrow definition, 32 vs 79: chi square=273 and df=91; broader definition, 32 vs 329: Chi square=429 and df=96). Furthermore, the distribution of the number of risk alleles present in each individual yielded two separate groups, with TESI cases having 85 and more risk alleles while non-TESI individuals having 80 and less risk allies (see FIG. 3).

100 SNPs with p<0.001 from the discovery sample were tested in a second independent German Caucasian sample (referred to as “replication sample”), revealing 16 SNPs particularly significantly associated with TESI in both discovery and replication sample.

TABLE 7
Sequences of the SNPs of SEQ ID NOs: 1 to 100; in no particular order. The polymorphic
position is indicated by square brackets, for example [A/G] in case of rs6793017

| SNP | |
|---|---|
| rs# | Sequence |
| rs6793017 | ATAATTGTTTGAGTCACAGAGGTTCCTACNACTCCATTAAGACAGAAGGTATAGCCTAGAGTTGGTGGGTCTGAGACAGTGTCTC |
| | CAAAGTCTGAAGGATAATAACTGAATATAGACTCCAAAATTTAAGGCTTCAGCATCCTCAGAGTCCTTGCATTTGTGAGAGCTAT |
| | GTCTGTGATGTTGCCGTTTGCACCAGGAT[A/G]CTTTATGTTGTCTTTTAAGTGTTAGGTGTCCAGATGCAGATCACCTGCATCT |
| | TGCCTCATATGTCTCTCTGAATGTAAAGATTTTGCCTACATCTGATGAAAACTGTCCACTGTTCACATTATTTGTCATATGTATC |
| | TGCTTGGAGCAGAGTAAGACACTTCTAAGGGAACTTGACATTTCATGACCCAGGATATCAGAT |
| rs1265235 | ACTGCATCTGCGTAGACNTGTCATCCAGTATACTCATGCGATGTCACTCATCTGCCCCCTCCTACATAAATGATACACAAAAAAG |
| | TCAACAATCAGAGAAACCCAAGCTCCTGGATGTCAACATCCACTTCTGCTTGGAGTTGGTTCCTTTCATGGAGACATACACCCCT |
| | CGCCACACACCTGCCACCTCCCTGACCCTC[A/G]CAATCCCTGGCATGAGAAGCTCCACCTTGTCAAGAGCAGACATTCTGAAAG |
| | GCTGAAGCCAGGAACCTTAAAGGAAACATTCTGCCAAGTCCACCACGTAGGATCAATGGAGATGGTGGTAGTGTGGTTATTGGCT |
| | TACCCAGCTCTGGGTGTAGAGTGCTTTGGGGAGGAGGAAGGAAGAAGAGCTGATCCTGCCTTCT |
| rs4881394 | ATCAGTGGCAGAGACTCTCCCTGCTCTCATGGGCTCTGTCTATTCCCTCTCTACATAATNAGAGTAAAATCCAAAGAGCATGAGG |
| | ACCAAATCAAATATGACAAAGACTCCTTAGAGGCGTGTCAGTACTGGTAACACCTGTTTTTCACAACTCACGGTGACTAAGGCAC |
| | AGACCACCCCACACAGTGGCACGGATTCTC[C/T]TCTTGAACAGACTTTCCCCAGGCTCCTCTGAGTCTCTTTTGAAATAGGCCT |
| | CGCCTTGGTCTGTAAGGATCTGAACACACCCAAACATAATTTCTAACAGTTCAAGGCTACATCAATAGATAACCAAGCCCTGTTA |
| | AAGAGCCAGCCTGAGAAAACTCACGGCTTCCAAAAGAATTCAGTATTTGTTCCAGCAATGTCTG |
| rs4724701 | CCCTGGTTCCACCGCAGGCGAAGTCAAGATCCAAAGCAAAGTCGAGGACACAGTGACCGTGTTTTTAAAACCGCCATGAACCAGG |
| | GCGGACCNCGACACCCAGATGAGAGCCGTGAGGACAGCACTGAGAATAATCACTTAGGGCTTTATTTACTGACACAAGAGGACTC |
| | CACGGTGTAGTGTTACATTTTTAAAAATCA[A/G]ATTACAAAACAATATGAGAACCCTATCTCCAGTTCATTCTCCAGGGAAAGT |
| | TGGCAGCATTCACTGTTGGGAAACCTTCACTTAAAAATAGGTTGATGTTGGCCAGGCGTTGTGGCTCACGCCTGTAATCCCAGCA |
| | CTTTGGGAGGCTGAGGTAAGCGGATCACCTGAGGTCAGGAGTTAGAGACCAGCCTGGGCAACAT |
| rs635546 | TCTGCCTGCCTGGGGCCACCACCCACCCAGTCATACACATAGGACAAGCTCAAGAGCCCTCCAACTCACTTAGTCACTTCCCCCA |
| | AATCAGATGTGACTGAATCCTATCGATTTTGCTTCCTAAATAGCCTGGGAACAGCCCCTCTCCACTCTGGCTGCCATTTTCCTAT |
| | GGAAATGACAGTGTTTCTCACCTAGATTAC[C/T]TTCACCACCTCCCACATAGCCTCCCTGCCTCCAGCCTTCTCCCTTCTTGGA |
| | TATGCACCAAAGTGACCTTTATCTAATACCCAGGGTTAATCACTTCCCACTCCTACTGAGAACANTTCAATGGCTCCTTATTGCC |
| | CCCCAGTTTCTCAAATGCCTTAGATCAGTGGTCACCAACCATTTTGGCACAAGGGACCAGTTTC |
| rs1884641 | ATGGATTTTAAAAGGATCTCAAATGGTATCTTCTAAAATTCTAAGCATTCTATCAATCCTCATGCTGAAATATGACTTGGTGAGT |
| | GACACTTCAAAAAGAAAATAAACATGCGTCTCAACCCATTACTACTTTATTTCCTCCAGATCATTTCCTCACAGGCAGGGCAGCC |
| | ACGTCAGTGAACCCACTCAGATATCTTGGT[G/T]TCTACCACTTGGAAGCCTCTTTTCTACATTCCATTTTGGGAGAGAGCTGGC |
| | TCTTCCCGGAGACTTGACAACCTGGTACCGTCTCCTCCCACAATCACCCTCCCATTCTTCTGGCTTATCTCTGGTGTCAGAGAAG |
| | AATGTAATCTTTAGGCGTGTACATTCTGACTCCCATGTTTTGGGGAAGACCCCTCAGCGTAGCT |
| rs9302822 | AATACCTAAGTAGGGTGATTGGAGGTCAAGGACCCTTGTAGGAATGAAGTGGATTTGCCTGGATGTAAAACTACATCTAAGATGT |
| | TATGAGTGAGGAAGAGCCCAAAACTCAGAAATCTGTTTGAAAAACTTTGTTAGAAAGCANACATACACATGCAATTGTTTTACAT |
| | AGAGGGCAATACTGCAAAAGATAATGAAGG[A/G]ACTATAAACAGTGATTATCTTTGTTGTAGCTGTGCAGTATGACTAAGAGAG |
| | GGGCAGGGAAGAGATGAAGCTTTTGCTTTTAAGATTNCACCTTTATTCTTTGTGATTTTCAGCAAAGATGCCCTATTTGGATAAT |
| | TAAAAAATANTTAAGAGAAAAGCCTCCATTTTGCAAACAACCTGTGATCACTTTACCCCTGAGC |
| rs3801033 | ACCTGACTACTGTACTTGCTATCATGCACATTAAGTGAAACGATATCAACCGTAACCCCTATAACCTGTGCTTTCCCTGAACCCA |
| | CTGCCAAAATATCAACAGATCAAATGAGAAAAAAATCTGAGAGACAATACTTTTCACCACCTGGGATCACTATATGCNGCCAAGC |
| | TAGGATGACAGTCATCTAACAAAAATATTC[C/T]GTTGCAGGAAAAGCAAGCTATTTGTTGCACAAGGAGGTTCCTAAGATAAGT |
| | CCAAGTAGAGAGTTGTTCTGGAGGAAGTGGGTCTCCACTTTACACCTTCTCATGTCACATCAAGCTGGGAGAGATAGGTCATGAT |
| | CTATCTCTTATCCTTGCCTTANGCTACGATCACTGTGTGCCTGATCAATGCCCAGCACATTTCA |
| rs2662090 | CTTCAGGTGGTTGAAGCTTAAATGAGGCGGACAGAGATGAGCTACAGAGCCCCAGGCTGTAATTTGCGCTAACCAGTGGATGACC |
| | TNCCAAGGCATGAGTTAGGCATTTGGGATTACATCTTCAACTTCTTGGAGCAGTGAGACACGGTGGACAGAATATCAGCTTACCC |
| | CTCAGCTCTGCTTTTTTGGGGAATTCTTGG[A/C]CAGGAGATAAAATGAGAGGACATTATTGCTGGAAGCATCTATAGAGAGCCT |
| | CTAGGACAGTAGTTACCAAACTGAAGTGTGCATTTGAACCATCTGGGAAGCTTGTTAAAAATGCAGGTTCTCAGCCAGGCGCAGT |
| | GGCTCATGCCTGTAATCCCAACACTTTGGGAACCGAGGTGTGCAGATTGCTTGAGCTCGTGAGT |
| rs279553 | TAGCTAATGTTGTTGCGGGGAAAGGAGAGAAGGCCCAAGAGAGAGGGCAAAANGCTCAGCAAATACGGGTATTCCCACTGATAGG |
| | GCATGGCCACCTGATCATGTGACAAGAGCCTCAGGTGTCCCACGCTCATCTTAGCAACCAGCAGCAGCCATATGACCAGATGTAC |
| | GTAGATCAGCTTCTTGATTTCATACTTGAG[A/G]GTCACACTGTGGGGCAGAGCAGAGGGAGAAGTATCTTTCAAATTTTTCTTA |
| | CCACCAACTATATGCCAGACAACTGTACTGGACACTGGGATACAAATTTGAATAAAACAGCCTAGAAGCCGGACACAAAGAACAG |
| | AGGAGGAAAAAGTAATTAGAAAATGAAGTGAAAAAGTTCTNGTAAATATATGTTCCCAGATATA |
| rs279542 | GTGTGTTGCTTGAAAGGTGTGGCCTTAGACACTAGGAGATGATGGGTAGAAGCTCCTTGAGAAACCTAGCAGTGGCCATAGAGAA |
| | GTAGACTGGGTATGGGGAACCTAAGTCATAGTTGTAACCTACAGCTGATTGGTAAGAACTATGGGCCAGACATTTAATCTCTCCA |
| | TCTATGAAATAGTCCATTCTCATTTATCTA[C/G]CTTAGGTTATGGGCTCTTCTTTTAGTTGGATATTCACATTTATCTTTCAGT |
| | GTTAACTGGGTATCAGTTATGAGTCTACTACTGTATAATGGCATCCAAAAGAATTTNAAAGTACCTCNGTTTATAGGGATTTACA |
| | ATTCAGTAGAAACTTGTTATTCATGTGAAACAGTGAACATAATATCTAATGTGTGGAGCAAGT |
| rs11250017 | TCTTATAATAAATCTATCTGTCTATATCTATATCTACATCTGTCCTCCTACTGGTTCTGTTTCTCTGGACAACCCTAAGACTAGT |
| | ATATAAAAAATAGANCAAACTTTCAGATTGAGAAAAAAGACATACAAGGCAAATGCATAAGAGATAAGTTTTATGATATATTGAT |
| | ATTCAACATCTTAACATAATTAAATGAAGA[C/T]GGGAAGTCATTGAAAGAACAAAGGAGAGTTCATGTTTACTGCTACAATGTG |
| | CAATGAAGATATAAGTTATTAATATTTTTGGAAATTTTAAAACATCTCTCCCATANCTATACAAATCAAGTGGACCAAAAAATTG |
| | AGTAAGGATGTGGAAAAACCAGCATTGTCAGTAAGCTAGAACACATAAATATATATTGAAGTTG |
| rs1013120 | CAAACTTAAAAATAACAATTTTCCCAGAAGGCCAAATGTTTAGGGAACATTTTCCCCCAGAGGACTCAGGGGTAAAACGAATCTG |
| | AAGACCTTAATTATTCAATGTCCCCAAGCCTCTCTTCCTAAACGTGGAATGNGGATGAGAAGGAATCACTTACAAGAGAGAGAAC |
| | TGTGTGCTAAATATCAGGAGACAAGTCGCC[A/C]AAAATTCTAGGTAATGGTAGTAAAGTAAGAATAGGCANCCTAAGGTATCAA |
| | AGGTCAAACGTGGTCCCCCAAACACCTGACCTGCAAACCTAGGCAGATAGCAGCTCTAGAGAAGAAGAAATGGCTGGAGCAGAGA |
| | GTACGTGGCTCAGTATATTAGACCTTATTTGCCACATAGATGACTTCCAAAAATACTCTTTTGG |
| rs11258903 | AAAATTTTAAATTAAATCCAAAAATACATAAAATTAACAACGTTCAAATTTATTGCTTNACCCTGATATTNATATTTTTTGGCTG |
| | AAATGAAATGCAGTTTTTAGCATGAATATGTTTCTGACTCCTCTTCTGTGGGTTCTTTTGAGATTGGCATGTCTATATTGCCCCG |
| | TGCTGGGTTAGGAGTCAATTCTCCTATNG[C/T]TTTTTTCCTACCCAACCTTCTGCAAACCTTGAACAATACAGCGAGAGGCCCT |
| | TAGCTTACATTTGGCAGAACCACATCATTTGTTACTAAGTAAATCTCTAGTCTTTCTTTGTTGGTCTGAAAGAAATATGGCAGGC |
| | AAAACAATCCTGGGCCAGTACTCAACTATGAAGTGGTCATCAGGTGGTCCCCAATCCTCTTAT |
| rs7645289 | AAGTCACTTGCTTTTTGTTTTTTTTTCAGTCTCCAGATTGAATAGGCAGACTGCCCAAATCTCCCTTTCTTTCATTTTTTCACTC |
| | TCTCTGTCAGAAGAAGGGGGCATTATAGGACAGTATTATTAGGCTAGATCTTTGCCTTGTTTTAGTGTCTTATCCACTGGCCCCT |
| | TCATAGGGTTGGGAAATGGTTTCATAAGAT[A/G]GTAGAAAGGGTGGCATAGGTACTTGCAGTATGGCTAACAGTTGTTCACCAT |
| | GTAGCTTTAGATATCTTAGACCCAGTTCCCAGCTGTGCTGCCTTAGTCACTCTGGTCCTCGTCTTCCTCAGTATCTCCTCTATGA |
| | GGTNGGCATCCATGCAATTCACTCCAATTGCTTGTACATTCTTACATTAAGTTTTGGGGGATC |
| rs6035712 | TGCCCGGCCTGTTCATCTGTTTTCACACAACTAAAGTTGCTCAGTTGTTGTGCTGGAAAGGACTTCCCTGCCCAAAATATACCAG |
| | CAAGACATCATGCAACCGGAGCAGGCTGTGCCACAGACCCTGGCATGCAGAGGTGTTGTGTAAACACAGAAGGGATCTGGGCAAA |
| | TGCATGCCTTCATTTATAACAGATAAAAA[G/T]AAAGAAGTGTGCCAGACAGTGAGGCACTGACGCTTCACAGGAGGGTTCCGAG |
| | AGCTTTCCTCCCTGCCTGAAGTTCCTTCTCAACCCAAGTAGCCACCTTGTCCTCGTGTTTGTAGTTCCCCTCTCAATCCAAGCAC |
| | TATCCTTGGCCTGGGGGGGTCATCATCACCACCAGCAAGGGTAGCACTGCAGNGTGACAATC |
| rs7307064 | TTGAGAAGAAAAAGAGCAGTTCTTTTGGGGGAATTTAGTTTTGGTCATGCTAATTTAGATACACATTTCTGATAAACAATTAGGA |
| | GTATAANTTTAANGTTTAGCNGTTAGCTAATTAGATATGGGAATAATGTTTTACACTTTGGCAGCAGATCCACAGAGACAACATA |
| | AGTCATGGTAATACTTAAGCAAGGGTGATA[A/C]TCATAAGGGATCAAACCATTTAGGTTGAAAATAGGGCAAGAGTCACAAATC |
| | AGGTAACTTCAGTGGCCACCAGGTAACAACTATGTGGCGCTATCTGGTTAAGAAAAGAGGGGCCCTTAATAGAAAGGCGTATACA |
| | TCCCGTTTAAGGCATTTAAATCNATTTAAAATATATAAATAAATAAAAACAATGCCCATAATAA |
| rs2270101 | ACNAAGGGGCAGACACTGAGACTTCAGCAAGTTAGGAGTTTGTCAGTAAATTGGAACCAGAGTTGTTTCTTTCTGGGCTATGCAG |
| | TTGCTAGATTGCTCTCTGCTTTAGCCTTGCTGTCTAATTGTCATATGTAGGAATCATGCAACTGCAGAACTGTGTACCCTGTCTC |
| | TTAAAGCAGCTCTGTAGTGGTCATTCTGAG[C/T]GAATTAATTTAGGATTAGCTCAGAAATAACTAATGCCTCCNGGGTAGCCAG |
| | AATTGCAGTCTGCCTATTAACATTTGCTTTGTTCCATTGCTTTAGAGTCATATTCAATTTATCAATTTCCCTCTTCCAGTTTTTG |
| | GCATGGTATCCTTTTGTCCCTTTCTCCTGTATCATAACTCTCAGGGAGCCTAGCTTGCTATGTG |
| rs7788668 | CTTTTTTTTTTTTTTTAAGAGATAAGATCTCGTTATATTGCCCAGGCTGGTCTCAAATTCCTGGGCTCGAGTGAACCTCCTGAGT |
| | AGTTGGGACTACTACTCACCACACATGGCAATAAAGATTCTAAAGTTAGAAGGACAAAGTGATGTTACCTAAATTTGCCTGTGCT |
| | TCTGATATGACAAATAATCCGCCACTGGTG[A/C]ATGGCCCATAGAGCTCATCTTCATCTTTGGATGAATACGAGTGGACCTGTC |
| | ATACTCCTCAGCCCTATGACATGTAAAGCACAAAGCCAGGAATGAGGTCATTGTOTTATCACTTACCCCCAAAAGCTCTCAATGG |
| | CTCAACTTACCTAAGTCTTCCTCCTTTATTCTCTTCCTTCATATCAAAACTTCTGCAAATTTTT |
| rs1891877 | ACCCAGAGCCAAGGGTAAAAGGTCTGGGCTTGTTATAATTTATCACCCATCTCAAATAATTGGCTATCATCAAGAATTAGATTCA |
| | TGCAAGAATCACTTAAAGATTCATTTGTCTGTCACATTAAAAACACTAACCTATCTTTTTCTTCTTCCTTAGCAGATTTTTCTTC |
| | GAGGAGCACAAGGGTGGGTCGGTGGAAC[C/T]CTTCAGGAGCTAGGGTGTCGGAGGTGGGGGACTAGCCCTGGTCCCACTGTCCT |
| | TCCTCCCCTCCTCACTCCCCAAGCTGCCCCTACCCCCTCTCTTCTCCTTGGGAGCAATCCCCCATCTCCATGCTTGTGACACACA |
| | CTGAGCTTACTTCTGAGCACTCAGTTAATAAAGGCCTGTACATTGAAAAATATTTTTAAAA |
| rs7350731 | TCAGCCTTTCTAACCTCACAACTTACTTCCAGACCACAGCATTAATCCATGCCCTTTATGCTCCAGTAAAATATTTTAAAGTCCC |
| | AAAAAGTCTTCTCTGACCCCTGTNTCCCCCAGGATTTGATATGTATCCTTTGCACCTCTTGTGATATTTACCTGAACATAGCACT |
| | TATTGAAATTGTTTAAAGTTTTTCTACTCA[A/G]TGAACTGTAAACTCATTAAGAGAAGGAACGATGTGCTTTTGACTGTTGTTT |
| | CCCAAGCACCTCTCAAGGCAGTTGGCACATTAACGTGATCAACCAATATTAGTTGAGTAAATGAAATGAGTCCCGGGGGACTCAC |
| | ACTAGCTTGAGGGTCATGGGCCAATTCCAAACTCACTGTCTGTAGGGGGATAATGAGTCTGATG |
| rs4795069 | CTGGGAGCTATTTAAAAATGTAGAATCTCGGGCCCCATCCTAGACCTTCTGAATCAGAATTTTAGCAATATTTCCATGTGCTTCA |
| | TATGCACATTATATGTTACAGTTTGAGAGGCATTGTTCTAGAAGGAGCTCAAAGACTCAGGGATGTGAGAATATTGAAATGGAGC |
| | TACTATGTGCAACACGCTTCACACTACCC[A/C]CCTAGCCAGAATCCCCGAGCAAGCCCAGGGGATGCACCTATCACCAAGGAAT |
| | TAAGACATGCATTGGAGAGGAAGGTACTAGCGTGCTGGAGTGAGGGGGGAGGCAAGAAGACACNCAGGATACAACATTTAACCNG |
| | GCACCCACCCTCCGGTACTGATCCTGAATGCATGAGCCTGAAAGTGAATGCCTCCTTTAATA |
| rs4544214 | TAAAAATGCATTAACGTCTTTTTATATCCATCAAGGGAAGGATGAAATGTTGAATTTGAAGACTAATTCAGTAAGAAGTCCTAGG |
| | GGTTTAACTGTACATACTACCTGAACTGGCTTTTCTGAGAGATGAATCAATAATGAAACATGTCTGTTTTAAAAACTACCACATG |
| | TGACTCCTATTTTTGTTAGCTGAAAGCTGC[A/T]ATACGGAGTATTACAGNAATGTGAAGGTGACTAGCTTGAAGGTAGGGTAAC |
| | TAGAGAGCCAGAAAAGTTTTGTTTTAAACTTGATTTAATGCGTTTTTATTTTTTCTTATACAAAATAGAGATAATGTTGCTAACT |
| | TCATGGAATATTTGAGGAAATGATATGAAAGTGTCTGGACGTGCAGTAACCTCATGGNTTCTTC |
| rs584762 | GATGTTATTGAAAAAGATTGGGAAGATTATGGGTAGTAATAGAGATAATTGCAAAGGAGAAACAAGAAAAAAAATTAATGAATCT |
| | TCCATCTCAGGGAGAAATAAATGGCTTTATTATGGGGTCAGGAGGGATGATCCTAAAATTCCCTGTCCAAGAGAAAATGCATAGT |
| | GGTAGTTTGGGAGTTGGAGAATTTGGCANC[A/C]ACAACATCCATGAATCAAAACAGCTTTTAAGGGACAACCTCAGGGGATTGC |
| | ATTTCTATAAAAGGAATGAGATGTTTTTCTTTATCTGCTGAAAATGCAGAATCAGAGCCGACTAGAAGCACAGCTAGCTCCTTTG |
| | TCATTATATGCAAAGGACAAGAAGAAACATGATGTTCCACTATAATAGGCCATAGGCAAAAA |
| rs761453 | CAAGTAAGTAAGGATGTGTATAAAGACCCTAGCCCATAAATGGTCATCACTAAATAGGTAGTCATTATTTATCATTGGNATCTTT |
| | ATTTGTGAGCTGCAATGGATATGTAAATAAATATAGGTATGTATGCATGATGCACAGGTATCCACCTGTGTATAGCAGTTGCACT |
| | ATTTCCCTTGCCTAAGGAGAAACTGAAATG[A/G]TAGTTTAGGCCAAGAGATGATTCAGACCCATGTGTATGACTAATTACAGTA |
| | CTTAACAAGAAATTGTAATAATATCCATTGGTATAGCTAGAGGTATGAGTTTCTGCTAACTCAGGATCTAGTGTCACCTTCAACA |
| | AACTCCCCTTATTCTGAATGTATCTAGAAGCTCTAAATGCTTTGCTGGAAACTGAAAGGGTAGA |
| rs12583395 | AAAAAATAAGATGATTGGAGGACAATCAGTAGTGTCTGTGACATCTACAACGGAGCAGTTGGAGAGGAGTAGGCAAATCCCTATC |
| | TACTGGCAAGAAAGATGTCCAAGAACATTTCCAAGTGAGGACGCAGGTTGTGAGGTCATATGTCCATTATCACCCATCTAAGGTT |
| | CTGAAAACANACATTTATCAAGGCATGGAG[A/G]AAGTTCTGGAAGTTACACTCTTTTTCTCTCAGAGAAAGCTGGAATTGGGGT |
| | TTGGAAAGAAGAGGAAATATGTCTATTTACTCAAATACTTCTGAATTCTTTAATTGTTGAAATCCTCTAAGAACTTGTATTCTGC |
| | GGTCATTTTTAAATACAAGGGAAAAACAAACAAACAACCCAGGCTGTAAGGAAGAAAGTGATA |
| rs2070439 | TCAGCTGCTTGGGAGGCTGACATGGGAGGATCCCTTGAGCCCAGGAGTTTGAGGTTAGAGTGAGCTGTGGTCGTGCCTTTGCATT |
| | CCAGCCTGGGCAACAACGAGAGACCCTGTTTCAAAACCAAAAGGGAGAATGGATTACCACAGGGGCTCGACATGATCAAGTCAGT |
| | TTAGTAGGAACGACATGTGAGGCATGGCCC[G/T]GTCACAGGCATGAAGGTTCAGAATATAGCCTGGAGTACACATGTATCCCTC |
| | TGGGCCAGGCAGAGGCTTCACATCTTGGTGAAAGGCCTGGTCGCACTTTCTCTGGGTTTCAGATGGACTGTTAATGCAAAGTTGT |
| | TTTTGTGTTTTTGCAAATCCCAGGAGCTGGTGGATGGGGATGGTGTGACCCTGTTGGAACCTGC |
| rs8095186 | ACTTAATACTGGGGGCAAGANGGTTCTTGGCTGGGCACACAATGCTATGGGAACTTAAAAACAAATGGACTAGCTGCAGGTTTAA |
| | ATAATGCTACAGTCCTTAGACACTGTATATTATTCATAGCTCGTTGAACTCTATTATCTCATGTGTTCCTCTCAGTGGCTCTGTG |
| | AATTACCCAGGACAGGAAAGAAATGATAGA[A/G]TGTGAAGATAGAAGAGTCAGTAGAGGTCAGAACCCTTCCCCAGCCCCTCAG |
| | CTTTTTTTTTTTTTTTTTTTTTTTTTGANATGAAGTCTGGCTATGTCACTCTGAAGTGTCGTGGTGCGATCTGGGCTCACTGCAA |
| | CCTCCGCCCCCTGGGTTACAGCGATTCTCCTGCCTCAGCCTCCCGANTAGCTGGGACTACAGGT |
| rs583338 | TACTTTCCTATCATCATCTACCTTTGGGATATTATGTCTTTCTTAGTAAAGTGTCGCTACAAGACTTTTGTCCATTTTTTATTGA |
| | AATGTCTGTCTTTTAAACATTGATTTTTCTTTAAATATTTTGGATACAATTCCTATGTCAGGATTATACATTGCANATATACTCT |
| | CTAACTTCTACCCTGTGCCTTGCCTTTTTT[A/G]TTCTCAAGTCATCTGTAATTTGCATTCTTCCGTCTTTTTTCCCGTCAATGT |
| | TATCAGGAATTGTATACCTGGAGAGTATAAACCATATACTCTTAAGCACATTTAGAGCTACTTTCTGTAGATCTTGATATGACAT |
| | GTTATCATTATCATTCAGTTCACAATATTTTCCTCTAATTTCTTTTTTTAAATTTTATTTTA |
| rs2228246 | TTTGCCGAGTGTCCCTTCCTGAGTTCCAGCAGTTCCTTCTTGACTACCAGGGGGTATGGNTGGGCTGACATTGGCCCAGGCTGGT |
| | AGGTTGTGGGGGGCTGGGCTCATCCCTGACTGGAGGCTTCTCTCATCCCCTGCCNTCCCTACCCCATCAGGAGCTGTGGGCTGTT |
| | GATCGCCTCCAGGTGCAGGAGTTCATGCTC[A/G]GCTTCCTCCGAGACCCCTTACGAGAGATCGAGGAGCCATACTTCTTCCTGG |
| | ATGAGGTGAGCCCGATGTTTCACCCATTTTTTGTCAAGAGAATGAGTAGGGGTGACCAGGACCCCACCCGGGCTCCAGGAGCTAG |
| | ACGCTCCTTAGGGATGCCACCTTGTTTCTACCTACTNTGCACCTTGCCCACCCCCAGTTGGGAC |
| rs2664537 | CTGTGTCTATGAATATGACTATGAACTCTGAACTATTTTATCCATTGGAAGGTAAAGGAAAGGTCCTACATTGTGGGAGGAAGAA |
| | CTGATGGAACCCCATCTTGCTTGCTGCTTGCTTGGTGGTGGGCAGGCCGAGGGTAGCGGCAGGCTCTGGGCTGTCTGCGAGGATT |
| | CTGGAAGCTCTCCCAGGTGCCCAGCAGCC[A/G]GGCANGGGAACGGCATGTGGCAGCAGAGAGTCGGGTTTGGCTCTTCCACGTG |
| | GCAGGCGGTTTCCCAGACTGGCCAGTCTTCACATTAATCAGATCAAATTCAGTCTGTTTCTGAGAAGAAAACACATGCCTGTCAC |
| | TCTACGGCAGCTGCCACCACCTGCCCCCCAGGCAGCCTGGTCCTTGCTATACCAGGGTGGCA |
| rs6072317 | AAAGTAATCTTCAGTTCTTCCTCACCCATTACTTATATATACAATATATTATTATAAAGGAGACAACACAGAATATAGAAGGCAG |
| | ATTTCTAAGTCCCCTATTAGAGGTTTATAATTCTTTTATAAAATGTTATCTTTTCTCTTCCTCAACACAACAAACGAACCCCATT |
| | AAAGTCAAAACTGANAACTCTGGCCAGGA[G/T]TCTGTTATATGTAAGATGCCTCCTTGTTGATGTTGCTATAGTCCTAATTAAC |
| | AGTCGACAGGACATTTCAAGACTTCTCACTCTTATCACCAGTCCTGGAAAAGTTGTGTTTTTCTATAACTGAGGACATGGAATCC |
| | CATCTCTAGGGCCACACAGGATAAACCAAAGCCAACCTTGACCTTAAGGAAATTTGTAAGGAC |
| rs607234 | CAACCAACTGGAGAGGTTTTTAAACTCCCAGTATCCGGGGCATCAGTATTCGTCTAGGGCACAGTGATTCTAACACACAGTCAAN |
| | GTTGAGAACCACTTATTTAATTCATCCTGTAAATCCTCACAATAGAATCCTGCAAGGTAGCTTTTATCACCACCTTTGCCGATGA |
| | AATTGANGCTCAAAGAGGTAAATTGATTTC[A/G]GCATCTGTAGCCCCAACCTGTGACCTCTCCTTCCTTCCTTCTCGGGCTCCC |
| | TTCAGGGATTTCCTGATGTTACAGAAAATGGTATTATGACGCTGCCCTCCTGGCCACCTTGACATAGCTCTGCTCCCAACAGGGC |
| | TCTTCTGAGATAGCAGCCAGACACTACCTGGGATCAGGACTGATACTAAATGACTTCTGCCAA |
| rs4518023 | TGTGAGGCTAAAGGCTCCCACGCAGCATGGGGCATGGGGAGCCTGAATGTAAGCCCCAGGGCAGGAAGTTTTGCCTATTTGCTCA |
| | CCGTGTAATCTCCAGCATCTAGAATAGCATANTAGGCCTTTGGTAAATATCTGTTGAATGGAAGGACTAAAATTAGTTCTGGAAG |
| | TGTCTGGGGATGGGGCTTAAGTTACCTCTG[A/C]AGGGTCTCCAAGGCTAAATCCTTTTCCTGAGTTAGAAAAACAGGGCTGTTC |
| | TCACTGAAGTTTTGGCCTTCAGAGTGTGATGAGGAGTGCANAGGGGCACGGGGCAGCTTCAAGGACAGGGCTGGGACCACAGAGG |
| | GTGAGAAAAGGGCACAAGGGAGGAGGGGTGCTCCCAAGATGGCAGCCCCTAGAGATCTGGGCTG |
| rs7146332 | TTGGTTCTTTCCCTACCCAGCAGCCCTATACCTTGCCTTNATGCTATGTATAGCTGGAGTTTATCTCATACCATGAATAGCCAAG |
| | CATCACATGGAATATATCCTAAGAAATATTCCATAAAAATGAACTTGATGGGATCATGACAGAGATGGCAGATCTACAAATTTTA |
| | AGAATTGGAGACTATCTCATAGACTTTGTT[A/G]AGATCACAATCAGTTCTCAACTTCAATGTGGTACATTTACAAGCATTGCAA |
| | ACAGTGTGAGATCCCAGCACAGATGTCAAACCAGAAATTATGTGACTTGAATGGCTTGGAATAATGGCCCATTTGGGGCATCCAG |
| | CACTTCCACTGTTGGGTAAGTACTGTTGTCAACAGTCACACTGTCCAAGTGGATGATGCACCCT |
| rs2088138 | GTGTCACAGACAGCCCCCACCCCAAGCTCCCTTCCAGGTATCCCCATAAATCATTTGGGCACACTCCCTCTTGGAATTGCAATTT |
| | CCATCTTCCACTTATCCCTAAGGAGCTGGCCCTGTGGAGAGGTGTGTTGGTTGTTTTGTTTTTGCCAAAGGCCCTCGCTCTAGGT |
| | GCTCCTGTAAAGGTACCTTGGCCCCTGATA[C/T]GGGATGAATGGATGAACGAAGCCCCAGATGCTCCAGTGCCAAGGAGGTCAG |
| | ACCGGGAACAGGCCCCCACTCCCACACCCTGGGATCGGAGCTTGCTTGGTTCTTGCCATCTTGCAGGATGACTTCGAGTCTGGAG |
| | GGAGATACACAGTGCCTCTCCATCCCTGGGGTGGGGGGAAGGGATTCTGCTGGGACTCTTGAGT |
| rs4810622 | TAAAAACAAATCAAGAGTTGGGCCAGCTGCATGGAAGGACCCAAGAGGATTATTTAAATGGCATGCCAAATTTCACCCATGCCAC |
| | CCAATGTCCTCACCAAACAGGCAACCCAACGGGAGCTGTTGTCCTCTGGATAGCCCCAGATGGGTCCCTAACTCTAGCTGCTTTC |
| | TCACCTGAAGATGCAGAGCACCCACTGACA[C/T]TCAGAGGAGCTTGCTCTCCAGAGGCCCAGCCATCCCCCACTTCTTTACAAA |
| | TGCCCTCCTAGCTTTACTTACTTATTCAACAAACATTCCAGAGCATCTACCANGTGCACAGACCAAGTACCATAGAGAAGGCAAA |
| | TCCTCATAAAACGGCCTCCCTGCCCTCAAGGAGCTTCCAGTCTAGATTGGGAGGGACACCCCCA |
| rs2839178 | CACCCTATAAATATCAGTTGACATACAGAGTAGCCACCACGTCCCAATCTAACTCCTGACTTTTCCCCTTATATTGTATGTGTGT |
| | GTGTGTGTGTGTGTAATCAAGTATTTCTGAAATGCAGTATAATCCAATTAGTCTATTTTAGGTATTCATTGCTATAACATTTTCC |
| | CTCATCCTTTTACCACTGAATCAGAAACT[A/G]ATAGCTCATATATTTTGAATTTGTTAAAAGACTCTTCCCCGTGTGTGTGGAT |
| | TTTGCCTGTAGGAACACAGGACTCACCGCTGTAGATACTTGCAGAAGCACTGAAGCTCCTGGAGGGTCTCCTTTNCAGTCTGGAA |
| | GATTTCCTCCACGAGAAACAAGTCCACTAAGTGGGCACAGACATCCTCACAGCAACGGGCCAC |
| rs12589623 | AATCGTAGCAACAAAACAAAGGCAATTCCAAATTCAAAAAAATCATTTTTAAATTCTGCGTCTCATCATGCATTCTGATTATGTA |
| | TTAGTAAAAATATGAAATCCCACCTGGGCTTAATGCTAACTAAAATTTTAAATATTATCACTACTTTACATTTTTTAAAAAGAAT |
| | AAAACATTTTAAAAAGCATTATGTAAGAAA[A/C]GCATGGAGTCAGATTTGAGTTGGGAACTGTTATTATGCTGGTTCTTTAGGC |
| | TGTTGTTTTCTAAGGGAACTAAANAACTCAATTTTCTTATCCTCTTTGGGTGAGTGGTGCTGTAAGTTTGAAGACTAATGTAAGG |
| | CTCTAGAGTCAGAAAGTATTTTATTCCATACCTAATTTTTCCACTATGTTTTGTTATTCATTTT |
| rs2414660 | TAGTTTTATTTTCAAAACAGGAACATCCGGCAGAATCTCAGCTTTCATTTTGAATTCAGNGTTCCCGCTTCATCCATTATTCATA |
| | CACACTGCACTTTCCTGGTTTGTGATTTTACTAAATTTCTTCATTTGAATTATGGCCTTGTGGTCAACAGCGGGCAATATATTAT |
| | TCTTATATAATTTTTAAATACAAAGGCAAA[C/T]GGCACGGAACAATTAGAATCGATGGCTTGGGAGTCTGGCACTCAGATGTCA |
| | TAGGCAAATGCTGTTTTCTTGACTTTAACCTATGGGTTCCACTTCATGTGGGTTGTATTTTTCATACATCAGGAGTTTTTATAAA |
| | TTTCCTATTCAATCCAGCAGCCCTACTTTGTTCGCAAAATATAAGCAAAACAAATATGTCAGGG |
| rs1630535 | TGACACTGAAAAACCCACTCAGTCACAGGGTTAAGAAACTAATAAAATTACTATTTTTCAAGTCCTTAGTACATATTTAGCATGA |
| | TTCTAGGCATGGCACNGGCAGAAGAAAAGTATAAAATATNGCTTCTACATATAACTTACNGTTTTACTAAGGAGATGATTGGGTA |
| | AAGACAGTCCATTCAAATGCCATAATCAAC[A/G]GTGCATAGTCAAGGGCCAGCCACCAAGCAGCTGGGAGAGAGAACATTGAGC |
| | TAATATTTGGAATAAACCAGGTTTGCCAGTCAATTATCAGAAGTACCTGGTTGGTAAGGAGTCCTGCAGAGAAGAGAAGTTGTTT |
| | TCTGACAGCCTTAAGCAGTTGATCATGCCTAAAGACAACATTATCAATGTCCACTAGGCTCTTT |
| rs6868846 | AAAGAAATATGGATTGGCTATCAAGGGACTTCCACCACTAATCAATATTGGACAAAGATCATCTCACTTCTCTGTGTGTCTATGT |
| | CTTCACTTTTAAAGTGAGAGAGACAGGCTGAGTGTTTATACTAGAAAAAGCTTTAATTTCAAGGAAATCTGACTAGAATCCCAAC |
| | ATGGAAAAACAGATGAAAACCGGTTCATTC[A/G]TTGAAGCAGAGATGGGGGCCGGAAGGAACGTAAGGATGAAATGGAGGGATG |
| | GGGGAAATGGAGAGGTCAGTGAAGGTNTGACAAGTAGAAGCAGCAGTGGCAGTGACATAGTTAAACTGGGAAACAAACAGGATGT |
| | GGCAACTGGAAGGCAGGCTGTCTGAACTAGGGATTTTGGAAGCAGACCGGTGTTGCATCTTAAG |
| rs7901463 | TTAATTTATCTCTCATTTGTGAACACTGTAATCATTTCTCTATCCCACATTTATTTAGCTCTCAAAGCTATGCTATTTTATTGTA |
| | CAATGACTAAACTACTCAACATGAGAGATTTGGGAGGATCTTTGTGGTCAGCTTATTATTCTTTTCATTACCACAACAACAGCAC |
| | TACAAACTCTTTTTCTAAGTGTTTTCCAT[C/T]TATGATCTCACTTTTGTGGCTAGACCTCACCTCTAGCCCCTGGGAATTTCTT |
| | CTGTGAACGCAGCAGGATGACATCCCTCAAGTCAAATTCAAAGCCTTAAAGTAGATCAGCAGATCAGGGCCCCCTAATGACTGAT |
| | TACACACAGCTCTGACCCCACACTTAGCCCCGAGCTTGTTGTTTTCTGCAGCCTTTTATCCC |
| rs356999 | AGGCCTAANGCCTGACTGACAAGCAGCTGTTTGAAGGACACTGTCCATCCAAACATGGATTGTACNGTGGACATACTGTCTTCTC |
| | TGGAGTTACAGAGGGAGTTGGGTTCATTGCCACCCTCATTAGGTCACAGAAATCCCAATAAGATAAAATGGAAGATGCTACCCTG |
| | CCTGAAAGTGCCCAGCATTGGTGCAGGATGC[C/T]GATGGGTACTCAATAAATGGACTGCAGGTTGACTTAGTCCCCCTTCCCAA |
| | GTGGGCAGTTTAGATTCTAAGTACTTTCCAGTTGGCTGAGCAGTTTTTCCCAAGAAGTAGGCTCCTTACTGTTCCTGATTTCTTT |
| | TGTCTATCCCATCAGAAGCAGGACAATCAAGGCAGGTCTAAGAGAGAACTCATGGCAGGAGCAGA |
| rs17175096 | TTGTAACAGAAATAAAATGAATGTCATTGTTACATATTTTTTTGGTCCTTGTTAGTTCAGCAAACTTCTCGTTTCCTCCTCCTGC |
| | TATGCTGTGAGATGCATATGATGTCTATGGAAGAGCGGGAATAAAATGGTTAAATTACACCAACCCCACTCACCTCTGCTCTTTC |
| | CACTGACAGTGAACGAAACCATAGTAACCC[C/T]ATAAGAAGGCATCTGTGAAGAAAGCTTTCCTGTAAATACACCCAGTAAGTT |
| | AAGAATCTGTGTTACAAATGCAGAGACACAGAATTCTCTGAAATCACTAAAAAGAAACCTGACCAAAAATGAATGAATGAATGAA |
| | TGAGCCAACAACAAACAAAGTGAAGGGAGCTGACAGTCCTAGTACCTTGCAGTTTGTGACAACT |
| rs4939517 | TGCTCTCCAGCCTGGGGGACAGAGTGAGACTCTGTCTCAAAAAAAAAAAAAAAAAAGAAAAGAAGGTCCAAGGGCTGCNGGGAAA |
| | AAGCTTCCATGAAGCTGGCAGGAGAAAATCATGCCACCAGCCTCCTAGAAGATTTGTGACTCAACTACTTAACTCAGAGATTTGC |
| | TACCCAGCCTCAGAACTGCCTCCTCAATTC[C/T]TCCAAGGGCAGAGGACGTTTCAGATCACCAGATATTCACCTAAAACACTCT |
| | CCATCCCTCAGTGAAATGTAAGAGATCTTGATCAGCAGGCTCACTCATCTGACAATATCTACTTCCACAACTGTTTTTTTTTTCT |
| | TTGTAAGAGGAAAAAGTAAATGCAGTCCGAAAGGATCTGTAGCCACACCCTAGAAAAGGTCCTG |
| rs1037448 | GAGCCCTGGGCATGTGAGAGTTTCATAAGGTACTCTTGACCAACCTTATGTGTTGAAAAACAGCCTTGTCCAAGAGTCAGGGGAC |
| | TCTTCAGAACATTCAATTCACCTTTGCCCCAAACAGGCCTACCAGGGCTAAAACAGGTAGTTGAATGTGTCTCATCATTAGCTAT |
| | TTGGGCAGGTGGATGTTGATACAGGTTAT[C/T]CTTAGGCATCAGTGCCCCATGGCTAATGCTGTAGGCAGTGGATGTTTTTGGA |
| | AGTAGCCAAGCAGGATTGCTGCTTGCCCTTGAGTGCTCCATGCCGTCTGCCNAGCTCCCATCACTTTAACTAGGAGAACCGGCCT |
| | CCAGGATCCCTCCCTTTGGTTCTAACATAGGCCTCAGATCAGATCCAGTGTCTGTGGTTTCAG |
| rs2244057 | TTGCCCCCCATAGTTTCTGAGAGGGCTCTTCCTGGCCCACTGCTTTAGTTACCACTCATATTCACATCTCTCNGACTTCTCTCCC |
| | ACGCTTTTTCTCAAGATATGTGAGATAAAACTCAACATTTTCCTTTGTGAACATGCCCCTCTGCCTGTATGCCTCATCTTATTCG |
| | ATGCTAACTTCTCTACCCAGTCACCGAAGA[C/T]AAAAATGCAGGCATCATAATAATCTTTCTTCTTTAATTATTCATACCCTCT |
| | CCTAGTCTTTATCATAGTGAATTGATTAAATAGTCATGCTCATTTCCTACTAAATATTTTTTGGCATCTCTCCATCCTCTCTCCT |
| | CTCACTCATTAGTTTACACTTTTTCACCTTCTGCTAAAACCATTGCAAGGCCTTCTTAACTGAC |
| rs10997044 | TGTACCAAAAGTACCCTGTCTTTTGCCCTCTCTTCTGAGAGAAACATTGCATTTTTCTGCAATATCTCTTCCTCGTGTCCTCTAT |
| | CCTACATTTATAGAGTCTCAAATATGCTCAGCCTCAAAATTAAGATGCTTTGAGCTTCCAGAAAACACTACACAGATAAAGGTGG |
| | CTTTCAAAAGAGATGTTTCTTATTGTCTAC[A/G]TAATCATGCAGAGTTAAGCAGTCACTGCAACCTTTCATCATTTTCTACAAT |
| | AAGGCCAACTTTCCAGATAACCACTTAGTGAAACAAGAATCCTGTATTTCTATGGTGGAGCTACTGCGGGTCTCATCCAACCTCC |
| | TGCCATGTTTGGGCCACTGACTGTTTGGGCACTTACGTCCAGCTTGCGACGATGGAGCCATGGG |
| rs9949324 | GTGGAGTTTCTCTGTTTTCCGAGACGGCTTACTCGGCGCCTAGTGAGNCGGTGCTTCTGTTTTTGTCTCTACTCCGTGTTCTGAG |
| | TGCACCCATTTATNCCCAGATAGCGTTTATACTTACAGAGGAGACAGGCGAACACTAAAACACAGCAGAAATATGACGAAACACC |
| | CTGCAACCTTGTTTCTTTTGAGTTCCTATC[A/G]CTCACTTCAATCTATTCTAAAAATAAAGGACTTGTCAGGCCTAATGATAAG |
| | GGAGAGTTTTAGGGAAATAAAGAGAGCTGAACTGACACAGACATAATAATGTCAAGATATAAATCACAGAGTCCTTCTGCCATTT |
| | TCATTATCTGCCTCGGAGGATTACGGTGCTTGCTATAAATGAGACGCTATGGGGACTCTGCCCN |
| rs618670 | ATTAAATACCAGTTTATGAATATTGCACCNTTAGGGCATTATCTTTTGCCAGCTTTCCTGCTCACCACTNCCTTGTCCTTTATTC |
| | CGAATGTGGACACATGGGTAGGAGGCCTGCAAACCCGTGATGACATTTCTCAGTGTGTGGGGCCATCTTGGATCCTGGCAGTGGA |
| | TGAGAAAGGCGTGAAATGGCAAGGAACGGC[A/G]ATGGTCAACTGAACAGTGCATGGTTGTCCGTGGGTTGAGGAAACGTGATGT |
| | CCGTGGATGTTGTTTGCTCTTTTCCAAAGATGCAAATGCNGCTGTGAATACGGAACCACAATTTGACCTAAAGACATGTCTGCAC |
| | TCACATGCCAAAATAGTTTGTCATTTGCTGTCCTTTGATAATTCCATGTAAAAGACTATCTCAC |
| rs4799159 | TTTATTCAAACCAGAGGACAAGGAAAACTACTGGATGAACACGGTTGTCTTGGGAAGAGACCTGCAGGAAGCTGCCCCAGCAGAG |
| | GTAACCCGGTACTAACAGCTTGATTCTCATAAAAAGGTGGCAATAATCGTGAAAGTGTGAGAAGGAATGTGGCGTTATCCATTTA |
| | ATCCCAGAACTGAGGCTGGGAGTTGTCTC[A/G]CAGTGACGTGGGCCAGGCCTCTCCCCAGCAAGGGCGATTAAAGTGAAACCAC |
| | TTAGGACATTTGGTCTATTTCAGATCCCTGCCGGTAACCTTTTCCTTCTTTTCACCAACTCTTTTCAAACTTAGGTTTTTTTTTC |
| | CCTTCTGTCTTTGTATTTTCCCGTAACATAGCCCTCGGGGATGTTTACTTAGATTTCCCTGTT |
| rs10490832 | AAAAATTTCGGCCAAAGACAGACTTCAAATAAGAGGTCATTGTTAAGGGTTTTAAATTCAAGGAGACAAAGTGGCACGGTAACAC |
| | ACATACACAATTTAATCGGAAAAGAAATTACCTCATAGCCCAGTAAATGTCCATTGCTCAACTTCCAAGGAATGGTGTTCCATGA |
| | AACCTCAATTTCTGAGGAAGATAGGCTATT[G/T]GCAGAGACTTGAGATGGGGCCACTGTAGGCTCTGTTCGGAAACAGAAATTG |
| | AAATATGATGATAATTTTGGCAGTTAGTTTAAAACACAGATTAATGTTCTTATTTTTATAAAAGCATTTTAACGTATTTGAAAAT |
| | TGTATGAAATATTCTGGCACCATATGTTGAAAATAGAGACACTGGTCCATTTCCGTTTCTTAGG |
| rs1545384 | TTTAGGGAGCAAACCAAGGTGGTATAAGAGAGTAGATTGCTACAATGGAATGTATGGAAATATAAACAAGTTANAAACCATACAT |
| | GACTGCAGTCAAAGATTTCTGTAGCCAAATAAACAACAAGCTTCCTCCTGGGCATTTATGGTTTCAAAAATAAATAAGGGCAGAG |
| | ATTACAGTCTCAGAGTCTATTTACTTGCAC[A/C]AGGGTGACACTTCTTTTTATATCCACTGCTCAGCACCAGCTCAACTCTTTT |
| | CTCTGGTTCCAACCCCTTTGCAATTATCTGCCTGAGAAGAAGAGGGACACATGTGGGTATCTAGTGGGAGATAAATGCAGCATGA |
| | CAAGCGGATTACCATGGTTGAAACACTTTTCTGAACACACTGGAGCTTAAAAAGCCTAAAGCTN |
| rs649867 | TGTGGGGTGTCCCTGTGTCTCCGGGGTGACTTGGGCCTCCAGAGAACCCCTGGCACAACCTCCAGCTGAAAACAGGAACAAAGAA |
| | AGTGAGCTTAACATTCCGGCTGTTTCAGACAATGTGTTTCTCTTTGTGGGGGCAAGGAGGGCAGGGAGACACCTGAAACCATTTT |
| | TATTCATCAGCTCAAGTTGGTGGGTGGCAG[A/G]GTCATGTTGAGGGAAAAAAGCCAAACTCTGTAAAATATTTAGAGAGGTTTC |
| | TTCTGAGCCAAATATGAGTGGTCAAGGCCGAGGCACAGTCTCAAGAGGTCCTGAGAACATGCGCCCAGGTTGGCTTCAGACATTT |
| | AGGGGGACAAAAGTTACAGGCAGACGCCAATCAGTACATGTGAGGTGTGCGTGGGGTTGGTCT |
| rs2279103 | ACTTTTCATTATTATTTTTTGTAAATTATGCATTTTCACTTGTAGAAATCTCTTTCCTTGTGGAGACTCAATGGTTTGCATTATT |
| | GATGATCGAGAAGATGTCTGGAAGGTTTGCCCCCAATCTGATAACTGTGAAGAAATATGTATACTTCCAGGGCACNGGTGATATG |
| | AATGCGCCCCCTGGGTCCCGAGAATCTCAGA[C/T]GAGAAAGAAAGGTGGGTAACCTCCTTCCTGATTCTCTAGAAGAATTCACA |
| | TTTGCTTATTGNTTAGCTCTTCTTATTTCTTATCTCTGTTTTGACTGCTATAAATTCAAGATACACTTTTTTTATTTGTGTTTCA |
| | GTAGAGATTATTGGATTTATTTATAGAGTACTGAAAAACAGGATATTAGGTTGTTTCAATTTGGG |
| rs523386 | GCTGAGGCCAAAACAGGCAATTTCCCTTACTCATGTCCTTGGCGTGAAGNGCCTCGTCCTTGTTCAGTCTTGCACCGAGAGGAGA |
| | GCTCTCTGAGCTCGTGGCCAGCAGGTTTCCTGCTGGAAGCCTTACCTGGATGCTGGGCAAAGCTGTCAGAATAAAGTTCAAGGTG |
| | GTCAGGGCTCTGCAGGGTCCCTCAAGGGAG[C/T]TTCCCTCATCTAAAAACATGATTCAACACAGTGAGCGATTTTCAGTTGTGG |
| | GGTTCCTAGCAGTGTCTAACAAGCCATTCTGGAGTCCATCAACATTTTTTTTTTTTTTAATAAAAAAGGTGAGATGGAATTAAGG |
| | CTGGAAGTGGCGGACTTTGGGAGAAACATNCCAGGAGTCTGGCCTCCTGNGATGAGCAGAGCTC |
| rs7704939 | GAGGATGCAGAAGGAACATCTGGGCTCAGCTATTTCATGCTGTCGGCCATACACTTCCAAGTGGGAGGTGATGGCAGTTTGGTCA |
| | GCTGTTTGCCAACCCAGGAGTTGGGTAAAGGTTGGTAAGAGATGTTATTAGTTTTAGCACCATTTCCTTCCAATAGTCTCATTTT |
| | AATACAATACAGGTTAGTAGTGACCAGAAA[A/C]TATTGCTACTGAATTGAAGGATGACTATACAAGGACTTATATCAGATTATT |
| | TGTTGCTTTTAGTTTCTAGTGTCCCATAAAGTATAGAAATGGAAATTGCTGACAAGAGAGTACCTTTGGTAGGCACAGTTCCTTT |
| | TCTCCCCCCACTGGAGAGGACGCTACAACCTTGCTATAGAGTGTCCTGTGACTGCCCAGTGATG |
| rs1958438 | TATCACTCCAGATGTTCTGGACAGAACAAAGATAGGCTAGAATATGTGATGTAATGAATTAATGAATTTTTTTATACTTCATCAG |
| | CCAACATCAAGATAAACTGTATTGAACTAGCATGTGTCCAGCTGAAGAGTGTTTAAACTGTTAGTGCAAAGCTGAAACTTCAATA |
| | AACGCTAGGAAAGGATCCAGGTTTTATTAC[C/T]GGTGATACAGTTTGAGTGGATAAATTCTAACCATTTTAGCTAAACCCTCCA |
| | GCTTCATATTAATCATGAAGTAGAATAAAGTGGGGAAAAAAAACAACCATGGGGAATGTGAAAATAAGAGTTTAAAACTGAAGTA |
| | TTACAAAGGTCAGCTTCTTTATGAGAAGACAAACATAGCTGCATTTTAATTAAATTATGAGATT |
| rs1958421 | TTATCTTTCAGTAAATTCCTAATGAAATAACAAAGTGTATTGGGTTATCTCCAACTGCCGTACCATACTACTCTCTAAANATAGC |
| | AAGTCAAAAATGACTGACTTTGCCAATATCCTGAGATTCTACAGACATCTCAGTGAAGGCAGAGGATGGTGGGGAGAGTGGCATT |
| | TGGCTCAGCTTCAGAACTGCACTGTCTCC[A/G]TGATAAAGGGATCCTGTTAACTTCTGTTTTCCACCTCCTGACAGTTGACATT |
| | TTAATGTGTGTCAGGAAAATCCATCAACATGCTCCTCTCTCAAACCCAGGATTTCGCNTTGGGATCAGTAGGAATCTGATCAGAG |
| | CTAACACAAGCCCTCCTTCTCATTAAAGCATACCCCATTCCCATTGTTGACTGTCCTAATTT |
| rs9323737 | TATTCCTTCTCATCCAAAATTTGGTTATTTAAAATCTCACTATTTGAACTCATGAATCAACATGTTTGGATATGTTTCCACATAA |
| | AATAACTCACTACTTGCTATTTCAAGTTCAGGGTAAATTTTTTAAAAGTGTAAACGATATCCTACACCTCTTTATTCAAATTTAC |
| | TATCTAAACTTGTGCTGTCCAAATTCAGGA[A/G]GTAGGTAGTTGTGGGTAGCAAGGAATACTTAAAGTGGATTGTAGCTCAAGT |
| | CATACAATAGCAGCATTTGTTTTCACGATCCATTAATATCATCTCTAGTTTACAACCATCAGGAGCCTTTTCATAGCAATGAACT |
| | TTCCTGTGATAACTTTGAAAAACAAAATCTAAATTTGTCATGGCCTACAATGCAATATATGAAA |
| rs12142266 | TCTCTCCACCAAAAGGAACCAGGCCTCCTTGGAGAAATGGCTGATTCCAGGATTGGGGCAGGGAAATTACAAGATGAGTCTAGAC |
| | TATCTATCTTTTTATGGCCAAAAATAAGGAGGTGTTCAAGCAAGATGAGGACATGTTAAAAGGACACAAAAGGCAGCTTGAAGGG |
| | AGCTCCCACTGGTCAAATCTGCAACATTCA[C/T]ACATCAAAAAATGCCAGTAGTAATAAAGCATAATCTATTGAACTAAATAAG |
| | AAACCCATGAGTCCACATTGGTATAAATAAATGAATTATTNCATAAAGGGGGAGGAGAGAAACTTCCTTTGTGTATTAGAAAGCC |
| | TACTAATAAATGTAAAAGGAGTTATAGAGTTAGAAAATCACCATTTGGCATCAGTAATAATTAT |
| rs6500497 | AGGGCTCCCGGAGGACAGGACCTTCCAGGGCCTCCCTGCCCCGAACCCATAGGTGCCAGGCTTCTCACTGCTCACGACCTCCGTG |
| | CCGGGCTCATACCTGCCGCGCCAGCCCCTCTTCAGCAGAGCACAAGTCTGGCAGCAGGAGGGCACCAGCCAGGGGCCCCGCGCCC |
| | CCGCCTCTGTACAGCAGGGTCACCGTGGGG[A/T]GACATGAGAACACCGACCCAGGGAATGGGCTCCTGCCACACCTGCCTGGGA |
| | ACCTGGNCTGTGAGTCCTGCTCGCTGCACCCTCTAGCCTGACGTTGAAGTAACCTCGCCAGCGGGGCCCTCTGTGGCACTGGGCA |
| | CTCAGGGAGAGCACGTGGCATTGGAGAAGGTATTCTGAGTCCTACAGAGAGGGACCTGCGGGGC |
| rs6500498 | GGCTTCTCACTGCTCACGACCTCCGTGCCGGGCTCATACCTGCCGCGCCAGCCCCTCTTCAGCAGAGCACAAGTCTGGCAGCAGG |
| | AGGGCACCAGCCAGGGGCCCCGCGCCCCCGCCTCTGTACAGCAGGGTCACCGTGGGGNGACATGAGAACACCGACCCAGGGAATG |
| | GGCTCCTGCCACACCTGCCTGGGAACCTGG[C/G]CTGTGAGTCCTGCTCGCTGCACCCTCTAGCCTGACGTTGAAGTAACCTCGC |
| | CAGCGGGGCCCTCTGTGGCACTGGGCACTCAGGGAGAGCACGTGGCATTGGAGAAGGTATTCTGAGTCCTACAGAGAGGGACCTG |
| | CGGGGCCTGCCCCACAGGCACGTGGGGCTCCTGCTGGGGCTGTCAAGGACCCCCGGGCACTGAG |
| rs1454333 | TACCGATTTGCAACAAAATTTACTTTTCATGTACTTATGATTCTAATCTATCTCAAAAGTAGATAATCAAACTTTTGTAAATAAC |
| | CTTTCAATGATGGACAATGTTATTGCTCCTTTACCTATTACTACCTCTCCCTGTTCTTTTCTTCATCCCAATCAATATGTCTAGA |
| | TAAATACATTTTTTTCTCATTTGAAATTTA[A/G]TTTGGAAAATGCAGCCACATTTTTACTTATCTTTTTCAAATAATTAAAAAC |
| | TTCTACCTCACTCATGTAACATCTTCCTGCATGATCTAATCTGCATCTAATTTTTCTCTGACTAGCAGCACTAGATGTTTTGTGA |
| | TTAGGGAAGTCAGAAGTGATGAGACTTTAATAGAACATAAAATATTTCTTTTCTATATTTCAGT |
| rs9589698 | ATATATATAACTTAGAAAATGTTTGAATGGTATGATTAGAATGGAGTCATAGTGTTTTGCAAAACATGGATTGTTGAAATACAAA |
| | AATCCAGGTTTTGAAGATTATTAAATAATTCATGTAAGATATCAGAGTGGAGCTCCAACAATTTGTTCTACTCTTATTTGGATAT |
| | AATGTCTGTGAGTGATTCCGTAAATGAGGT[C/T]ATAAATATTCATCTATTTTATCACTGGCAATAGCTGAGAATCTCTGAACAG |
| | ACAGTGGACCACAGAATTCAGACCCTCTATGCCTGTGCCTATCCATTGACTTATCCAACTTTATTTCCATGTTAGATACCAGCAG |
| | TTCAGAAACATTTGTATTTCCCAGCTGCGGAAACAGAATTATGCTTCTTTCTATTAGGATTTGA |
| rs2025949 | GACATCTCAATTTCCACCCTCTTCATAAAATAAACAAACACACAAACTTTGAAATACAATAAAGCTATTAGTTAACCACTGGCAA |
| | GTTTGCTACAAAANGGCTTATCAATGATGAATTTGCCTTTTGATTTATTTATCATTGTGAAATACTGTCAACCCTCATTTACAAA |
| | TTAGAATTCCTAAACCAAACTGGTAAAACT[A/G]AGATTCTTAAATTGAACAAAACTATTAAATGCTATACTGTAGCATTAAATA |
| | TTTACAGCATTTTAACTTTTATATTTTTGTATGTAACACTGAAAATTTTTATAATGGCTTTTGTTCTAATTTGCATATTGAATAT |
| | ACAAACGCTGAACAGGAGAGAAATTAAAAGAAGTGATGTCTTTTCCAAGTTAAAATCTCTCTGG |
| rs902923 | AATGAAATGCANATGTAGTAGCCAGAATTTGAGCAGTCGTTTTGATCATGAGGTGGAAGCAATGTGCAGAAGATGATGGGGANAA |
| | AAGGAATTTCAACAGATTTTTGGGAGAGAAAAATCAGATGATACAACTGGTTTAGAAGGGGGAAGAAATTTACAATCCATGGTGC |
| | CTGTAGAGATTGAGGCTAATGGGAAGAGA[A/G]TGTTCTGCCTTGCAGAACTTGTGAAATGCTCAGGAATTTGAGGCAGTACATA |
| | CAGTGGTCAGCTGGATTAATGAGGGCTGGCTGGAGACTGTGGAACAGTCAGACTCTAGAACACCTTCACCCTCCACACTCAGGCA |
| | AGTCACTCCTCCAGGTCCTCCAACCACTGCCCCTANAGATGAGATGAGTATTATTCACTGGAG |
| rs1984151 | GACAACATTCTGATCAGNTTTTCTTTTCTTTTTTATACTCCTTTAATTTTTGCAGTTTTCTTTAGATTGTGCCACCTCCTGGTCT |
| | AGCTGTTTCTTTGTGAATTTNGTAATAGGCTCTCTGTTTGAATTCTATGGTGTTATTCCAAACATTGCTGCCTTCTTTTTGAAGT |
| | ATGTGGCTCACATTTCACACAGATCCTTTA[C/T]GATGATTTCCTTGCATAGTGGGAAGTTTTTGACGTGAAGGTTTACTGGAGT |
| | CCCTTGGCTTTCAAGNTGGCTCAGAGCAGATGGTACTGCTAGAGAGAGCAACAACACAGCTTTGGGCTTGAAAATGTTAGTGTAC |
| | ACAGATGCTTTTTCCCCTCAGGACTGGTATGATTGATGGGACTTTGGAACTAAAAACTGAGAA |
| rs1459841 | TTTCCTTTTATGGATTGTATTTCATGCATGGAGTCTAAGAACTCTTTGCCTAGCCCTAATTCTCAAATTTTCCTTGTGTTTTCCT |
| | AAAAGTTTTGTAATTTTACATTTTACATTTATGTTTACAATCCATTTTTGGTTAATTTTTCTTTGAGGCATGAAGTTTTCTTGCC |
| | TATGTTCATCTTTTTGGATATGGATGTCCA[A/G]TTGCTCCAGGATTTGTCAAAAAGACTGCTATCCTCCCACCATTAAATTGTT |
| | TTTGTACCTGTGCACCATACGAAACAGCACATTCCTATGGGTTTATTTCTGGGTTCTCTATTATGCTTCATTGTTACATGTGTCT |
| | ATCTCTCTGCCCATGCCATTCCAAATAAACCATAGAATNGGTTTGTATATTTAATTAATTAATT |
| rs4917639 | TTATTTCACTTAGCCTAATGTTATGTTCTCAGGGTTCATTCATGTTTTTGCATGTGTCAGATTTTTCTTCCTTTCTAAGGGTGAA |
| | TAATATTCCGTTGTATGTATAAACCAAGTTTGCTTACCCATTTATCTGTTGATGGGCACTTGGATTACTTTCACCTTTTGACTTT |
| | TTGAGTAATGCTATTATGAATATGGGTAA[A/C]AAATATCTCTTTGATGTGCTGCTTTCAATTCTTTTGGGTATAAATAACCCAA |
| | AAGAATAACCCACCACGCCTGGTTAATTTTTTGTATCTTTAGTAGAAACTGAGTTTCACCATGTTGGCCAGGCTGGTCTCAAACT |
| | CCTGACCCTGTGATCCACCCACCTCAGCCTCCCQAAAGTGCTGGAATTATAGGTGTGAGCCACG |
| rs9332172 | ATTTTAGCAAAAAAAAATATGCTGTGTGACTCAGCTAGCTGCAAAGAGCCTGATGAATGGAATTTTTAGGNAAGCATGGAATAAG |
| | GGAGTAGGAAATAAAGTTTGGGCAAGTTGGTCTACAGCCTCTGCTATACAAGCAGTATTTTTTTCTAGTACTGTACTTTCCAGTT |
| | TCTATGTTGGTAACTATATAACTATGTGA[A/G]TAATTTTGAATTCACTGTAATCAAATATGCTGGTAAATAATTTGTCAGATAA |
| | TTGCATCAAATCATTCCTAGGAAAAGCACAACCAACCATCNGAATTTACTATTGAAAGCTTGGAAAACACTGCAGTTGACTTGTT |
| | TGGAGCTGGGACAGAGACGACAAGCACAACCCTGAGATATGCTCTCCTTCTNCTGCTGAAGCA |
| rs9480684 | CCCCAGTTTTACAGACAAAACAACAACAACAACAACAACAAAACCCCGGAAGNTCAAAGGGGTTGGCCAGTTTGGCCACAATCTC |
| | ACAGCTGTAACTGCTGAACCAGAATCTGGCCATAGGCTGACTTCAAACATTTATTTTATTATAATAGTGTTAGGCTAAAAATCAC |
| | AAGCTGAAAGGCCGTGATTGCACTTAGTTC[A/G]GTAGAGTTGATGATTCTCATGTATACACGTGACCTTTCATTGAAAAGATCT |
| | AANATCTCAAAGGAATTAACACAGCCTACAGGATTTCAATGCAACATGTGTTCAATGAAGCTAATAGGGAGTGAACTGATGGTGC |
| | TTGGGGGAGAATAAATTTAGNGTGGTTGTCCTCAAACTTTATTTGCATCAGAATCACCTGGAGG |
| rs2348427 | CAATCAGCAGAGGACATAGTACTTGGAGTTATTTTCTAATAGTGACAGTGAGCTTGAATTTCCAATATGACTCTATCACCAGGTC |
| | CTCTGACCAAGGACATTTACTTGTAATATTATACTAACTAGGTAAATCACTAGAGCCTGTACCAGTTAGTTAGCTGGGTTCTTAG |
| | GATGGATTTTATATAAAGGATTTGAAGTGA[C/T]ATAATGAAAAAATACAGTTTTGCAAAAGATCCCAAAATGTCCTGTAGGTGG |
| | TAATTATTTCTTAAATGGTTCTTCTATGAAAACTAAGAGCTATAACATTTTGTTTTAAATAAGTGATGCATTCCATCATTTGGGA |
| | GGGAAACAAACATAATATTGTTTTAATACATTACTGTCCAATCATCTATTTTATAAGAGATCAAC |
| rs2774089 | CTTTCCCTGGGTGTGGGNACTGCCGGTGTGGGAACTGGCTGCTGGCGTGGGAACTGGCTGCTGGTGTGGGAACGGCTGCTTGTCC |
| | CAGTGGTGCCCACCTGTGTCCACACTTCAAGGCCACCTGGGCCTGGAGATGTCACTAAGGGTTGACCCGGAGACGCCGACCTCAG |
| | GGACCTGGGAGGCTGCACACTGGCCGTGTT[G/T]GAAGTGACCCTCATGTCACCCAGGTCAAGTGCCACGGGCCCCACACATCCT |
| | CTTCTTCCCTGTCTCCCAGCTGCAGGCCCGGACTGGATGGCTTTGGCTAAGGAACCAACGCAGGTGGAGAAGGAGGCTGACGATG |
| | GGAGCCCAGGCACTCCAGCCTTGAAGGAGGAGAACCTGACCCTGAGCCTGCATGGCAGCCGTGCG |
| rs4907674 | GTGAACTAGTACACTTGTTTTACCAGAAGTCACACAGGATCCTTAAAGCATCCTCATTCTTTCTCNGTACAGGTTCTGGATGGAG |
| | GTCTCATGAGGGATTCGGGCAGAGGCTGATGAAAACATAAAACAGGCCAGTCTCCAGATTCAAGGGAGAGCTGGAGACAGCATTC |
| | ATAGCTTGACCAGGCAGCTTTGACCAGGTG[G/T]GACAAAGGAAGCCCAGATGTCCCAGTCTATTCATGTTTCTATCACAAAATG |
| | GCCTTTGAATTAGTAATTTATAAACAATAGAAATTTATTTCTTATCGTTCTGGAGGCCGGAAAGTCCACGATGAAGACACCAGCT |
| | GGTTCCCAGCCGGTGGGTGAGGACCCAGCCTCTGCTTCCAAGATGGCACCTCTGGGCTGCATCCT |
| rs9877859 | CAACAAGAGATAGATATGCATGTTTGCTGCATGCCAGATATCTTTTATTTAATTCTACAGCTTTTTCCAGAATGGCTGCCAGGTA |
| | GAGCACCTGCATCATTTTGGCAAAGTCCTCTTTGGAGTGGGGTCTTCAAGCCTGATCTTGGAATTGCGTTTTATTATTGTCTGCC |
| | TAGGACTCAGGTCTGCACTGTGGGACACAA[C/T]GCTAAGTGGAAGGCCCCTTATCCATGGCACCATGGAAGCAGGCGCAGGATC |
| | ATGGGAAACTGCAATGGCATTAAAGGAGGAAGCTCCAAGTGGATTTGGAGCCATTCTATAACTCCTTGAAAACAAGACTACCTCG |
| | GGGATATTTAGGATGACTCGTGTGTCACCAAGTGAGAAATACCTAGTGTTGGAACATTTCCAG |
| rs1204798 | CTAACTACAGATTTAAAATCATTAATTATTGTCATACAAAAGTTACCACATTATAAGACTACACTGATTGTCAAATAAAGTGTCT |
| | TCTTCACATTTGAAGCACTCTCTGAAAGATGACCATGACTTGCTATAATGCTGCTTTCTGTGGGACCCATGTAACNCACAAAATT |
| | GTTAACTCTTCAAGCTAACAAAGCCAGCCA[A/G]TTTCAGGCCCCCAAATTTGAAGATAGATTTTCCAAATATTTCCAGCTGTTA |
| | GAAACTATTGAGTTCACTACCAAAGCCAGCTTGGTACNTAGAGTCTNTGTGCTACTCTCTTTTTTTTCCTGATTATGTTTATACT |
| | TTCAAAGAGTTGTCAGGAAGACTAGTACTCACCCACCTTCTATTTCCAGTGGAATGTTCACCTTA |
| rs7719325 | GGGATTCTCAATCCAAGTGCTGACATTCTGGCTAAAAGAGCAGTAAGAGATGGATGATATAGGCACAGTCTTGGCATGTCTGAGC |
| | CCTGTGCCAGAAGGTTATCAGCCCTTTTGGGGGAGATAATATCTGCAGCTTTAATTTTCTCTCTGACTACCAGGCATAATGTAAA |
| | ATTAATGTGCATCATGTCAAGGTTATGGCA[A/G]GGCATTGTTGTCTGGAAGGGAAAACTGTTTGCTCCCCCGCCCTCCAAACAC |
| | TTGCACATACCGTGATAATACATTATTAAAAAGCACATTTCTTTGTCATTGTCAATAGTGGTGAGCCAGTAAAGGATATTTTCCA |
| | GCACTCATTATGCAGGAGGAAGTTAAGAGACTTCTAAAACTCAGTCTTAAAACAAAGAACATAC |
| rs6873640 | TCCCCTCTCCAAAACTATGAAAGCTCAGGTAACAACAGGCAACAGCAAAATCACCCGATGCTTGTTGGAGTCCTGACAACGTTGA |
| | CAAAGTCCTTGGCAGGCCCTTCTATTTTCATGATCAATGGTACTANAGCCAGAAACAAGCCTCGCTGGCTGTGAGCTGATCTCTA |
| | CTGTGAAAATGCACGCACGGTGTACACATT[C/T]GTTTATATTCTAAGCACTGAGGATGAAATAATTCTGCACCGTGTCCATTTT |
| | TGTGCTGTTAAGGCATACGGTTGCTTTGCAAGCAAAACAGTAGATCTTTCATTTGCCAGTGACATTCCACAAAGTGCTTATAATT |
| | CAGTGTTAGCTTATCAGCCGTCCAATCTCCCACTCCAGGGAATGTGTCAACCATCAAAAGGAGC |
| rs2980872 | GGGGGACACGCAAATGTGTCTCTAAGTCAGGGCACCCTCTGTGTGCACAGAGCCATCGAGGGCACTCGCCTGGCTGGACCCCAGG |
| | GAAGGAAATGGGATGGCTTAGTGGAGCAGCCCAGTGTGACTTCCCAGAGCTGACTTCCCAAAACATGCCACGTCCTCATCAAATA |
| | AAATCAAACCCAAACCCCTCTAGGCTGCTG[C/T]GCCNGACCTTGCTTACTCTAACCTCATCTCACCATCATGCTCCAGCCATGC |
| | CTTCTTCTCTATGTAATAGTGCTGGGCACTTTCTGCCACAGGACCTTTGCACACGCAGAGTCCTCTTCCAGGGGCCTCTTCCTCA |
| | CCACCCACCCCTCTGCTTCTCCCCACCAACTCCTACTTCCCCTTCAGGTTTCAGCTACAGTCCC |
| rs554710 | CACAGAGAGAGGACTTCTGTCCTGATAGACGTCGGTGAGGACTGAATCCCCACTTACAGGCGTGCACATCAGGGACTGATGGACA |
| | TCGCTGAGGACCGATCCCAGCTTACAGGCGTGCACACTGGAGGCTCNGAAGAGCTGACTGTGACTCACTTCCGGCTTCCCCCCAG |
| | GACAAAACCTGCCTCTCCTTCCAGACTCGC[C/T]GACTTCCCTTCATGTCCCGCTGTGATGTGCAGTCCAGCATCCTTGGGTCAT |
| | GACACCAGCTGCACTGGCACAGGGAATGAGAGAATATTCCTGAAAATGAAGACTACCGCGGAAGGCAGGAGCTTCTAAGCTAGAG |
| | TGACTGGCAGCTTCAACAGGCCCTGCTGCACCTGAACCAGCCCGGGGCCCCCCAGTGCCNCGGA |
| rs720733 | CAACTTTATAGTCACAAAAATTATCTAGAATTGCATCTATGATGCCCATGTTCCAGGCAGCAGAAAAGAGGGCAATGAAAATGAT |
| | AAATGTCCCCCATTTAAGGACCTTCCCAAGAGTCTCAGGTATCAACATTATGGACATTANATTACTAATTTTAGCTGCAAGGGAA |
| | GCAGGGAACTGGAGTACTTTAGCTGGGCTG[A/G]TCTGAATACAATTGTTTTTCTGCTATTCTGAATATTTATTAAAGTCTTAAC |
| | TCCTTACTAGGTCTATTTCTCTTTTAACAATTGGGTCCACAATTGATTCTTCAGGATGTTTATTGTGTTTGAGGAATAGCTAAAG |
| | AGTCATCATGGTGGTTGAAGAGAAGCAGTCAGTCCATATGCTCATGCTTTTACTAAACTTGAGG |
| rs4388301 | AAGACATGCCTGCTTCCCCTTCACCTCCACCATGATTGTAAATTTCCTGAGGCCTCCCTAGCCATGCTTCCTGTAGAGGCTGTGG |
| | AATTGTGAGCCAATTAAACCTTTTTTCTTTATANATTACCCAGTCTCAGGTATTTCTTTATAGCAGTACAAGAACAGGCTAATAT |
| | ATCCTCTATTTATCTTCTCTCATTTCAAAC[C/T]AAACTTGTTCTCCTTCATCAAAGAAAGAGAGAACCCTGAAATTCAAAGAGA |
| | CACAAAAACATTAGTTTTTGCTGTTGTGCTTTGATAGGCCCAGCTATATTGCTAACGGTAGTTUTTGTAAGCAACTACTGTTACT |
| | TGAAGGGTCATTTTGACTATCTACTATTTTTGCCTTTTTAGTGTATTTAGAACAGGCCATTCTT |
| rs1109089 | TTCTGTTCATTAGACGCACGTCACTAGGTCCATGCAGGGAGATGACACACAGTGTGGCCACTAGGAGAGCAGGAGATCTGGGAGC |
| | CGGCTCCAAGCTGCCTGCCACACAGTCCTAGTTCTTTCAGCTGTTTCTCACGTGACAGGGTACTGTATTTTTGCCCAAGTGGCGA |
| | CACTTTTAAAGATAGGCAAATACAGCATGC[A/G]GAGGAAACCGTGGTTCTGTAATGCAACCGCAGGTCCTTAGGACACCCATAT |
| | CCCATTTGTTGACCCACAGCGAGCTTGTAGGGGGGTTAAATTTCATTTTTGTTTTTTATCAAATGACAGCACACATCGTTTAAAA |
| | TAGCACAGTGTGTAATTCATTCAACAAGCTAACTTATTCTGAAGCTTGTGATGCAATCATGAACA |
| rs2074997 | TTTCCTCAGACATACTCCATAGATATTAATGGCTATATTCTTGTGTATTCTGTTACATCAATCAAAAGGTAAGACTCCTGCTGCC |
| | TGCTTGAGTTGATTTGCTACTCATAGATAACATTGATCCTTTTAAGGGACTGTGCTGGAATTATTAATAGTGACAATTAGAATTA |
| | TTAGGCTACATGTTTTTTAGTGATTTTCTC[G/T]GGAGCAACTGGATTGCTGGGNAACATGGCCCACTAGATGTTCTGTTAGTTA |
| | CACCAATAGGATAAAAGGCTCTTTCTTACCTCAAGCTACCTAGAAAAACAGTGGTTCAGCATGATCTAAGCTTTTCTGCTAATTG |
| | AAATGGGAAATGCCTAAATTAATTTTACTTTGTGTGTGTTTTTACCATATTAAGGTGAAATAG |
| rs965118 | TCCAAGAGCTGCTTTATAAATTGGATGCATACGGGGGCTGGTTCTAGGATTAAGACTGGTAGTAGTAGGGTAAAATGATTAAGGG |
| | GCAAATTCTTTTAAAAGTAATCTATAGTAAATTTGTTCACTGGCTTCCACATTTCAGGAGTGAAATTGCACGCAGTGAATTGCTG |
| | CATAGCGGTATTTGTTAGCAAGTTCTATTT[A/T]CTACTTAGTAGTTTATGTCGGTACATCCATACCACTATGGTTTAATAGTGG |
| | TTTAATTGCCCACCTAGGAATGATGATGAACATGGTTAAATACAGATTATTTTTAGATTGCATGAAATATTTTGGTTTTGCCCAA |
| | TTTTAGGGTGAGAACATATACCCTAAATGAATTTTGGGGTAAGAATGTGAAGTATCAAAAAAAA |
| rs3753151 | TAATTCTACTGTCTCTCATTTTAACCTCATGTCAAAGTGGAAAGCTGGCTTTCTTCTGACATCACTCTCCTTGAGCCCCTTTCTC |
| | CCTGCNTCTTTGAATCACCAGAGAATCCATTTCCAACCTAAACCCCTGAAATTCTCCTAAGTAGACTAATAGCTATTTACTAGTA |
| | AATGCTACTAACCACGGTCATAGAGGTTTC[C/T]AAAATTTGGTGTTCACAAAGCAAGATTTGTGTTACTAAAGTGTGGTGAGGG |
| | CTCTTCATTCATANCCTACTGAGAAGTCCACACCCAGGACGCAGTGCCTTGAGAATGCTGCAACACCTAAGTGCAACCTCCCAAC |
| | TTTCTGGAAGGCGCTTTGTCATTCACTAACAGTGCTACCTTGAGATGGCAGAAGTCGGAAATTG |
| rs2299965 | TACTCTTCCTCCAAACCAAAACCCACCTTAACTTTTAAAAATAAAAATTATTTTAAAAAATTCTTCTGAGCTTTTATCAGGGAAC |
| | AGCATTGTATTATTTTTTCCAATGATAAAATGGCACAGTGAGTTTCTCAGCTGGTATTTTCACTACAGAAGGTGTACTTTCAGGG |
| | GTGTTGACTCAATCTTATACCTCATGATTA[C/T]TGCAAAATAGGCACTGTTTACAGAATTATTTTAAGTTTTCAGATTAATATT |
| | GACTTTGTTATCATGCTTATTCTCCTAAAAAATAACTTATTAGCCCGGTGTGGTGGCTCATGCCTGTAATCCCAGCACTCTGGGA |
| | GGCTGAGGCCGGTGGATTGCTTGACCCCAGGAGTTTGAGACCAGCCTGGACAACGTGATAAAAC |
| rs2299967 | TAAAGTGCTACCCAGAAAGGCCATAAAACTGTTTCCCTGGAGATTTTCAAAACAGGATATTCATCTCTACAGGATGGTTTAAAGA |
| | CAAAGCTGAGGTGGAAGGCAACAATAATAGCCCTGATAAACTGCAGGAAATTAAATGTTTAGCAACAATGAAGAATGAGTTTTCC |
| | TAGAACGGTGGCCAATCTGGAGAGAAGACC[C/T]GGGATACAGGTGAGGGTGGGAAAGAGCTAGAGAAAAGCAAAGACAAAAATT |
| | CTCTATTTCTTAACATATTAAAGAACATGAAATTTAAGAATGATCAAAACTAGTAATAACACAGTATGAGGAAAATGAGATATGA |
| | AATAGAGTATACTCATCCGTCAGCCTGAATGGTTAAAACCATTCATATTTTATAAAGANAATTT |
| rs6948196 | TGACAACCAGCTTTTAATTATGGTCTAATGGTAAACTAGGTTACCAAAGCTCTCTGTACAGGTAAAATCCCAGTGAAAGATGTAC |
| | CAGGCTGAAGACAGTTTACGTAACACTACCATTTTAAGTTGAGTTACTTCCTATCGCGGGACTACTCCGTTGTGGTAGAAATAAC |
| | GGAATGTCAGGGAGCTGGTACTTTTGGCCA[C/T]CACGGCAACAGTTAATACAAAGCACTCCCTGAAAGCCAGACTCCCAGAGTT |
| | ATAGGTGACGTGTCCTTAAAAGCTTTNACATTGTAAATACCAGGAAAAGTACAGGGGACTGGCAGGGAGAATTGTATAGACTACC |
| | TGAGAATTATATTTTACCTATTGGTAGTTCTGACAACACTCAAAAAAAAAGAAAAAAAGAAAAC |
| rs12617566 | ACAACTAAATGTCAGTAAGCCTGGAAAACAACAGAGCTCTAGGGAGTTCAGCTGCAGGAGTTCATGGACTTTTACTCTAATGATC |
| | TTCTATTACCTCAGCTAGCAACTTATTAAGTTTCTGCATATCTTAGTTCAAATTCTCAAAAGGGAATCTGATTAATTCAGATTAG |
| | TTTATGGATTAGTTCCTCTGGGGTTGGATA[A/G]CTTCTCTTGGCTCAATCAGCCATGTCAGGGGAATGACATTGCTAATGAAGG |
| | ATAAACAACAGGAAACAAGATATTTCTCTAGTAGAGATATTTATGATGCTGGTCCATATTTATTTACATGTAAATGTATAATATA |
| | AAACAAACTACATGGATATTAATAAAATGTAATTACCTTATTCTAAGACAGTATATTTATAAGC |
| rs1421780 | CTATATTCTTCCCCTATTCTACCCATCTACCTTCCTTTTCTTACCCCAGCTACTCCAAAGAAAGAAAAATGGCCAGTTTGGTCAG |
| | ATATCAAACCTTTACAGGAANATGGTTNGCCAATGGAGGGATCAGGGCCACCAAGTGTCAGAAAAGCTATTCTGCTTTCTACCTC |
| | CCTCTGTCAAAGTCACCTTGCAAGACTT[A/G]CCTCTTGGGGCTAGGACAGCTGGCATCTCTCCAGTGGGGCACAGGTATATTAG |
| | GGGCTCAGTACCCATTCAGTCCCAGCCAGCCCTCTTCCCTCTGACCCGGCCTAAAGCCCACTTCCCTCCTACTTGCAAGAGTGGC |
| | TTATTAAGGGTCCCAAGATTTGAAAACAGGAAGAATGCAGGAGCCCTTTATACCTCAGTCTT |
| rs301191 | TCTGTGATTGAGGGCTGCAAAACAAAGTATGTTTAAAAATATGTTTATGTTATTTTGATAATTTTGAAAATGTACTCTCACCTGA |
| | GGATGATCTACAAAGCCAGTTCAGCTGGCTCTTCAGCACTGTATTAAAAATACTCTTAGTTCTGGGGAAAAAAAAAAAAACACTT |
| | TCANAAAACAGTTTTTGAAGAGTAACTGTG[A/G]TAGGTTCCGAAGTTGGTNTCTTTCCTGANAGGACTCTAAGTGCCCTANATT |
| | TTATATGTAAAATCATGTAGTTTGCAAGACATTGGAAAGGTGGCACTTCAATTAAACCCTTCACTNCCACCCCCAAAAGTTAAAA |
| | AATAAGTAATATCTAGAGAAAAACTGGTTATGGATCCACTGTGACTGGATAGTATTTGATCACC |
| rs301193 | CTAATTCATGGAGTCTGGGTCGGACNTGGCATTCTCTATTTTTAGCAAGCTTCTCAAGGATACCATGTTGATATTCTTCCCATCT |
| | TTGAATAGCAACCCATTGCTTTAAACATATTTAACATCTCTGGGCTACTATTTTCTATTCAATTGTTGGTCTACTCCACTAGGAT |
| | ATATGTGAGGTCCTTTAATTCAGAATAATA[C/T]CTTATTCAGCTGTGCAATAAGAAATGGCTGAAAAAATGATCATTCCTTCTC |
| | CTTTTTCATTATTACTTTTCTTTGACCAAAGAATGCTATTGCTCTAACAAGAAATGTACCAATTCTGATGAAGTCTGTAGCTTGG |
| | TAAAAATCACAGGGGAGGAATAAGACCACTANCTAACCAATGTATTTGCTAACATACCATAGAA |
| rs13403584 | TTTTCACTAGATTCAATAAAAGCTCACGCAAGTGTAAGTATAAGAGTGTTGGGCTTAATTATGGTTTGGCCTACTATTTCCTTTT |
| | ACTTCATGTTATATCTGAAAGGAATTTACTCATCATGGAGAAAATAAGCTACATTAACACTAAAACACAGTGAGAAACAAATATT |
| | GCTCCAATGCTAGAAAAAAAGAAAATTTCC[A/T]GGGCTCAAAAGAGNTGAGTTTCAAGGACAGTTGACACCTATACCCTACTTA |
| | CTAAGATAGAGTGAATCAGATATAGCTATAATTTGGCCTCTGTAGGTGTATTTCAGCTGTTTCATTTCTTTTCAGGGAAGAAAGA |
| | TGGTGTATGAAACTTTTTCCAAGATGGTCTCTGAAATGAGTTAAACTTCGGTGTTCAAATTGTG |
| rs7676106 | CAGAATTTCTANTGCTATACTTTCTTGAGGTAATTTTAAGAAAGGTTTCCTTANCATTTACCCAAATTTCATACACAGACACACA |
| | CCAAAACTAATAGCTGGGTACAAAAACTGAATGAATATTTTTGTGCTGATACAAACCGTCACAAAGGAAGTAATGATGGGATTTG |
| | TCTACATTAACCTGACATACTGGTGAAAATAAC[C/T]CCATTGTTCATTATTTGAATTAGGCAATAATTTGGAGAAATTCTTGAA |
| | GAGGNCATAAAGGAGACCGCAGATACGTGTGGGCTGCCTGTGAAAGGCATGCTGCTTTGTAAATATTCACACTAAACTTTCAAAC |
| | ACAAGAGTGATACTACCCTAGCAAAACCTAAGGTTCTCCNGGGAGGCAGGTGACTAGTTCCACTTTT |
| rs7685314 | NTTACTCTAGAAAGAAGATAAGAATTGTTTTCATATATTTGAAGAATTTTTACAAGGTAGATATCCCAAACCAGAAATGTTATAT |
| | AAACTAATTCTGGGTAACTTCACACAAAGTTGAAATAACATGGATATAGCACAGTTTAAAGGGTGATCTTTCACATTAGTCAAGA |
| | ATTTTTATCAGACAGATTCATGATTTAT[A/G]GCTTTTATAATAAAAATTATTTATTAAATGGGGTTGTTTATAGTTTGGACTAA |
| | TTGAGTTCATAGATCAAGGGAAAAAGCAGAAAAAGAGGTTTTACACTGTGTTCTTAAAATCTTGAGTTTTTGTGTTCCTAAAGGA |
| | CACATTTTAGTGAAAAAAATGTTTAAAAATTTATCTGGAAATTAGAATGTTTAAGGCCTAAAT |
| rs12143647 | CTCGAAGCAGGGCATGAGGCCCTTTTTGTTCTTGCCTGGTAGCTCTCCACTGTAGAAACTTTCCCTCCATAGCCCTGGATGCTCC |
| | AGCCACACAGTGCTAGCTGCCTTTCCCCAGCTCCCTTGTACTCACGTGCTTCTGCGTTTTTCCTCTGCCTGCCCTCCCCTTCCTG |
| | TGCCCTTTGTTTTCCTGTAAACTGCCTCT[C/T]CACATGAAGCTCCAGCTCAGATACCACCTCCTTCAAGAGACCATCCTTGACC |
| | GGGCGTAGTGGCTCACACCTGTGATCCCAGCACTTTGGGAGGCCAAGGCAGGTGAATCGCTTGAGGCCAGGAGTTCAAGACCAGC |
| | CTGGGCAACATGACAAAACCCCATCTTAAAAAAAAAAAAAGNCCATTTTTTCTCCTAATGTTC |
| rs7550277 | AACTTGTCATTTACAAACACCTGTAAATNCGAACTCTACTTTTTAATATGGCATTACAACTTACAAGATTTGTATGTATGTATGC |
| | AATTAACCATGATACAATGGCAGCATAAAATGGCACCGGTAAAGTGTCAGGCACACAGCCTATTGCAGAGTGAACACTCACATTT |
| | TGTTTCCACGTGCCCCACCTCTTTTCTTGA[C/T]TTAAAGGATTTCTACTGGGCAAATGATAAGAGAAACAAGACACTTTTCTGT |
| | CCTTAAGGAACTTAGACTGGTAAAGAGGATAAAATAAACACAGACAAAGCCAGGCAGGATATACATTTTGAGAGCAGAATGAATC |
| | CAGGTGATATAAGAACACAAAGGAGGAAGACCATGTGCCAAGTTCCCTGAGGACATCAGGATCA |

### FURTHER REFERENCES

1. Kessler R C, Berglund P, Demler O, Jin R, Merikangas K R, Walters E E. Lifetime prevalence and age-of-onset distributions of DSM-IV disorders in the National Comorbidity Survey Replication. Arch Gen Psychiatry. June 2005; 62(6):593-602.
2. Nierenberg A A, Gray S M, Grandin L D. Mood disorders and suicide. J Clin Psychiatry. 2001; 62 Suppl 25:27-30.
3. Teicher M H, Glod C, Cole J O. Emergence of intense suicidal preoccupation during fluoxetine treatment. Am J Psychiatry. February 1990; 147(2):207-210.
4. Masand P, Gupta S, Dewan M. Suicidal ideation related to fluoxetine treatment. N Engl J Med. Feb. 7 1991; 324(6):420.
5. Wirshing W C, Van Putten T, Rosenberg J, Marder S, Ames D, Hicks-Gray T. Fluoxetine, akathisia, and suicidality: is there a causal connection? Arch Gen Psychiatry. July 1992; 49(7):580-581.
6. Licinio J, Wong M L. Depression, antidepressants and suicidality: a critical appraisal. Nat Rev Drug Discov. February 2005; 4(2):165-171.
7. Hall W D, Mant A, Mitchell P B, Rendle V A, Hickie I B, McManus P. Association between antidepressant prescribing and suicide in Australia, 1991-2000: trend analysis. Bmj. May 10, 2003; 326(7397):1008.
8. Morgan O W, Griffiths C, Majeed A. Association between mortality from suicide in England and antidepressant prescribing: an ecological study. BMC Public Health. Dec. 21 2004; 4:63.
9. Nakagawa A, Grunebaum M F, Ellis S P, et al. Association of suicide and antidepressant prescription rates in Japan, 1999-2003. J Clin Psychiatry. June 2007; 68(6):908-916.
10. Angst J, Angst F, Gerber-Werder R, Gamma A. Suicide in 406 mood-disorder patients with and without long-term medication: a 40 to 44 years' follow-up. Arch Suicide Res. 2005; 9(3):279-300.
11. Perlis R H, Beasley C M, Jr., Wines J D, Jr., et al. Treatment-associated suicidal ideation and adverse effects in an open, multicenter trial of fluoxetine for major depressive episodes. Psychother Psychosom. 2007; 76(1):40-46.
12. Jick H, Kaye J A, Jick S S. Antidepressants and the risk of suicidal behaviors. Jama. Jul. 21 2004; 292(3):338-343.
13. Hammad T A, Laughren T, Racoosin J. Suicidality in Pediatric Patients Treated With Antidepressant Drugs 10.1001/archpsyc.63.3.332. Arch Gen Psychiatry. Mar. 1, 2006 2006; 63(3):332-339.
14. Vitiello B, Swedo S. Antidepressant Medications in Children 10.1056/NEJMp038248. N Engl J Med. Apr. 8, 2004 2004; 350(15):1489-1491.
15. Marshall E. ANTIDEPRESSANTS AND CHILDREN: Buried Data Can Be Hazardous to a Company's Health 10.1126/science.304.5677.1576. Science. Jun. 11, 2004 2004; 304(5677):1576-1577.
16. Libby A M, Brent D A, Morrato E H, Orton H D, Allen R, Valuck R J. Decline in Treatment of Pediatric Depression After FDA Advisory on Risk of Suicidality With SSRIs 10.1176/appi.ajp.164.6.884. Am J Psychiatry. Jun. 1, 2007 2007; 164(6):884-891.
17. Valuck R J, Libby A M, Orton H D, Morrato E H, Allen R, Baldessarini R J. Spillover Effects on Treatment of Adult Depression in Primary Care After FDA Advisory on Risk of Pediatric Suicidality With SSRIs. Am J Psychiatry. Aug. 1, 2007 2007; 164(8):1198-1205.
18. Gibbons R D, Brown C H, Hur K, et al. Early evidence on the effects of regulators' suicidality warnings on SSRI prescriptions and suicide in children and adolescents. Am J Psychiatry. September 2007; 164(9):1356-1363.
19. Brent D A, Mann J J. Family genetic studies, suicide, and suicidal behavior. Am J Med Genet C Semin Med Genet. Feb. 15 2005; 133(1):13-24.
20. Statham D J, Heath A C, Madden P A, et al. Suicidal behaviour: an epidemiological and genetic study. Psychol Med. July 1998; 28(4):839-855.
21. Sherry S T, Ward M H, Kholodov M, et al. dbSNP: the NCBI database of genetic variation. Nucleic Acids Res. Jan. 1 2001; 29(1):308-311.
22. Sambrook J, Fritsch E F, Maniatis T. Molecular Cloning: A Laboratory Manual. New
23. Kim S, Misra A. SNP genotyping: technologies and biomedical applications. Annu Rev Biomed Eng. 2007; 9:289-320.
24. Wang L, Luhm R, Lei M. SNP and mutation analysis. Adv Exp Med Biol. 2007; 593:105-116.
25. Hill W G, Robertson A. Linkage disequilibrium in finite populations. Theoretical and Applied Genetics. 1968; 38:226-231.
26. Hamilton M. A rating scale for depression. J Neurol Neurosurg Psychiatry. February 1960; 23:56-62.
27. Hamilton M. Development of a rating scale for primary depressive illness. Br J Soc Clin Psychol. December 1967; 6(4):278-296.
28. Trivedi M H, Rush A J, Ibrahim H M, et al. The Inventory of Depressive Symptomatology, Clinician Rating (IDS-C) and Self-Report (IDS-SR), and the Quick Inventory of Depressive Symptomatology, Clinician Rating (QIDS-C) and Self-Report (QIDS-SR) in public sector patients with mood disorders: a psychometric evaluation. Psychol Med. January 2004; 34(1):73-82.
29. Rush A J, Trivedi M H, Ibrahim H M, et al. The 16-Item Quick Inventory of Depressive Symptomatology (QIDS), clinician rating (QIDS-C), and self-report (QIDS-SR): a psychometric evaluation in patients with chronic major depression. Biol Psychiatry. Sep. 1 2003; 54(5):573-583.
30. Rush A J, Trivedi M H, Carmody T J, et al. Self-reported depressive symptom measures: sensitivity to detecting change in a randomized, controlled trial of chronically depressed, nonpsychotic outpatients. Neuropsychopharmacology. February 2005; 30(2):405-416.
31. Rush A J, Bernstein I H, Trivedi M H, et al. An evaluation of the quick inventory of depressive symptomatology and the hamilton rating scale for depression: a sequenced treatment alternatives to relieve depression trial report. Biol Psychiatry. Mar. 15 2006; 59(6):493-501.
32. Laje G, Paddock S, Manji H, et al. Genetic Markers of Suicidal Ideation Emerging During Citalopram Treatment of Major Depression. Am J. Psychiatry. Oct. 1, 2007 2007; 164(10)1530-1538.
33. Wigginton J E, Cutler D J, Abecasis G R. A note on exact tests of Hardy-Weinberg equilibrium. Am J Hum Genet. May 2005; 76(5):887-893.

1. A method of diagnosing a predisposition for or the occurrence of treatment emergent suicidal ideation in an individual, the method comprising determining the presence or absence of one or more of the SNPs selected from the group consisting of SEQ ID NOs: 8, 18, 19, 47, 50, 52, 56, 64, 73, 86, 87, 92, 94, 95, 97 and 99.

2. The method of claim 1, wherein the SNP comprises SEQ ID NO: 8.

3. The method of claim 1, wherein exactly 2, 3, 4, 5, 10 or 15 SNPs are used.

4. The method of claim 1, wherein, in addition to one or more of the SNPs selected from the group consisting of SEQ ID NOs: 8, 18, 19, 47, 50, 52, 56, 64, 73, 86, 87, 92, 94, 95, 97 and 99, one or more of the SNPs selected from the group consisting of SEQ ID NOs: 1 to 7, 9 to 17, 20 to 46, 48, 49, 51, 53 to 55, 57 to 63, 65 to 72, 74 to 85, 88 to 91, 93, 96, 98 and 100 are used.

5. The method of claim 1, wherein instead of or in addition to a SNP as defined in claim 1, a corresponding tagging SNP is used, wherein said tagging SNP is provided in Table 4.

6. A method of diagnosing a predisposition for or the occurrence of treatment emergent suicidal ideation in an individual, the method comprising determining the presence or absence of one or more SNPs selected from the SNPs selected from the group consisting of SEQ ID NOs: 1 to 100 in a sample obtained from a patient, wherein presence of the respective SNP(s) is/are indicative of said predisposition for or said occurrence of treatment emergent suicidal ideation.

7. The method of claim 6, wherein the number of SNPs used is selected from the group consisting of 2, 3, 4, 5, 10, 15, 16 or 18 SNPs.

8. The method of claim 6, wherein said one or more SNPs comprise the SNP defined by SEQ ID NO: 1.

9. The method of claim 6, wherein said SNPs comprise the SNPs selected from the group consisting of:
(a) SEQ ID NOs: 1, 3, 66 and 78; or
(b) SEQ ID NOs: 1, 3, 6, 9, 21, 23, 29, 32, 38, 53, 55, 59, 61, 62, 66, 76, 78 and 94.

10. The method of claim 1, wherein said determining is effected by allele specific hybridization, allele specific oligonucleotide ligation, primer extension, minisequencing, mass spectroscopy, heteroduplex analysis, single strand conformational polymorphism, denaturing gradient gel electrophoresis, oligonucleotide microarray analysis, temperature gradient gel electrophoresis or combinations thereof.

11. The method of claim 1, wherein said treatment emergent suicidal ideation occurs or may occur in a patient being administered selective serotonin re-uptake inhibitors, selective noradrenalin re-uptake inhibitors, dual serotonin and noradrenalin re-uptake inhibitors and/or tricyclic antidepressants, and optionally being administered neuroleptics, mood stabilizers and/or benzodiazepines.

12. The method of claim 1, wherein said individual is a child or adolescent.

13. The method of claim 1, wherein said determining comprises isolating a nucleic acid from said sample.

14. A kit comprising
(a) one or more agents suitable for determining presence or absence of two or more SNPs selected from the group consisting of SEQ ID NOs: 1 to 100; and
(b) optionally a manual with instructions for performing the method of claim 1.

15. The kit of claim 14, wherein said agents are selected from primers and probes for determining presence or absence of one or more SNPs selected from the group consisting of SEQ ID NOs: 1 to 100.

16. The kit of claim 14, wherein said two or more SNPs are selected from the group consisting of 2, 3, 4, 5, 10, 15, 16, 18, 20, 30, 40, 50, 60, 70, 80, 90 or 100 SNPs.

17. A kit comprising
(a) one or more agents suitable for determining presence or absence of two or more SNPs selected from the group consisting of SEQ ID NOs: 1 to 100; and
(b) optionally a manual with instructions for performing the method claim 1, wherein said SNPs are the SNPs defined in claim 1.

18. (canceled)

19. A method of diagnosing a predisposition for or the occurrence of treatment emergent suicidal ideation in an individual, the method comprising determining the presence or absence of one or more SNPs selected from:
(a) one or more SNPs selected from the group consisting of SEQ ID NOs: 1 to 100;
(b) at least one tagging SNP as set forth in Table 4; or
(c) one or more SNPs selected from the group consisting of SEQ ID NOs: 1 to 100 and at least one tagging SNP as set forth in Table 4; and
in a sample obtained from a patient, wherein presence of the respective SNP(s) is/are indicative of said predisposition for or said occurrence of treatment emergent suicidal ideation.

20. A kit comprising
(a) one or more agents suitable for determining presence or absence of:
(i) two or more SNPs selected from the group consisting of SEQ ID NOs: 1 to 100;
(ii) at least one tagging SNP as set forth in Table 4; or
(iii) two or more SNPs selected from the group consisting of SEQ ID NOs: 1 to 100 and at least one tagging SNP as set forth in Table 4; and
(b) optionally a manual with instructions for performing the method claim 1.
